(19) United States
(12) Patent Application Publication
(10) Pub. No.: US 2018/0148784 A1
(43) Pub. Date: May 31, 2018

(54) METHOD FOR IN VITRO DIAGNOSIS OF DEMENTIA WITH LEWY BODIES USING ALPHASYNUCLEIN GENE TRANSCRIPTS

(71) Applicants: FUNDACIÓ INSTITUT D'INVESTIGACIÓ EN CIÈNCIES DE LA SALUT GERMANS TRIAS I PUJOL, Badalona (ES); UNIVERSITAT AUTÒNOMA DE BARCELONA, Bellaterra (ES)

(72) Inventors: Katrin BEYER, Cervelló (ES); Aurelio ARIZA FERNÁNDEZ, Barcelona (ES)

(21) Appl. No.: 15/572,345

(22) PCT Filed: May 6, 2016
(86) PCT No.: PCT/EP2016/060177

§ 371 Date: Nov. 7, 2017

(30) Foreign Application Priority Data
May 8, 2015 (EP) 15382241.6

(51) Int. Cl. C12Q 1/6883

(52) U.S. Cl. CPC C12Q 1/6883; C12Q 2600/106; C12Q 2600/158

(57) ABSTRACT

The present invention provides a method for the in vitro diagnosis of dementia with Lewy bodies in a human patient comprising the step of determining the amount of transcripts SNCAtv3 (SEQ ID NO: 3) and SNCAtv2 (SEQ ID NO: 2) of the human alpha-synuclein gene (SNCA) in a biological sample obtained from the patient, wherein when the amount of both transcripts determined for the patient is reduced with respect to a reference value, this is indicative of the presence of dementia with Lewy bodies in the patient. The invention further provides means to determine the amount of said transcripts, as well as a method to stablish the response of a patient which has been diagnosed with dementia with Lewy bodies to a medical regime for its treatment by determining the amount of transcripts SNCAtv3 and SNCAtv2 in a biological sample from the patient before and after the treatment.

### BACKGROUND ART

The present invention relates to the field of medicine, particularly to neurodegenerative disorders, specifically to a method of in vitro diagnosis of synucleinopathies using transcripts of the alpha-synuclein gene (SNCA).

Synucleinopathies are diseases characterized by the presence of neuronal proteinaceous inclusions called Lewy bodies (LB). Lewy bodies and Lewy neurites are formed fundamentally by the protein alpha-synuclein. Several synucleinopathies are known, but by far the more relevant disorders included in this group are Parkinson's disease (PD) and Dementia with Lewy bodies (DLB). While PD is most common progressive movement disorder at old age, DLB is the second most frequent cause of dementia after Alzheimer's disease. When it was first described, it was believed that the DLB was a rare disorder; however, in recent years extensive research on this disease has revealed that it is present in 10-15% of autopsied cases. The main symptoms of DLB include fluctuating cognitive impairment, recurrent visual hallucinations and Parkinsonism.

The diagnosis of the various forms of synucleinopathies still relies on an exact medical and family history, an accurate examination of the patient, and on the skills of the examiner. Neuroimaging (DaTscan) has been introduced, but these imaging techniques often require use of radioactive compounds and are not cost-effective, so that their application is limited. Biological diagnosis markers would be of help, but so far none have been found to have clinical significance.

Further, because of clinical overlap, differential diagnosis of PD and DLB is very difficult. Parkinsonism is the predominant symptom of PD, but it can be indistinguishable from the parkinsonism of DLB. DLB could be the same disease as PD but with widespread cortical pathological states, leading to dementia, fluctuating cognition, and the characteristic visual hallucinations. Additionally, many symptoms of Alzheimer's disease (AD) also overlap with DLB, which entails frequent misdiagnosis of DLB. Treatment of PD relies on administration of agents that increase the levels of dopamine (Levodopa). However, patients suffering of DLB benefit from administration of alternative/additional drugs, in particular, acetylcholine esterase inhibitors. It follows that there exists a need to provide cost-effective methods for the differential diagnosis of PD and DLB in order to be able to apply the most effective treatment for each patient.

Patent EP2539461 discloses that specific alterations in Butyrylcholinesterase (BChE) gene are specifically related to DLB. This discovery allows for the differential diagnosis of patients suffering from DLB by determining the genotype of particular alterations in BChE gene in a blood sample. While being a substantial improvement for this field, the disclosed diagnosis may only identify a small proportion of DLB patients. Indeed, recent results have shown that this method identifies only around 10% of DLB patients, so that there is still a substantial proportion of DLB patients that are not diagnosed.

On the other side, several studies have attempted to use the alpha-synuclein protein as a biomarker in human biological fluids for detection of DLB. For example, in WO2011104696 are disclosed monoclonal or polyclonal antibodies that detect protofibrilar or oligomeric soluble forms of alpha-synuclein in biological fluids, e.g. blood, and the use of these antibodies for diagnosing neurodegenerative synucleinopathies. Furthermore, WO201069603 discloses the use of antibodies obtained or isolated from a “pool” of elderly people without PD, which are sequenced and cloned in different vectors. Such antibodies have affinity for monomeric, oligomeric, aggregate forms, fragments and/or posttranslational modified forms of alpha-synuclein and allow detection of DLB.

Finally, WO9950300 discloses methods for differentiation of synucleinopathies from other neurodegenerative diseases by detecting filamentous aggregates of synucleins. In this document, it is mentioned speculatively that the diagnosis could be accomplished by detecting levels of synuclein nucleic acids by performing known techniques, such as polymerase chain reaction (PCR), ligase chain reaction (LCR), isothermal nucleic acid amplification (NASBA) or polymerase chain reaction with reverse transcription (RT-PCR). The above diagnoses are either not reliable or they need of the proliferation of aggregate forms of SNCA (which are found in advanced stages of the synucleinopathies), or both. An early diagnosis is more desirable in terms of management of the disorder. Furthermore, reliable differential diagnosis of DLB patients (as distinguished from PD patients) would allow recommending the most effective treatment regime for these patients.

Therefore, there is still a need to provide means for an early and accurate identification of patients suffering from a synucleinopathy and also for the differential diagnosis of the different synucleinopathies to be used in the common clinical practice.

### SUMMARY OF THE INVENTION

The present invention relates to the use of transcripts of the alpha-synuclein gene as biomarkers in human biological fluids for in vitro diagnosis of synucleinopathies.

It is known that the gene encoding the alpha-synuclein protein, called SNCA (SEQ ID NO: 1), presents a complex splicing processing. After conducting extensive studies, the present inventors have surprisingly found that two of said transcripts (but not all) provide information for the in vitro diagnosis of synucleinopathies. The present invention is based on using these two transcripts of the SNCA gene, called SNCAtv2 (SEQ ID NO: 2) and SNCAtv3 (SEQ ID NO: 3), as biomarkers in biological fluids for the diagnosis of synucleinopathies. These transcripts SNCAtv2 and SNCAtv3 differ from each other and from the major transcript SNCAtv1 by differential incorporation of one of the four exons located in the 5′ untranslated part of the SNCA gene.

Therefore, in a first aspect, the present invention provides a method for the in vitro diagnosis of a synucleinopathy in a human patient comprising the step of determining the amount of at least one transcript of the human alpha-synuclein gene (SNCA) selected from the group consisting of SNCAtv2 (SEQ ID NO: 2) and SNCAtv3 (SEQ ID NO: 3) in a biological sample obtained from the patient.

By using this method patients that suffer from a synucleinopathy are identified. Diagnosis is achieved in patients that have recently developed the disease, thus enabling an early diagnosis of the synucleinopathy. This is a big advantage with respect to known methods. Early diagnosis increases the therapeutic margin to reduce or stop the disease progression, thus improving live quality and prognosis for the patient. Further, the present method allows for differential diagnosis of DLB versus Parkinson disease at a very early stage.

In a second aspect, the present invention provides for the use of means for determining the amount of at least one transcript of the human SNCA selected from the group consisting of SNCAtv2 and SNCAtv3 in a biological sample for the diagnosis of a synucleinopathy in the method as defined in the first aspect of the invention.

In a further aspect, the present invention provides for the use of at least one human alpha-synuclein gene transcript selected from SNCAtv2 and SNCAtv3 for the diagnosis of synucleinopathies.

While providing for a reliable and early diagnosis of a synucleinopathy, the present diagnosis method is useful to a clinician, who is able to take appropriate decisions to treat the patient, i.e. to apply a medical regime that is appropriate for treating synucleinopathies. Thus, in another aspect, the invention is directed to a method of deciding or recommending to initiate a medical regime for the treatment of a synucleinopathy in a human patient, which method comprises diagnosing a synucleinopathy or determining whether the patient is suspicious of suffering a synucleinopathy by the method as defined in the first aspect of the invention, wherein (a) if the patient is diagnosed of suffering from a synucleinopathy, or of being suspicious of suffering from a synucleinopathy, then the initiation of the medical regimen is recommended, and (b) if the subject is diagnosed of not suffering from a synucleinopathy, the follow-up is performed optionally in consideration of the result of an examination of the patient by a physician.

The inventors have further found that the amount of SNCAtv2 and SNCAtv3 transcripts may serve to monitor synucleinopathies progression because said amount is inversely correlated with the alpha-synuclein aggregation rate in the brain of the patients (see example 4). As a result, the amount of SNCAtv2 and SNCAtv3 transcripts may also be useful for determining the response to a medical regime that is administered to the patient to treat a synucleinopathy.

Thus another aspect of the invention refers to a method to stablish the response of a human patient which has been diagnosed with a synucleinopathy to a medical regime for the treatment of the synucleinopathy, which method comprises determining the amount of at least one human alpha-synuclein gene transcript selected from the group consisting of SNCAtv3 and SNCAtv2 in a biological sample obtained from the patient being treated and comparing said amount of transcript(s) with the amount of transcript(s) determined for the same patient before the treatment or at an earlier phase of the treatment, wherein a difference in the amount of transcript(s) with respect to before the treatment or earlier phase of the treatment is indicative of a the response to the medical regime.

For a better understanding, the present invention is described below with reference to the accompanying figures, which are presented by way of example, and in no way meant to be limiting of the present invention.

### BRIEF DESCRIPTION OF THE FIGURES

FIG. 1 shows the change of expression of transcripts SNCAtv1 (SEQ ID NO: 4), SNCAtv2, SNCAtv3, and SNCA112 (SEQ ID NO: 5) in PD and DLB patients regarding healthy controls.

FIG. 2 shows the change in expression of transcripts SNCAtv1 (vertical lines), SNCAtv2 (diagonal), SNCAtv3 (squares) and SNCA112 (horizontal lines) at different groups of patients with DLB: who developed the disease before age 65 between 65 to 74 years and after 75 years compared to healthy matched controls age.

FIG. 3 shows the change in expression of transcripts SNCAtv1 (vertical lines), SNCAtv2 (horizontal lines), SNCAtv3 (diagonal) and SNCA112 (squares) in different groups of patients diagnosed with DLB: 0 to 1 year, 2 years, 3 to 4 years or over 4 years from diagnosis of the disease, compared to healthy controls.

FIG. 4 shows SNCAtv3 expression levels in blood during disease progression, namely, for less than 1 year, for 2 years, for 3-4 years and for more than 4 years since the onset of disease. The Y axis represents SNCAtv3 expression change

### DETAILED DESCRIPTION OF THE INVENTION

The term “synucleinopathies” as used herein refers to a group of disorders characterised by the abnormal accumulation of aggregates of alpha-synuclein protein in neurons, nerve fibres or glial cells. This group includes Parkinson's disease and dementia with Lewy bodies. Other rare disorders, such as multiple system atrophy and various neuroaxonal dystrophies also have alpha-synuclein pathologies.

As used herein “alpha-synuclein gene”, “SNCA” and “SNCA gene” are indistinctively used to refer to the alpha-synuclein gene, while alpha-synuclein is used to refer to the corresponding protein.

As used herein, the term “transcript” or “transcripts” refers to complete transcripts or specific regions thereof. That refers to complete molecules of ribonucleic acid (RNA) or to specific regions thereof.

As used herein, the terms “transcript SNCAtv1”, “transcript SNCAtv2”, “transcript SNCAtv3”, “transcript SNCA112”, “SNCAtv1”, “SNCAtv2”, “SNCAtv3” and“SNCA112” refer to regions or fragments present in the different RNAs derived or produced from the SNCA gene or to complete transcripts or RNA molecules derived or produced from the SNCA gene.

Herein, the terms “biological sample”, “biological fluid sample”, “biological fluid” and their plurals are used as equivalent and interchangeable for referring to liquids derived from an individual.

As used herein, the terms “DLB” and “Dementia with Lewy bodies” refer to any stage of DLB, regardless of the age of patient or time since diagnosis.

As mentioned above, the present invention provides, in a first aspect, a method for the in vitro diagnosis of synucleinopathies in a human patient comprising the step of determining the amount of at least one transcript of the human alpha-synuclein gene (SNCA) selected from the group consisting of SNCAtv2 (SEQ ID NO: 2) and SNCAtv3 (SEQ ID NO: 3) in a biological sample obtained from the patient.

In a particular embodiment of the first aspect of the invention, when the amount of the transcript(s) determined for the patient is reduced with respect to a reference value, this is indicative of a synucleinopathy in the patient.

The term “reference value” referred to in the method of the first aspect is to be understood as a predefined amount of SNCAtv2 or SNCAtv3 transcripts, which are derived from the amounts of said marker in a sample or group of samples. The samples are taken from a subject or group of control subjects that do not suffer from any neurological symptomatology. The skilled person in the art, making use of the general knowledge, is able to choose the subject or group of subjects more adequate for obtaining the reference value.

In one embodiment, the reference value is determined from a population of control subjects that do not show any synucleinopathy. According to the findings of the present inventors, when the amount of SNCAtv2 and SNCAtv3 in the patient being tested was significantly decreased when compared to the reference value obtained from subjects without neurological symptomatology, it would be indicative that the patient suffers from a synucleinopathy. From said data, the clinician is able to take appropriate decisions to treat the patient, i.e. to apply a therapy that is appropriate for synucleinopathies.

In a particular embodiment, the method of the invention comprises determining the amount of at least SNCAtv3 in the sample. In another embodiment, the method of the invention comprises determining both SNCAtv2 and SNCAtv3 transcripts. According to the findings of the present inventors, when the amount of both SNCAtv2 and SNCAtv3 in the subject being tested was significantly decreased compared to the reference value obtained from subjects without neurological symptomatology, it would be indicative that the patient suffers from DLB. Thus Since the method of the invention allows for differential diagnosis of DLB with respect to other synucleinopathies, in particular, with respect to Parkinson disease, by applying the present method the clinician may decide to recommend specific medical regimes that are indicated for treating DLB. In particular, the clinician may recommend administrating acetylcholine esterase inhibitors to the patient.

With the diagnostic method of synucleinopathies of the present invention the disease can be diagnosed at any stage and regardless of patient age. However, it is an advantage of the present invention that an early diagnosis of synucleinopathies is achieved. In particular embodiments, early diagnosis of DLB is achieved by use of the present method when determining the amount of SNCAtv2 and SNCAtv3. In one embodiment the diagnosis is performed in patients that have recently developed the disease, in particular the diagnosis is performed less than 3 years from the onset of the disease, more particularly less than 2 or less than 1 year from onset of disease. In another embodiment, the diagnosis is performed in patients of age below 75, or in patients of age below 65 years.

In a preferred embodiment, the sample of human biological fluid is selected from a group comprising blood, plasma, saliva, urine, cerebrospinal fluid, semen and derivatives thereof. The method of diagnosis of synucleinopathies of the present invention has as additional advantages that it is a non-invasive method, even in its early stages; also, it is a reliable and inexpensive method compared to current techniques.

Determining the amount of SNCAtv2 and/or SNCAtv3 transcripts can be performed by any method known to the skilled person, provided that said method permits the detection and quantification of RNA in a biological sample. Included among the examples of these procedures are PCR, quantitative real-time PCR (QPCR), multiplex PCR, NASBA, LCR, RT-PCR, RNA sequencing, array hybridization or “Northern” transfer, or combinations of these. In a preferred embodiment, the determination of the amount of the SNCA transcripts is performed by quantitative real-time PCR.

In most methods of detection and quantification of RNA mentioned above, before performing this procedure it is necessary to convert the RNA to complementary DNA (cDNA). This conversion is accomplished by known techniques by skilled in the art, such as reverse transcription, among others.

Furthermore, in most procedures that can be used in the method of the present invention, the use of primers is required to detect and/or amplify transcripts of interest. A skilled artisan would get easily and directly the sequence of the primers that can be used from the sequence of transcripts to be analysed. In a preferred embodiment of the present invention, the primer sequences are derived from the sequence of the transcript SNCAtv2 (SEQ ID NO: 2). In another preferred embodiment, the primer sequences are obtained from SNCAtv3 transcript sequence (SEQ ID NO: 3). In a further embodiment the primers used for determining the amount of SNCAtv2 are those with SEQ ID NO: 6 and SEQ ID NO: 8, while the primers used for determining the amount of SNCAtv3 are those with SEQ ID NO: 7 and SEQ ID NO: 8.

The present invention requires comparing the amount of the SNCAtv2 and/or SNCAtv3 transcript in a sample obtained from the patient with a reference value. If the amount of the transcript(s) determined in patient's sample is significantly reduced with respect to the reference value, this is indicative of the presence of a synucleinopathy in the patient. As mentioned above, the reference value is determined from a population of control subjects that do not show neurological symptomatology, in particular subjects that do not suffer from any synucleinopathy. The skilled person may use any available method to establish said comparison. For instance, when qPCR is used for determining the amount of the transcripts, the comparison between the amount of transcripts in the patient's and the amount of transcripts in the control subjects may be established by using the comparative Ct method. The “Ct” or “Ct value” of a qPCT reaction for a given target nucleic acid has the sense generally given in the art, i.e., the cycle threshold value. The Ct value means the number of PCR cycles where the reporter dye signal is sufficiently high to cross an automatically or manually determined threshold value, and it is a relative measure of the concentration of nucleic acid target in the qPCR reaction.

The comparative Ct method is also known as deltadeltaCt (2^{-ΔΔCT}) method, where:

ΔΔCt=ΔCt(patient)−ΔCt(control subjects), and
ΔCt=Ct(transcript of interest)−Ct(transcript of the housekeeping gene)

The housekeeping gene may be selected according to parameters well known to the skilled person. In particular embodiments, the housekeeping gene is beta-actin and hydroxymethylbilan. In a further particular embodiment, DLB is diagnosed when the reduction of the amount of the transcript(s) determined for the patient with respect to their respective reference values, wherein the reference value is the mean amount of transcript in control subjects, is calculated by the deltadeltaCt method, and said deltadeltaCt method yields a coefficient below 0.5. In one embodiment the tested subject is diagnosed with a synucleinopathy when the comparative Ct method yields a coefficient below 0.5 for SNCAtv3. In a particular embodiment, the patient is diagnosed with DLB when the comparative Ct method yields a coefficient below 0.5 for both SNCAtv2 and SNCAtv3 transcripts.

As mentioned above, a second aspect of the invention provides for use of means for determining the amount of at least one transcript of the human alpha-synuclein gene selected from the group consisting of SNCAtv2 and SNCAtv3 in a biological sample for the diagnosis of synucleinopathies in the method as defined in the first aspect. A particular embodiment refers to use of means for determining the amount of both SNCAtv2 and SNCAtv3 transcripts in a biological sample for the diagnosis of DLB. In certain embodiments said means are primers to determine the amount of the transcripts by qPCR. In particular embodiments, the primers used for determining the amount of SNCAtv2 are those with SEQ ID NO: 6 and SEQ ID NO: 8, while the primers used for determining the amount of SNCAtv3 are those with, SEQ ID NO: 7 and SEQ ID NO: 8. Thus, in a particular embodiment the means comprise at least one pair of primers selected from SEQ ID NO: 6/SEQ ID NO: 8 and SEQ ID NO: 7/SEQ ID NO: 8. In other embodiments the means include both pairs of primers defined above. In further particular embodiments any of the above means are comprised in a kit for the diagnosis of synucleinopathies, particularly for the differential diagnosis of DLB.

As will be evident for the skilled person, the above means, included or not in a kit, may also be employed for determining the amount of SNCAtv2 and/or SNCAtv3 transcripts for determining the progression of the synucleinopathy, deciding or recommending initiating a medical regime for the treatment of the synucleinopathy in the tested subject or stablishing the response of a patient to a medical regime for the treatment of the synucleinopathy. In particular embodiments, the above means are for determining the amount of SNCAtv2 and SNCAtv3 transcripts for determining the progression of the DLB, deciding or recommending initiating a medical regime for the treatment of DLB in the tested subject or stablishing the response of a patient to a medical regime for the treatment of DLB.

As mentioned above, the present invention provides the use of at least one human alpha-synuclein gene transcript selected from SNCAtv2 and SNCAtv3 for the diagnosis of synucleinopathies. In a particular embodiment the invention provides use SNCAtv3 for the diagnosis of synucleinopathies. In another particular embodiment the invention provides use of both SNCAtv2 and SNCAtv3 alpha-synuclein gene transcripts for the differential diagnosis of DLB.

The present invention also contemplates using SNCAtv2 and SNCAtv3 gene transcripts for the differential diagnosis of DLB in combination with at least one other marker known as being indicative of DLB. This may be rephrased as a method for the in vitro diagnosis of a DLB in a human patient comprising the step of determining the amount of SNCAtv3 and SNCAtv2 in a biological sample obtained from the patient in combination with determining at least one other marker known as being indicative of DLB. In particular embodiments, the other marker is one of the polymorphic sites in BChE gene disclosed in EP2539461. In another particular embodiment, the other marker is the polymorphic site at position 68974 in BChE gene as defined by NCBI Accession Number NG_009031 (i.e. position 934 in SEQ ID NO: 9, which corresponds to SEQ ID NO: 28 of EP2539461). In other particular embodiments, in addition to the polymorphic site at position 68974 in BChE gene, other variations in BChE gene are detected selected from the group consisting of the polymorphic sites at position 3687, 4206, 4443 and the poly-thymine region at positions 4780 to 4786 in NCBI Accession Number NG_009031 (i.e. positions 3687, 4206, 4443 and 4780-4786 respectively in SEQ ID NO: 10, which corresponds to SEQ ID NO: 1 of EP2539461).

The invention also provides a method of deciding or recommending to initiate a medical regime for the treatment of a synucleinopathy in a patient by determining the amount of at least one of SNCAtv2 and SNCAtv3 transcripts. In a particular embodiment said method comprises the steps of (a) determining the amount of at least one human alpha-synuclein gene transcript selected from SNCAtv2 and SNCAtv3 in a biological sample obtained from the patient; and (b) comparing the level obtained in step (a) with a reference value, wherein if the amount of transcript(s) detected in step (a) is lower than the reference value it is indicative that the subject would benefit from a medical regimen for the treatment of synucleinopathies. In particular embodiments, the amount of SNCAtv3 is determined for deciding on the medical regime for the treatment of synucleinopathies. In further embodiments, the amount of both SNCAtv2 and SNCAtv3 is determined and, when both SNCAtv2 and SNCAtv3 are lower than a reference value, it is indicative that the subject would benefit from a medical regimen for the treatment of DLB.

Lastly, the invention is also directed to the use of at least one of SNCAtv2 or SNCAtv3 transcripts to determine synucleinopathies progression in a patient, and also to a method of stablishing the response of a patient to a medical regime for the treatment of synucleinopathies by determining the amount of at least one of SNCAtv2 or SNCAtv3 transcripts. In a particular embodiment the method to stablish the response of a patient which has been diagnosed with a synucleinopathy to a medical regime for the treatment of the synucleinopathy comprises determining the amount of at least one human alpha-synuclein gene transcript selected from the group consisting of SNCAtv3 and SNCAtv2 in a biological sample obtained from the patient being treated and comparing said amount of transcript(s) with the amount of transcript(s) determined for the same patient before the treatment or at an earlier phase of the treatment, wherein an increase of the amount of transcript(s) with respect to before the treatment or earlier phase of the treatment is indicative of a good response to the medical regime. In particular embodiments, the transcript for determining disease progression or stablishing response to a medical regime is SNCAtv3. In some embodiments, both SNCAtv2 and SNCAtv3 transcripts are determined for determining DLB progression or stablishing response to a medical regime for the treatment of DLB.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### Example 1. Study of the Expression of Transcripts SNCAtv1, SNCAtv2, SNCAtv3 and SNCA112 in Patients Diagnosed with PD, DLB and Control Subjects (Control Subjects Did not Present any Neurological Symptomatology)

Expression of four transcripts of the alpha-synuclein gene was analysed: SNCAtv1, SNCAtv2, SNCAtv3 and SNCA112, in control subjects and patients diagnosed with DLB or PD. For this purpose blood samples of 8 control subjects, 32 DLB patients and 12 PD patients were collected. The clinical diagnosis of the individuals was conducted in the Departments of Neurology of the Hospitals Germans Trias i Pujol and Bellvitge (Spain).

RNA from these blood samples was isolated by methods known in the technique and converted to cDNA (complementary DNA).

For this, 2.5 ml of peripheral blood were extracted from each of the individuals included in the study. This blood was collected in PAXgene Blood RNA tubes (Preanalytix, Hombrechtikon, Switzerland). RNA was extracted using the PAXgene Blood RNA Kit (Preanalytix, Hombrechtikon, Switzerland) following the manufacturer's instructions. Briefly, the tubes were centrifuged at 4500 g and the obtained pellet of sedimented blood cells was washed 2 to 3 times. After washing, the blood cells were lysed and the resulting solution was introduced into the columns included in the kit mentioned above and nucleic acids contained in the sample bound to the columns while the remaining contaminants were eluted. Then, the DNA was eliminated by application of DNAses to the columns. Prior to the recovery of RNA several cycles of washing and centrifugation were performed. Finally, RNA was eluted using 80 μL of elution buffer included in the above mentioned kit.

s RNA concentration of the different samples was measured by applying 1 to 2 μL in a DS-11 spectrophotometer (DeNovix, Willington, Del., United States).

Based on these concentrations of RNA, reverse transcription (conversion of RNA to complementary DNA) was carried out by the use of the kit “Ready-kit To Go™ You-Prime First-Strand Beads” (GE Healthcare Life Sciences, Uppsala, Sweden) and instructions provided by the manufacturer. For this, 1 mg of RNA was diluted in a final volume of 32 μL and afterwards denatured by applying a temperature 65° C. for 10 minutes. After denaturation, said RNA and 1 μL of random primers were added to the tubes provided by the manufacturer in the kit and these tubes were incubated for 1 hour at 37° C.

The cDNA obtained was analysed by the technique of real time polymerase chain reaction (QPCR) using the primers listed in Table 1 and the marker SYBR Green. Beta-actin and hydroxymethylbilane synthase genes were used as “housekeeping” genes to normalize the expression of transcripts of interest.

TABLE 1

| Primer Label | Primer sequence (5′ to 3′) | Comments |
|---|---|---|
| SNCA-1U (SEQ ID NO: 7) | ATCCAGGAACAGCTGTCTTC | Specific primer for |
| | | transcript SNCAtv3 |
| SNCA-2aU (SEQ ID NO: 9) | TTCAAGCCTTCTGCCTTTCC | Specific primer for |
| | | transcript SNCAtv1 |
| SNCA-2bU (SEQ ID NO: 6) | AGTCGGAGTTGTGGAGAAGC | Specific primer for |
| | A | transcript SNCAtv2 |
| SNCA-4L (SEQ ID NO: 8) | ACCACTGCTCCTCCAACAT | Generic primer for |
| | | the studied SNCA |
| | | transcripts |
| SNCA-3U (SEQ ID NO: 10) | GTGCATGGTGTGGCAACA | Generic primer for |
| | | the studied SNCA |
| | | transcripts |
| SNCA-4/6L (SEQ ID NO: 11) | ATACCCTTCCTTGCCCAAC | Specific primer |
| | | transcript SNCA112 |
| β-actin U1 (SEQ ID NO: 12) | CGAGAAGATGACCCAGATCA | β-actin primer |
| b-actin L1 (SEQ ID NO: 13) | TACATGGCTGGGGTGTTGAA | β-actin primer |
| PBGD_U1 (SEQ ID NO: 14) | ACACACAGCCTACTTTCCAAG | Primer for |
| | | hydroxymethylbilan |
| | | synthase |
| PBGD_L1 (SEQ ID NO: 15) | TCAATGTTGCCACCACACTGT | Primer for |
| | | hydroxymethylbilan |
| | | synthase |

The reaction was carried out using 0.1 mL tubes (Strips Tubes and Cups, Qiagen, Hilden, Germany) and the QuantiTect SYBR Green PCR kit (Qiagen, Hilden, Germany). The final reaction volume for each sample was 15 μL, including 16 pmol of each primer necessary for the reaction (see in Table 2 below primer combinations used) and 1 μL cDNA.

TABLE 2

| Combination of primers | Amplified transcript |
|---|---|
| SNCA-1U and SNCA-4L | SNCAtv3 |
| SNCA-2AU and SNCA-4L | SNCAtv1 |
| SNCA-2BU and SNCA-4L | SNCAtv2 |
| SNCA-3U and SNCA-4/6L | SNCA112 |
| β-actin U1 and β-actin L1 | β-actin |
| PBGD_U1 and PBGD_L1 | hydroxymethylbilan |
| | synthase |

Then the tubes were placed in the Rotor-Gene 6000 equipment (Qiagen, Hilden, Germany) for performing the QPCR using the rotor for 72 tubes and fixed the following protocol:
initial denaturation step at 95° C. for 15 minutes;
40 consecutive cycles of denaturation-annealing-amplification with the following structure of temperature variation (the fluorescence data were acquired in the last second of each of the cycles):
   95° C. for 15 seconds
   20 seconds: 54° C. for hydroxymethylbilan synthase, 56° C. for SNCA112 and β-actin, and 58° C. for SNCAtv1, SNCAtv2 and SNCAtv3.
   72° C. for 30 seconds

At the end of the protocol, a Ct value was obtained for each analysed transcript and for each sample. Each set of measurements performed on the Rotor-Gene 6000 equipment (Qiagen Hilden, Germany) was carried out in duplicate.

The data obtained were analysed using the methodology deltadeltaCt (ΔΔCT), based on that the PCR reaction takes place with the same efficiency for both transcripts those to be analysed and the “housekeeping” genes. To perform calculations corresponding to the ΔΔCt method the following Formulas 1, 2 and 3 were applied consecutively to the Ct values obtained previously:

Formula 1, applied between Ct values obtained for the different transcripts of interest, ie SNCAtv1, SNCAtv2, SNCAtv3 and SNCA112, and Ct values obtained for the transcripts of the “housekeeping” genes, beta-actin and hydroxymethylbilan synthase genes, within a sample or individual, either the group of patients with DLB or the group of healthy controls, permitting to obtain ΔCt for each transcript of interest in each individual:

ΔCt=Ct_{transcript of interest}−Ct_{transcript of the housekeeping gene}

Formula 2, applied between ΔCt values of the same transcript in patients with DLB and control subjects: ΔΔCt=ΔCtpatient−ΔCtcontrol

Formula 3: 2^{-ΔΔct}

After application of the formulas above, the value of variation of expression of each of the transcripts of interest was obtained in connection with the expression thereof in the control subjects.

According to the ΔΔCt methodology, it is considered that a decrease in expression is significant when the value obtained after normalization to the value of expression in control subjects is less than 0.5. Likewise, it is considered that an increase in expression is significant when the value obtained after normalization mentioned above, is greater than 1.5.

In this sense, in Table 3 are shown the average values of expression change of transcripts SNCAtv1, SNCAtv2, SNCAtv3 and SNCA112 regarding to their expression in control subjects with the confidence interval in parentheses (confidence level 95%). According to the aforesaid, the results shown in Table 3 were considered significant:
In the case of decreased expression compared to control subjects, the maximum value of the confidence interval was less than 0.5; and
In the case of increased expression relative to control subjects, the minimum value of the confidence interval was greater than 1.5.

TABLE 3

| | SNCAtv1 | SNCAtv2 | SNCAtv3 | SNCA112 |
|---|---|---|---|---|
| PD patients | 0.85 | 0.54 | 0.45 | 0.75 |
| (expression | (0.83-0.88) | (0.53-0.55) | (0.43-0.47) | (0.68-0.82) |
| variation of | | | | |
| the transcript) | | | | |
| DLB patients | 0.57 | 0.37 | 0.33 | 0.81 |
| (expression | (0.54-0.60) | (0.31-0.45) | (0.32-0.34) | (0.56-1.18) |
| variation of | | | | |
| the transcript) | | | | |

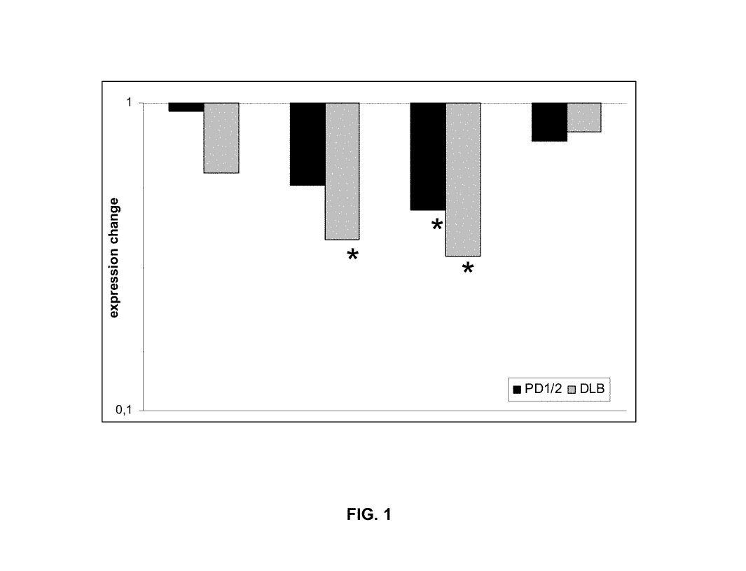

The results of Table 3 are represented schematically in FIG. 1.

Both Table 3 and FIG. 1 show that transcript SNCAtv3 showed a significant reduction of expression in patients with synucleinopathies (PD and DLB) with respect to control subjects. Additionally, it is shown that both SNCAtv2 and SNCAtv3 showed a significant reduction of expression in patients with DLB. However, it was noted that neither transcript SNCAtv1 and nor SNCA112 showed significant differences in expression between patients with PD or DLB and control subjects.

The sensitivity and specificity of SNCAtv2 and SNCAtv3 transcripts for the diagnosis of DLB were above 90%.

### Example 2. Influence of Age of Onset of the Disease in Patients with DLB on Expression of Transcripts SNCAtv1, SNCAtv2, SNCAtv3 and SNCA112

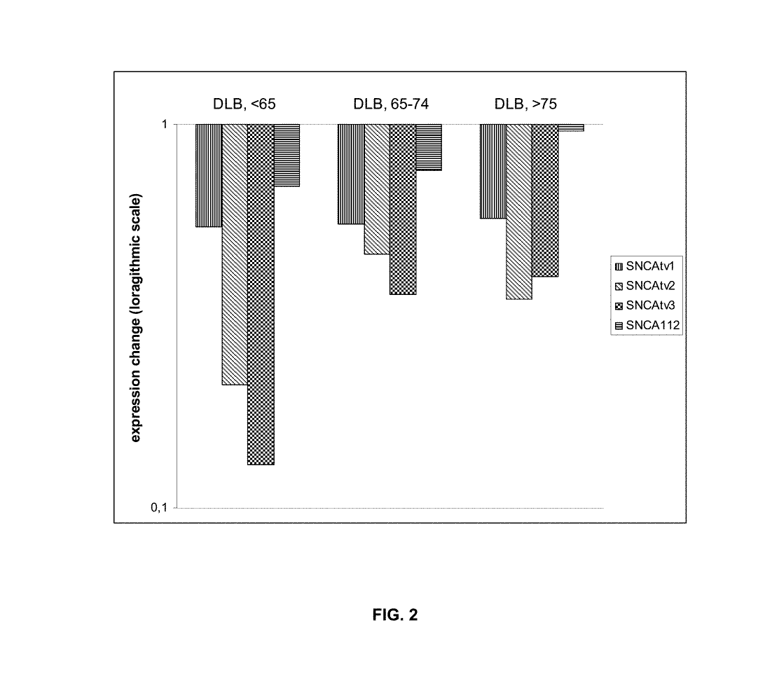

Data of variation of expression of transcripts SNCAtv1, SNCAtv2, SNCAtv3 and SNCA112 in patients with DLB normalized versus control subjects obtained in Example 1 were divided into three groups depending on the age of DLB beginning of the patients: before an age of 65 years (4 patients), between 65 and 74 years (16 patients) and after 75 years (12 patients). The results obtained are summarized in the following Table 4 and shown in FIG. 2.

TABLE 4

| | SNCAtv1 | SNCAtv2 | SNCAtv3 | SNCA112 |
|---|---|---|---|---|
| DLB patients | 0.54 | 0.21 | 0.13 | 0.69 |
| who debuted | (0.32-0.93) | (0.15-0.29) | (0.12-0.14) | (0.50-0.98) |
| with disease | | | | |
| before age of | | | | |
| 65 years | | | | |
| DLB patients | 0.55 | 0.46 | 0.36 | 0.76 |
| who debuted | (0.52-0.58) | (0.38-0.54) | (0.33-0.39) | (0.52-1.09) |
| with disease | | | | |
| between 65 | | | | |
| and 74 years | | | | |
| DLB patients | 0.57 | 0.35 | 0.4 | 0.96 |
| who debuted | (0.47-0.68) | (0.26-0.48) | (0.35-0.47) | (0.64-1.43) |
| with disease | | | | |
| after 75 years | | | | |

As in the case of Example 1, the data shown in Table 4 were significant:
In the case of decreased expression compared to control subjects, the maximum value of the confidence interval was less than 0.5; and
In the case of increased expression relative to control subjects, the minimum value of the confidence interval was greater than 1.5.

Both Table 4 and FIG. 2 show that transcripts SNCAtv2 and SNCAtv3 showed, in virtually all cases, a significant reduction of expression in DLB patients compared to control subjects. The only exception was observed in the group of patients with DLB debut aged between 65 and 74 years. In this group, the transcript SNCAtv2 showed no significant difference in its expression relative to control subjects, but a clear trend to reduced expression compared with control subjects. Therefore, expression of the transcripts SNCAtv2 and SNCAtv3 is reduced in patients with DLB independently on age of onset of the disease. Instead, it was observed that transcripts SNCAtv1 and SNCA112 showed no significant differences in expression between patients with DLB and control subjects in either group.

Both the sensitivity and specificity of SNCAtv2 and SNCAtv3 transcripts for the diagnosis of DLB were above 90% in all groups studied.

### Example 3. Influence of Time Since Diagnosis of DLB on the Expression of SNCAtv1, SNCAtv2, SNCAtv3 and SNCA112 Transcripts

Data of variation of expression of transcripts SNCAtv1, SNCAtv2, SNCAtv3 and SNCA112 in patients with DLB normalized versus control subjects obtained in Example 1 were divided into four groups according to time since diagnosis: 0 to 1 year (6 patients), 2 years (9 patients), among 3 and 4 years (7 patients) and more than 4 years (6 patients).

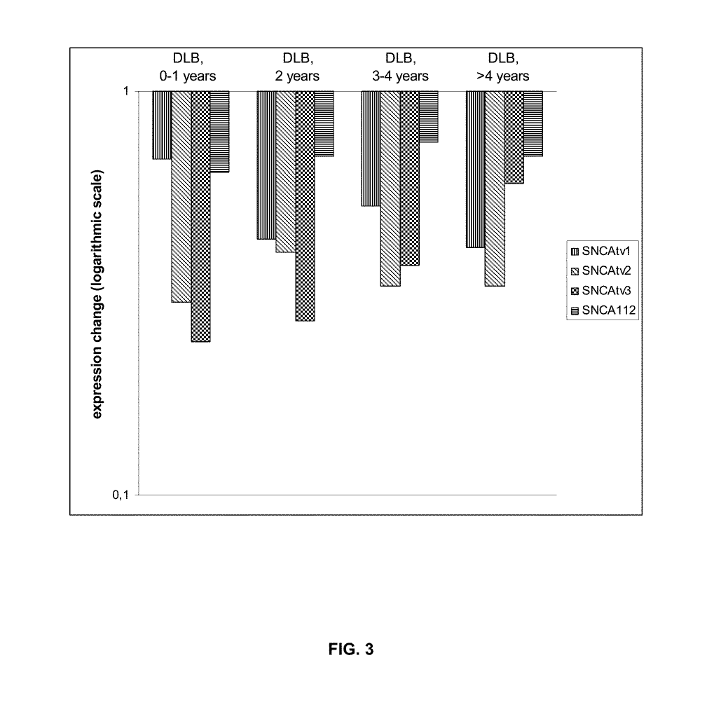

The results obtained are summarized in Table 5 and represented in the FIG. 3.

TABLE 5

| Time of | | | | |
|---|---|---|---|---|
| diagnosis | | | | |
| (years) | SNCAtv1 | SNCAtv2 | SNCAtv3 | SNCA112 |
| between 0 | 0.68 | 0.3 | 0.24 | 0.63 |
| and 1 year | (0.64-0.72) | (0.23-0.40) | (0.21-0.28) | (0.35-1.13) |
| 2 years | 0.43 | 0.4 | 0.27 | 0.69 |
| | (0.34-0.54) | (0.36-0.45) | (0.23-0.32) | (0.48-1.05) |
| between 3 | 0.52 | 0.33 | 0.37 | 0.75 |
| and 4 years | (0.41-0.66) | (0.23-0.46) | (0.35-0.40) | (0.73-0.79) |
| more than 4 | 0.41 | 0.33 | 0.59 | 0.69 |
| years | (0.29-0.57) | (0.32-0.34) | (0.50-0.68) | (0.41-1.16) |

As in the case of Example 1, the data shown in Table 5 were significant:
In the case of decreased expression compared to control subjects, the maximum value of the confidence interval was less than 0.5; and
In the case of increased expression relative to control subjects, the minimum value of the confidence interval was greater than 1.5.

Both Table 5 and FIG. 3 show that the transcripts SNCAtv2 and SNCAtv3 showed, in virtually all cases, a significant reduction in their expression in different groups of patients with DLB compared to control subjects. The only exception was observed in the group of patients with DLB diagnosed more than four years ago. In this group the SNCAtv3 transcript showed no significant variation in expression respect to control subjects, but a clear trend of reduction of expression relative to said control subjects. Therefore, the expression of transcripts SNCAtv2 and SNCAtv3 is reduced in patients with DLB regardless of the time elapsed since diagnosis.

In contrast, it was observed that transcripts SNCAtv1 and SNCA112 did not show significant differences significant in their expression between patients with DLB and control subjects.

The sensitivity and specificity of SNCAtv2 and SNCAtv3 transcripts for the diagnosis of DLB were above 90% in all groups studied.

### Example 4. Influence of Disease Progression on the Expression of SNCAtv1, SNCAtv2, SNCAtv3 and SNCA112 Transcripts

Data of variation of expression of transcripts SNCAtv1, SNCAtv2, SNCAtv3 and SNCA112 in patients with DLB normalized versus control subjects obtained in Example 1 were divided into four groups according to time of disease progression: 0 to 1 year (6 patients), 2 years (9 patients), among 3 and 4 years (7 patients) and more than 4 years (6 patients).

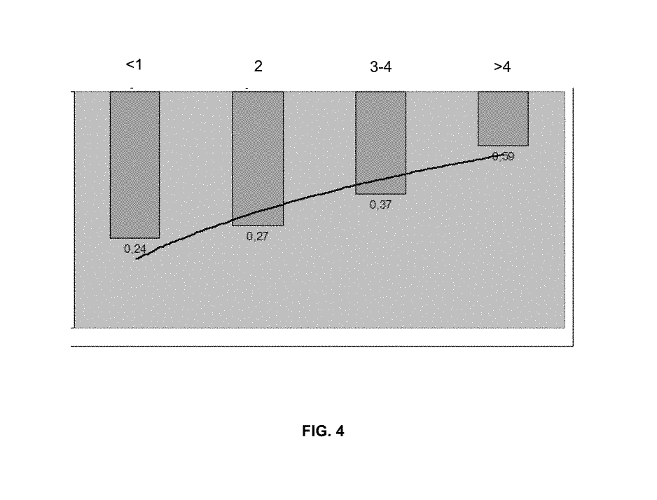

The results obtained are summarized in Table 6 and represented in the FIG. 4.

TABLE 6
Expression of transcripts SNCAtv1, SNCAtv2, SNCAtv3
and SNCA112 in patients with DLB normalized versus
control subjects during disease progression

| Disease | | | | |
|---|---|---|---|---|
| progression | | | | |
| (years) | SNCAtv1 | SNCAtv2 | SNCAtv3 | SNCA112 |
| between 0 | 0.68 | 0.3 | 0.24 | 0.63 |
| and 1 year | (0.64-0.72) | (0.23-0.40) | (0.21-0.28) | (0.35-1.13) |
| 2 years | 0.43 | 0.4 | 0.27 | 0.69 |
| | (0.34-0.54) | (0.36-0.45) | (0.23-0.32) | (0.48-1.05) |
| between 3 | 0.52 | 0.33 | 0.37 | 0.75 |
| and 4 years | (0.41-0.66) | (0.23-0.46) | (0.35-0.40) | (0.73-0.79) |
| more than 4 | 0.41 | 0.33 | 0.59 | 0.69 |
| years | (0.29-0.57) | (0.32-0.34) | (0.50-0.68) | (0.41-1.16) |

As in the case of Example 1, the data shown in Table 5 were significant:
In the case of decreased expression compared to control subjects, the maximum value of the confidence interval was less than 0.5; and
In the case of increased expression relative to control subjects, the minimum value of the confidence interval was greater than 1.5.

Both Table 6 and FIG. 4 show that the transcripts SNCAtv2 and SNCAtv3 showed, in virtually all cases, a significant reduction in their expression in different groups of patients with DLB compared to control subjects.

Additionally, when correlating with the progression of DLB, it can be observed that the levels of SNCA transcript tv3 correlate inversely with the advance of the disease. FIG. 4 also shows the results: blood obtained from patients who debuted recently with DLB contains lowest SNCAtv3 levels, which increased with the progression of DLB. SNCAtv3 levels remained significantly diminished but were increasing year by year of disease duration until disease duration of 4 years. Patients who were suffering from DLB for more than 4 years did no longer present significantly diminished SNCAtv3 levels in blood. The tendency is furthermore represented by the trend line overlaid in the graph (FIG. 4).

It is known that alpha-synuclein expresses specifically in neurons and that its aggregation is a key event in the development of neuropathological changes in synucleinopathies. It is also known that the development of neuropathological changes begins much earlier than symptoms become evident, namely in preclinical stages of the disease and that disease progression is associated with neuron loss. It is further known that at advanced stages of disease only a few neurons remain intact.

The above observations strongly indicate that: 1-alpha-synuclein aggregation rate is increased at preclinical stages of synucleinopathies, 2-alpha-synuclein aggregation rate is also increased during the first stages of disease, and 3-alpha-synuclein aggregation rate diminishes when disease is advancing due to increasing neuron loss.

Taken together, the aforementioned observations and the results on SNCAtv3 expression levels in blood of DLB patients suggest that: 1-SNCAtv3 levels in blood inversely correlate with the alpha-synuclein aggregation rate in brain, 2-determination of SNCAtv3 levels in blood may serve to detect pre-clinical alpha-synuclein aggregation events in the brain and 3-determination of SNCAtv3 levels in blood may serve to monitor alpha-synuclein aggregation rates in the brain, for example after the treatment with alpha-synuclein anti-aggregants in a clinical trial.

### Example 5. Study of the Expression of Transcripts SNCAtv1, SNCAtv2, SNCAtv3 and SNCA112 in an Additional and Independent Cohort of Patients Diagnosed with DLB and Control Subjects (Control Subjects Did not Present any Neurological Symptomatology)

To validate results from examples 1-4, expression of four transcripts of the alpha-synuclein gene was analysed: SNCAtv1, SNCAtv2, SNCAtv3 and SNCA112, in additional control subjects and patients diagnosed with DLB. For this purpose blood samples of 22 control subjects and 48 DLB patients were collected. The clinical diagnosis of the individuals was conducted in the Departments of Neurology of the Hospitals Germans Trias i Pujol and Bellvitge (Spain).

The methodology used for the determination of the four transcripts of the SNCA gene was performed as described in example 1. The same RNA purification methods, reverse transcription, primers and QPCR were used and data analysis was also carried out by using the methodology ΔΔCt.

TABLE 6

| | SNCAtv1 | SNCAtv2 | SNCAtv3 | SNCA112 |
|---|---|---|---|---|
| DLB patients | 0.59 | 0.48 | 0.42 | 0.88 |
| (expression | (0.53-0.69) | (0.42-0.55) | (0.35-0.48) | (0.61-1.22) |
| variation of | | | | |
| the transcript) | | | | |

As in the case of Example 1, the data shown in Table 4 were significant:
In the case of decreased expression compared to control subjects, the maximum value of the confidence interval was less than 0.5; and
In the case of increased expression relative to control subjects, the minimum value of the confidence interval was greater than 1.5.

Table 6 shows that transcripts SNCAtv2 and SNCAtv3 showed a significant reduction of expression in patients with DLB. However, it was noted that neither transcript SNCAtv1 and nor SNCA112 showed significant differences in expression between patients with DLB and control subjects.

The sensitivity and specificity of SNCAtv2 and SNCAtv3 transcripts for the diagnosis of DLB were above 90% in all groups studied.

Data of variation of expression of transcripts SNCAtv1, SNCAtv2, SNCAtv3 and SNCA112 in patients with DLB normalized versus control subjects were divided into three groups depending on the age of DLB beginning of the patients: before an age of 65 years (4 patients), between 65 and 74 years (16 patients) and after 75 years (12 patients). The results obtained are summarized in the following Table 7.

TABLE 7

| | SNCAtv1 | SNCAtv2 | SNCAtv3 | SNCA112 |
|---|---|---|---|---|
| DLB patients | 0.67 | 0.33 | 0.22 | 0.70 |
| who debuted | (0.53-0.87) | (0.27-0.39) | (0.21-0.24) | (0.47-1.06) |
| with disease | | | | |
| before age of | | | | |
| 65 years | | | | |
| DLB patients | 0.70 | 0.48 | 0.44 | 0.74 |
| who debuted | (0.58-0.85) | (0.47-0.49) | (0.42-0.46) | (0.46-1.21) |
| with disease | | | | |
| between 65 | | | | |
| and 74 years | | | | |
| DLB patients | 0.68 | 0.51 | 0.46 | 1.04 |
| who debuted | (0.59-0.81) | (0.42-0.64) | (0.41-0.50) | (0.65-1.57) |
| with disease | | | | |
| after 75 years | | | | |

Table 7 shows that transcripts SNCAtv2 and SNCAtv3 showed, in virtually all cases, a significant reduction of expression in DLB patients compared to control subjects. The only exception was observed in the group of patients with DLB debut aged after 75 years. In this group, the transcript SNCAtv2 showed no significant difference in its expression relative to control subjects, but a clear trend to reduced expression compared with control subjects. Therefore, expression of the transcripts SNCAtv2 and SNCAtv3 is reduced in patients with DLB independently on age of onset of the disease. Instead, it was observed that transcripts SNCAtv1 and SNCA112 showed no significant differences in expression between patients with DLB and control subjects in either group.

Both the sensitivity and specificity of SNCAtv2 and SNCAtv3 transcripts for the diagnosis of DLB were above 90% in all groups studied.

Data of variation of expression of transcripts SNCAtv1, SNCAtv2, SNCAtv3 and SNCA112 in patients with DLB normalized versus control subjects were divided into four groups according to time since diagnosis: 0 to 1 year (6 patients), 2 years (9 patients), among 3 and 4 years (7 patients) and more than 4 years (6 patients). The results obtained are summarized in Table 8.

TABLE 8

| Time of | | | | |
|---|---|---|---|---|
| diagnosis | | | | |
| (years) | SNCAtv1 | SNCAtv2 | SNCAtv3 | SNCA112 |
| between 0 | 0.77 | 0.37 | 0.32 | 0.63 |
| and 1 year | (0.72-0.83) | (0.28-0.46) | (0.28-0.36) | (0.32-1.13) |
| 2 years | 0.54 | 0.45 | 0.37 | 0.69 |
| | (0.44-0.66) | (0.42-0.49) | (0.36-0.39) | (0.46-1.05) |
| between 3 | 0.70 | 0.38 | 0.45 | 0.76 |
| and 4 years | (0.51-0.86) | (0.27-0.49) | (0.40-0.51) | (0.73-0.79) |
| more than 4 | 0.48 | 0.43 | 0.60 | 0.69 |
| years | (0.38-0.59) | (0.40-0.47) | (0.52-0.69) | (0.41-1.16) |

As in the case of Example 1, the data shown in Table 5 were significant:
In the case of decreased expression compared to control subjects, the maximum value of the confidence interval was less than 0.5; and
In the case of increased expression relative to control subjects, the minimum value of the confidence interval was greater than 1.5.

Table 8 shows that the transcripts SNCAtv2 and SNCAtv3 showed, in virtually all cases, a significant reduction in their expression in different groups of patients with DLB compared to control subjects. The only exception was observed in the group of patients with DLB diagnosed more than four years ago. In this group the SNCAtv3 transcript showed no significant variation in expression respect to control subjects, but a clear trend of reduction of expression relative to said control subjects. Therefore, the expression of transcripts SNCAtv2 and SNCAtv3 is reduced in patients with DLB regardless of the time elapsed since diagnosis.

In contrast, it was observed that transcripts SNCAtv1 and SNCA112 did not show significant differences significant in their expression between patients with DLB and control subjects.

The sensitivity and specificity of SNCAtv2 and SNCAtv3 transcripts for the diagnosis of DLB were above 90% in all groups studied.

### REFERENCES CITED IN THE APPLICATION

EP2539461

WO2011104696

WO201069603

WO9950300

### CLAUSES

1. A method for the in vitro diagnosis of a synucleinopathy in a human patient comprising the step of determining the amount of at least one transcript of the human alpha-synuclein gene (SNCA) selected from the group consisting of SNCAtv3 (SEQ ID NO: 3) and SNCAtv2 (SEQ ID NO: 2) in a biological sample obtained from the patient.
2. The method according to claim 1, wherein when the amount of the transcript(s) determined for the patient is reduced with respect to a reference value, this is indicative of the presence of a synucleinopathy in the patient.
3. The method according to any of the claims 1-2, wherein the amount of SNCAtv3 transcript is determined.
4. The method according to any of the claims 1-3, wherein both SNCAtv2 and SNCAtv3 transcripts are determined.
5. The method according to claim 4, wherein the synucleinopathy is dementia with Lewy bodies.
6. The method according to any of the claims 1-5, wherein the step of determining the amount of SNCA transcript is performed by quantitative real-time PCR using primers with SEQ ID NO: 6 and SEQ ID NO: 8 for determining SNCAtv2 and primers with SEQ ID NO: 7 and SEQ ID NO: 8 for determining SNCAtv3.
7. The method according to any of the claims 5-6 that additionally comprises determining at least one other marker known as being indicative of dementia with Lewy bodies.
8. Use of means for determining the amount of at least one transcript of the human alpha-synuclein gene selected from the group consisting of SNCAtv2 and SNCAtv3 in a biological sample for the diagnosis of a synucleinopathy in the method as defined in any of the claims 1-6.
9. The use according to claim 8, wherein the means include at least one pair of primers selected from SEQ ID NO: 6/SEQ ID NO: 8 and SEQ ID NO: 7/SEQ ID NO: 8.
10. The use of any of the claims 8-9, wherein the means form part of a kit.
11. Use of at least one human alpha-synuclein gene transcript selected from SNCAtv2 and SNCAtv3 for the diagnosis of a synucleinopathy.
12. Use of SNCAtv2 and SNCAtv3 alpha-synuclein gene transcripts for the differential diagnosis of dementia with Lewy bodies.
13. Use according to claim 12 in combination with at least one other marker known as being indicative of dementia with Lewy bodies.
14. A method to stablish the response of a patient which has been diagnosed with a synucleinopathy to a medical regime for the treatment of a synucleinopathy, which method comprises determining the amount of at least one human alpha-synuclein gene transcript selected from the group consisting of SNCAtv3 and SNCAtv2 in a biological sample obtained from the patient being treated and comparing said amount of transcript(s) with the amount of transcript(s) determined for the same patient before the treatment or at an earlier phase of the treatment, wherein an increase of the amount of transcript(s) with respect to before the treatment or earlier phase of the treatment is indicative of a good response to the medical regime.
15. The method according to claim 14, wherein the amount of SNCAtv3 transcript is determined.

1. A method for the in vitro diagnosis of dementia with Lewy bodies in a human patient comprising the step of determining the amount of transcripts SNCAtv3 (SEQ ID NO: 3) and SNCAtv2 (SEQ ID NO: 2) of the human alpha-synuclein gene (SNCA) in a biological sample obtained from the patient, wherein when the amount of both transcripts determined for the patient is reduced with respect to a reference value, this is indicative of the presence of dementia with Lewy bodies in the patient.

2. The method according to claim 1, wherein the step of determining the amount of SNCA transcript is performed by quantitative real-time PCR using primers with SEQ ID NO: 6 and SEQ ID NO: 8 for determining SNCAtv2 (SEQ ID NO: 2) and primers with SEQ ID NO: 7 and SEQ ID NO: 8 for determining SNCAtv3 (SEQ ID NO: 3).

3. The method according to claim 1, which is for the differential diagnosis of dementia with Lewy bodies with respect to Parkinson disease.

4. The method according to claim 1 that additionally comprises determining at least one other marker known as being indicative of dementia with Lewy bodies.

5. Use of means for determining the amount of at least one transcript of the human alpha-synuclein gene selected from the group consisting of SNCAtv2 (SEQ ID NO: 2) and SNCAtv3 (SEQ ID NO: 3) in a biological sample for the diagnosis of dementia with Lewy bodies in the method as defined in claim 1.

6. The use according to claim 5, wherein the means include the pair of primers SEQ ID NO: 6/SEQ ID NO: 8 and SEQ ID NO: 7/SEQ ID NO: 8.

7. The use of claim 5, wherein the means form part of a kit.

8. (canceled)

9. (canceled)

10. A method of deciding or recommending to initiate a medical regime for the treatment of dementia with Lewy bodies in a patient comprising the steps of:
(a) determining the amount of SNCAtv2 (SEQ ID NO: 2) and SNCAtv3 (SEQ ID NO: 3) transcripts in a biological sample obtained from the patient, and
(b) comparing the level obtained in step (a) with a reference value,
wherein if the amount of both transcripts detected in step (a) is lower than the reference value it is indicative that the subject would benefit from a medical regimen for the treatment of dementia with Lewy bodies.

11. The method according to claim 10, wherein the medical regime which is recommended comprises administrating acetylcholine esterase inhibitors.

12. A method to stablish the response of a patient which has been diagnosed with dementia with Lewy bodies to a medical regime for the treatment of dementia with Lewy bodies, which method comprises determining the amount of human alpha-synuclein gene transcripts SNCAtv3 (SEQ ID NO: 3) and SNCAtv2 (SEQ ID NO: 2) in a biological sample obtained from the patient being treated and comparing said amount of transcripts with the amount of the transcripts determined for the same patient before the treatment or at an earlier phase of the treatment, wherein an increase of the amount of both transcripts with respect to before the treatment or earlier phase of the treatment is indicative of a good response to the medical regime.

13. The method according to claim 10, wherein the step of determining the amount of SNCA transcript is performed by quantitative real-time PCR using primers with SEQ ID NO: 6 and SEQ ID NO: 8 for determining SNCAtv2 (SEQ ID NO: 2) and primers with SEQ ID NO: 7 and SEQ ID NO: 8 for determining SNCAtv3 (SEQ ID NO: 3).

14. The method according to claim 10, wherein the medical regime which is recommended comprises administrating acetylcholine esterase inhibitors and wherein the step of determining the amount of SNCA transcript is performed by quantitative real-time PCR using primers with SEQ ID NO: 6 and SEQ ID NO: 8 for determining SNCAtv2 (SEQ ID NO: 2) and primers with SEQ ID NO: 7 and SEQ ID NO: 8 for determining SNCAtv3 (SEQ ID NO: 3).

15. The method according to claim 12, wherein the step of determining the amount of SNCA transcript is performed by quantitative real-time PCR using primers with SEQ ID NO: 6 and SEQ ID NO: 8 for determining SNCAtv2 (SEQ ID NO: 2) and primers with SEQ ID NO: 7 and SEQ ID NO: 8 for determining SNCAtv3 (SEQ ID NO: 3).

16. The method according to claim 12, wherein the medical regime comprises administrating acetylcholine esterase inhibitors.

17. The method according to claim 12, wherein the medical regime comprises administrating acetylcholine esterase inhibitors and wherein the step of determining the amount of SNCA transcript is performed by quantitative real-time PCR using primers with SEQ ID NO: 6 and SEQ ID NO: 8 for determining SNCAtv2 (SEQ ID NO: 2) and primers with SEQ ID NO: 7 and SEQ ID NO: 8 for determining SNCAtv3 (SEQ ID NO: 3).


SEQUENCE LISTING (10 sequences)

SEQ ID NO: 1 - DNA, 121198 bp, Homo sapiens
gcacatacaactttaacttcaatattttaatgacgaaatttaaggataatttaaatagaaatggactcagaaaagaatcagtaagacttagtgaaggatcattgtctattatagagaagttgatttaagattaacttattagtaatatttaacatatataaagaattattagactgggtatatagacaagcgttttattcttggaagacaaaaagaagaaaaattgaattcaaccgatgtatacgaaaataaaaagtaacagtaaattaaaaatagataattaaataaatatatgatacagtataacgttttatagccaagatgatgttacaaatccatatttattgacatggatatgtttttatactaaagtgtttatcaaatagccattaagagataacttctttgaataatttgctttctaaatttcttaactacataaatttccagctttatatggaacaccaagttttcaaaccattagtgatgtgctttttatatggtgttaaaaagtttctttctttcttttttctttttcccccaagatggagtcttgctctgtcgcccaggctggagcgcagtagtgcgatctcggctcagtgcaacaaccacctcctgggtacaagcaattctcctgcctcagccccccaagtagctgggattacaggcacctgccaccacgtccagctgatttttgtatttttagtagagacggggttttaccatcttggccaggctggtctctaactcctgacctcaggtaatctgcccacctcagcctcccaaagtgctgagattacaggcgtgagccaccatgcccgacctaaaaagtttcttaaacgtcactttatactctcaaattatctagaaaggaaaacgtattagattcctggatattttggatattgtaaggaacatacttatttgctgtatatactctgtttgtaacagtattgtaacttcagttcaaaacaatacacaaaacattacaagttcccgtgatattttaaaaattcatttattttcttcctttctgaatacaaatgctgttcagtctgttgattcttcactaatctgaaatattagggactgatttctgaattggatattcattctgaagcctttcagagccactggcacaaagggtctgtcaaacttggaacaccatttgttgtatcattttatttttttctcttggcaaatccacataattcatacaggactatgccagtgtcttttgaaagaaacaaggtttaagaaagtaaaaatgttaataaagatagtgaatgttaattctgtcattgttactgtatttcttcaagctgtggctgcaaactgctttgagtgatgttattgtaactcgcacattagggagagaaagagatgtttggtagatttttaattaatgatccctatcaatgctccttgagctttcccactctatctctccacaacttccatccctggttggaaattttttgcttacccatactaagtgagagttattgatgggaaggcatcagatatctcacgtgtgttgctggtgggatgggagactgtggaggatgggaacaggtggaaatctactgcaatggaaaaaaaaaaaaagcatgtcctaggacacccaaaacatggaggctagataataacaatagctacttgtactgagagcttccactctgcctggctctttgctatgagccacattattcattccttacaacaatcaaacaagacaagtaaaatatcatgcccattttttaatgagaaaactagagattagagaggttatagatacttgctctgagtcactagtaatgagtagtagagctttaataagtccctgaatttaggttgtatctagtacatttactcttagaagtctatcatgctcaccagagttgcagagttgcgtgtatttcttgggctcattaatgtgtttttttctttctaaaactaaagtcatttgaacttgttagattttgaaatatttaaatatcttttctatctggctttaacatctttaatcttggaatcttgcatgccttcatattcttaggaccacgaaaccacaggaatatttaaaatgatatctagtggaaacaatatgaagttggccatggggtcaaattagagaatctgaatactatgcttctccttgattgctcttcccatttcttcagagtaaccctattcccccatctcatgctcaccccctttccaaaatcatacataatgatctcccaacagtatgcattaggctttctctactctacccactatgaaattacacaagaagcctatcgcaatctcactacctcgtctctctcacaggtttacagaaggtgagaggaaggtgcagatagagaataagaagcaggtggctccagcatcaacattacatcaccccttgtgttcacaacaaatacggaatattatccaaagataataaacgttgtattttcttaacttaaacacattaaatcagtcctctctttaatcaattgttaatgggcagcatctttattttcatgccattctactctgctgtctttgctatagcacaagtttaccacataccatacctaaaaattcagttgttctatgggggtaaacaaagtctaggttaagcatatatttcatagaatgttaatctatagcaaaattaatgaattaaatccagataaaagaatcctattatggtctggtaaaatatttatatttcacttagcaaagagaaaacaaaacatgaatattgtagttatgaacagaatatgcatgttagtaatgcttccaaatatgttattacttcataacttcatatttcttatgaggtacaagccattcaattagtttaacgttatattcagagaggctaaagatttactgaagaccatgctgtccatcaataatgaaaagaaaaattaaaaaaactttattttaacttctagttcccttctttgtacttgagcagctttccctccttaagaatacagacctagaacatatgcaatatcactatcaatattatgtgtaattaaaagttcattggatgtttactgtgttcaaggcattttaaggagtgacaagagttaaacatatagttgtaattcaaaatgacaacgaaattagtttacagttttctttttttgtaggtagtaagaaatcatctccccctattgaggaataccaatatagaaaaggcaaaactttaaatatgaatgaactgtttcataataacataagttcttcttgatttccattgtcacatccaaatttgaaggctatttctaacacagctgggttctacctttttccttctcactctttaccacacccaatctgtgaggcttcagacacaaactgctaattcaggagacaattgtgccttctgtaacagtttctgctaaattgtctcagctctgccacttaaaatagctaggtgatctcagcatatcaccaaaactcttggagctcagtttctctgtctataaaagttacataaaatgtaattgatctgcttgttatgactaaataacatagtacattagtcctttgccaaaggactaacaaattaccaaataaaagtttggaatcatgttaaacgtttataagaagtgcaactgtccagaaataattctctcacattggtctgttgtaatgagacctaaaatatctcattttatttacctctttgacttaaagcactaggtctcaaggaggtcatggttatactataaatatgtcatgtgaaataatatattaaataattgttgtaatactctattgagatactagttgtaaagaggcacaatggaaaacttatactattaacagtagtaaaaagaaacaacaaaaagcaataaaaaacaaaacacccattcatgcaacgacatgaacgaacctcacaaatattatactgagtaaaagaagtcagacaaatataaaacaaagtttatactacgtgattagatctttatgacattctagaatatgcacatgaaggtacaaggtaactgtctggaatgatgaaaatgtcctgtgtcttcaaaatagtgtgggttacactaatgcatggctttttcaaaactgatttaaagggacacaacatctgagcatttccctaggtgtaaattacactgcaattttaaagaatcatctaatgatattgtggttatttttaaacagtccttaaattttgtggatgcatactgaatgtttacagctgaaaagatatatataaagcttgaatttggtaaaaaaaaaaaaaaaagagggaggattggtagtgataaagtgagtggacttatggatgagacatgatcagccatgcattgaaaaaatgtaaaagttggatgatcttcacatgagagtcctttattctgtctacttttgcatatgtttgaatatttcccataacaaaaagttgaaaatagagtgatcacatgagttaatctcctaatttacaaaaaagaaaactggaaacagaaggagaacaaaacttgttcaaggtctcaaagccagacagcaaactagctcccaagtccaaccttcttgctctggtcctaagcaaacaaaaaatattaatatgagctactgcattaaggaaagtctgcttttccaaagggcagaccaatagttcaaggaagagtttaaataataaatatttgtgatcttactttcatgcttttctattttccactgaacacatatgcattatcttctatatgtcttttatgtataatcatttgcttcctgttccttgtggttttaaagttgttttgtatgtttaaatttgattttactcaaatttcagaacccaaattagcgcaagaatcagacaaagcataactttctataaatataaaaacaattaaaaaaaaaacatacagcaaaaacgagttgttgtttcccccctcctcttccagtgcttaactaatcttccgaatccaggcacagaaagcaaaggctttctgctagtgggaggagcttgcttctccattctggtgtgatccaggaacagctgtcttccagctctgaaagaggtgaaaatgtgttaagcgatgcaaaaattgtcttgaagttcgcgtgtgtatgtctgtgtgcatgtgcgtgtggtgggtggggggagagaaaagggggtgtcaattctgagggcaacgagaatcagaagtcagaaaggtgagtggtgtgtagcatctccctttcagaaggggctgaagaagaaattggatatgatggtccggtaggctaaatcacgctggatttgtctcccagataaagggaggtctgcaaagtaagtcccatttctagagcgaaaagccttaggaccgcttgttttagacggctggggaatatttattccttgttccactgatgggaaaatcagcgtctggcaggcgctgattggtggaaaggaaaatggtgatagtggcgtggaaagaggatttgctgagccttctcctgcctcctcaacctgtgactcttccttagtagtctccctttcaccctcaggaccctttccggctcttcctagattaagagcaaacgaaaaccttgaagatatttgaactaaagcgacccctaacgttgtaacctgtgaccgtgattaaatttcagcgatgcgagggcaaagcgctctcggcggtgcggtgtgagccacctcccggcgctgcctgtctcctccagcagctccccaagggataggctctgcccttggtggtcgaccctcaggccctcggctctcccagggcgactctgacgaggggtagggggtggtccccgggaggacccagaggaaaggcggggacaagaagggaggggaaggggaaagaggaagaggcatcatccctagcccaaccgctcccgatctccacaagagtgctcgtgaccctaaacttaacgtgaggcgcaaaagcgcccccactttcccgccttgcgcggccaggcaggcggctggagttgatggctcaccccgcgccccctgccccatccccatccgagatagggacgaggagcacgctgcagggaaagcagcgagcgccgggagaggggcgggcagaagcgctgacaaatcagcggtgggggcggagagccgaggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcaggtaagtacctgtggatctaaacgggcgtctttggaaatcctggagaacgccggatgggagacgaatggtcgtgggcaccgggagggggtggtgctgccatgaggacccgctgggccaggtctctgggaggtgagtacttgtccctttggggagcctaaggaaagagacttgacctggctttcgtcctgcttctgatattcccttctccacaagggctgagagattaggctgcttctccgggatccgcttttccccgggaaacgcgaggatgctccatggagcgtgagcatccaacttttctctcacataaaatctgtctgcccgctctcttggtttttctctgtaaagtaagcaagctgcgtttggcaaataatgaaatggaagtgcaaggaggccaagtcaacaggtggtaacgggttaacaagtgctggcgcggggtccgctagggtggaggctgagaacgccccctcgggtggctggcgcggggttggagacggcccgcgagtgtgagcggcgcctgctcagggtagatagctgagggcgggggtggatgttggatggattagaaccatcacacttgggcctgctgtttgcctgagtttgaaccacaccccgagtgagcagttagttctgttgcctacgcctttccaccatcaacctgttagccttcttctgggattcatgttaaggatacccctgaccctaagcctccagcttccatgcttctaactcatactgttaccctttagaccccgggaatttaaaaaaggggttaatcttttcatgcaactccacttctgaaatgcagtaataacaactcagaggattcatcctaatccgtggttaggtggctagacttttactagccaagatggatgggagatgctaaatttttaatgccagagctaaaaatgtctgctttgtccaatggttaaatgagtgtacacttaaaagagtctcacactttggagggtttctcatgatttttcagtgttttttgtttatttttccccgaaagttctcattcaaagtgtattttatgttttccagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggtaggtaaaccccaaatgtcagtttggtgcttgttcatgagtgatgggttaggataatcaatactctaaatgctggtagttctctctcttgattcatttttgcatcattgcttgtcaaaaaggtggactgagtcagaggtatgtgtaggtaggtgaatgtgaacgtgtgtatttgagctaatagtaaaaaatgcgactgtttgcttttccagatttttaattttgccctaatatttatgactttttaaaaatgaatgtttctgtacctacataattctatttcagagaacagttttaaaaactcatagtcttttaaaaaataatcaagaatattcttaagaatcaaaatcattgatggatctgtgatttcttttaccatcatgaaaaatgtttgtcaattttaatccattctgatttttaaaatatgactttgatatgcccctgtgatgtgtataaagagacctatttgtggccctaaaatggaaagaacagattagtctttgatagagttacttcatgtgatcatttggtctctgtgaacactgaggacagagaaaagtgcttgagggctgctactaatctctcagaaacatttgtatagttcatccatcaaatgacacacatactaaaagaataaagaaattgatgcttattacctacttgttcctaaagttccaccttggggtatacacccaaactctgactctcttttctgtaacttgaactgtattcaattgagtgttattttacaaaccactttgaattccttggaaaagaatagacacacactctcatccacaggcatagacacacacactcaacacagacacattgcccattcttcctctcttctttctcctctgagctttttcacattctctggtggcaactatagcagtaagagtcacaggatgaacagtcaggtggaggatgaccacattgagttgcctagctgaaacatgtgctccgtctatgtctgcaaagtgaaagaaagctacactatctcttcaacatagatcagtgggggaaattttatacttgggatgatttatatgaatgcatctcatcaaagttcacaacacatttttttttcagttttttattttcagtttttagagtcagggccttgctctgtcgcccaggctggactgcagtgatgctatcatagctcactgcatccttgaattcctgggctcaagtcatgcccccacctcagcctcctgagtagccaggattataggcatgtgccactgcctcattatttagacttttcttatgttgacttaatcttcccacaaatcttcaattaaattactttttttctaccttaaaacatattttcagaaagtcattgaaatagggtgttacaagaggaaaaaattgatgagttaattttaaatattttatgaagtgtgaattatacctttttagatggaatttggaatactgaatcagtgacatgcagtttatcaatatctttccgtttgtcctcagatttccaagttctgcaagcacaagtttctttgacttagttaccttttaactgttcattgaaatcattttcaatgtctctcatggcatttaacacatagcacattctataaattttttattggttacattctgagttctaattgagagttgaacttacacacagaatttaagataaaaaatgaccatgtgaagacacaatagtatagtccagggattggcaaaattttgggtaaggaatcagatagcacgtattttaagccatgagatctatgtcttggccaggtgccgtggctcaggtctttaatcccagcactttgagagcccgaggctggtggatcacttgagcccaggggtttgagaccagcctgggccacatggtgaaaccctgtgtctacaaacaacgcaaaaattagccgggtatggtagcatgcatgtgtattgccagctacccaggaggctgaggtaggaggatggcttgagccatacagctcactgcagaggttgcagtgagctgagatcgagccactgcactccagcctgggtggcagagtgataccctgtctaaaaaaaagaaaaaaaaatctatgtctcaattctgctgttgaagtgtgaaggtagtcataaacaataactagtgtggctgtgtcccaataaaacttcatttatcaaaacaggtggtgggctggaattgtcttgtatgttgtagcttgctgactactgatagagtggaaagaacatgcactaatcacacaaaccaaagttttagttgagactacatcacttatcacctttagggtcttggggaagcgtacttaacatctctgagcatcacttccctgattagtaaaaaatatgatttagaaaactgcaactaccttgcagtttttgtgggaatgtcataataagacaggacatatgaataattgagcacacttttatatataggaaccatggttattattatcaaataaactctccaacggaataattactttgccaacacgttttccatttattcttttatccttcattacataactagtttgaaagattggaggcgaccaaagaccattttataatttcacttatggctgaagatgtttggtagaagcctcataagaaaagtaatctcattcctttataagaatatacttttaacaactactttttaactcattgaagaactaccttaatgatcagtgttatttttatgggttttgttccctccatttttgttatctgcgtacaccaattttcaatcaacatacttcaatttaatagacaaaaatttcttcaaatgactcagaaattaattagatctaaatccaaaagcagaaagatttaattatctttatataatgctcagtaatataaatgcaataaatacaagaaaatgatgatctttgagtgtcttccaatgccactctgctcaataagcagcagtggccatcagtgaaattgatagcaaattctcaagtcaaaatgtgcttcacctcactaagctgacaaagtcaacataacatgcacaacagggataactgagttctcaaaactctcaggtattacttctgaccttcttctccactctgtgctcttttgaggttgggaagacaagatagggtgtgtgtgggacacctccgctcagggaagccatcagctctggtgtccctacagcatttataccttgctagtcacataaccacttggcacctattttgtaggtgtacgttatcaattacagattactcataaattaaaggctaaccatcaattacagattattagtaaataattatgacctcaaagaacaactgattggtttgatacatggtaaccttatgaggactctcatttatctcgtttttttaagttatatacctatctctttggggttgcactacaaaaatataaaatatgttgcataagatatttataaaaaataattaattataagttctaatggtgtggtttagtggcattcttttttttttctttttttctgagatagggtctcaatctgtcatttcactccaggctgaagtgcagtggtgtgatctcggctcactgcaacctccgcctcctgggttcaagttattctcctgactcagcctcctgagtagctgaaattacaggcatgcaccaccatgcccggctaatttttgtatttttagtagagatggggtttcaccatgttagccaggatggtctcgaactcctgatctcatcatccccgacctcggcctcccaaaatgctgggattacaggcgtgagccattgcacccggcctagtggcattcttttttaaaaataaatttaattgtgtatatttagggtatgcaacatgatgctatcagatacattagacactaaaaaattactatattgaagcaaattaatatattcataatctctcatagttaccttttttgttgtttttgtggcaagggcagctaaaatccacttatttatcatgaatctcaaatatagtacaattttatcacctacagtcctcatacattagatctgtacactttttcatcttacacatctgctacttgcttggatcctatggcctatatgtccctattttctacctacttttccacccctattaaccctgttttttacgtagtctctgtatatttgaattttgtttcaagcttccacatatatgtgagataatgtaatatttttctttctgtgtttggcttatttcacttagcataattttgtctgggttcatccatgttgtaaatggtaggatcttgtttttttagggctgactgatattccattgtatctatgtaccacaatctttttatctacctatctatcagtagacactttagttgtggctattatgtttttctttttttcttttttggagacagggtcttgctgtcacccaggctgcaatggagtggtgttatcatagctcactgtaacctcaaacttctgggctcaagagatcctcctgccttggcctcccaagtagctgagactacaggcatacattaccatgcctggctaatttttaatattttttgtagatatagcatctcactctgttgcccagactggtctcaaactcctaattcaaatttagaatagagtatgacaattctgtaaaatataaaaaacatgtccactccgtataggaagttatacaatgagaagaagacaaacactatttacattactcttgataagttttttacaaagaaataaaacactttaatttctaatgttttaaattctggtttgctaaataaataaatattagttttagtgtttttaaaattccttatatagttataagtgatcttcctgcctcagcctcccaaagcactgggattccaagcaagagccactgtgttggggcccttggaaacagatatgctgaaatcttttcttgtggatctacacccagaagagggattgctgggtcatatgctactctatttttaatttttcttttatttttagtgaatatgtaataattgtatataattgtgggatccagaattatatttccatacatgtatacaatgtgtgataatcaaattagggtaattaacatatccattacctgaaacatttatcattcctttgtggtgggaacagtaaaaattaaaaattctctcttctagatttttgaacatatgcaataaactattgttaagtatatcaccctacagtactacagaatgctagaactcattcctcatatttggctccaatttcatattctttaaccaacctctccatatcctcccctccctcttaccgttgtcagcctctaataatcataattctactctctacttctatctcattgtctttgatttagaatatgtttcataatttaaccaaaggtcaaattcttaggtactgctaaggcaaagaacaaagatcgcattccagctgttagacatttcttactactagtcatttttaagacaacatggggtgcaggtggtgaggatgagagatagagattgaaacatattctcttaaatatcagctgttctcactctgcatagttccagcacaaacaaattccaggtactatggttagttaaataacaccagccactaacaacacaattcaaatttctgttaccacagtataccgaaagtcattgcataaagtacaaactttgctgctaactcttcagccttcaaatcattacataaataacagaaacccattataatcagtgacaaaaccacagcacttctttcaaagctttttggagattggttgcttcacatctgttatgcagttcatacagacagcaatgcccggacttgtgtggccacattgtctcccagtggtgagcccatgtgatgtttcacgaaaatgcgcaatcaaaagaggaaactggccagcaaagatgaaagagtagcaaacaaaggaagtgaaacattctggaagtaaaatttgaatcaaacataagttgatgtatacaggaagtagctaccctgaggatgttgtcactgctgcaattcaggagactctaaatatgcagtcagaggaacgtagtgaggtgaaggtatccgtataatggggaaagaggttgtgataaagagtgaaggtgtcccagaggaagtgttgctgaaaaatacaccttatgttaaatacactgtcagtatatcatgacattaaagtgcaaatgataacattttgtaaactgatccaaacttaaaaaggagtatgataattctgtaaaacataaaaatcatgccgattccataaattatacagtgtgaattacactgaaaaatccaacattagagaggatatgaatacaattttttacaagcataattttaataatacacataataattatttgtattcaagtttagtaatgttcaaggtttggaagaaattctgatcctgtgtagagaccctagtttgaatgtgcttatagcctattattacatgtgtaatgttacataaattacttaactcggatttttaatttcatcagctatttaaaatgggcataatataactatattaaatggctgttatgaagattaaataagatgatatgtaaaatgtgttttttgtttgtttgtttgtttgtctgtttgtttttttgagacagagtcttgctctgttacccaggctggagtgcagtggcacaatcttggctcactgcaagttctgcctcccgagttcatgccattctcctgcctcagcccctcccaagtagctgggactacaggcacccgccaccacgcctggctaattttttgtatttttggtagagatggggtttcaccatattagccaggatggtctcgatctcctgacctcgtgatctgcccacctcggcctcccaaattgctgggattacaggcatgagccactgcgcccagcctaaaatgttttttttacataatgggtgttcagcacatgttaaagccttctctccatccttcttcccttttgtttcatgggttgactgatctgtctctagtgctgtacttttaaagcttctacagttctgaattcaaaattatcttctcactgggccccggtgttatctcattcttttttctcctctgtaagttgacatgtgatgtgggaacaaaggggataaagtcattattttgtgctaaaatcgtaattggagaggacctcctgttagctgggctttcttctatttattgtggtggttactggagttccttcttctagttttaggatatatatatatatttttttctttccctgaagatataataatatatatacttctgaagattgagatttttaaattagttgtattgaaaactagctaatcagcaatttaaggctagcttgagacttatgtcttgaatttgtttttgtaggctccaaaaccaaggagggagtggtgcatggtgtggcaacaggtaagctccattgtgcttatatccaaagatgatatttaaagtatctagtgattagtgtggcccagtattcaagattcctatgaaattgtaaaacaatcactgagcattctaagaacatatcagtcttattgaaactgaattctttataaagtatttttaaataggtaaatattgattataaataaaaaatatacttgccaagaataatgagggctttgaattgataagctatgtttaatttatagtaagtgggcatttaaatattctgaccaaaaatgtattgacaaactgctgacaaaaataaaatgtgaatattgccataattttaaaaaaagtaaaatttctgttgattacagtaaaatattttgaccttaaattatgttgattacaatattcctttgataattcagagtgcatttcaggaaacacccttggacagtcagtaaaatgtttattgtatttatctttgtattgttatggtatagctatttgtacaaatattattgtgcaattattacatttctgattatattattcatttggcctaaatttaccgagaatttgaacaagtcaattaggtttacaatcaagaaatatcaaaaatgatgaaaaggatgataatcatcatcagatgttgaggaagatgaggatgagagtgccagaaatagagaaatcaaaggagaaccaaaatttaacaaattaaaagcccacagacttgctgtaattaagttttctgttgtaagtactccacgtttcctggcagatgtggtgaagcaaaagatataatcagaaatataatttatataatcggaaagcattaaacacaatagtgcctatacaaataaaatgttcctatcactgacttctaaaatggaaatgaggacaatgatatgggaatcttaatacagtgttgtggatatgactaaaaacacaggagtcagatcttcttggttcaacttcctgcttactccttaccagctgtgtgttttttgcaagattcttcacctctgtgtgatttagcttcctcatctataaaataattcagtgaattaatgtacacaaaacatctggaaaacaaaagcaaacaatatgtattttataagtgttacttatagttttatagtgaactttcttgtgcaacatttttacaactagtggagaaaaatatttctttaaatgaatacttttgatttaaaaatcagagtgtaaaaataaaacagactcctttgaaactagttctgttagaagttaattgtgcacctttaatgggctctgttgcaatccaacagagaagtagttaagtaagtggactatgatgccttctagggacctcctataaatatgatattgtgaagcatgattataataagaactagataacagacaggtggagactccactatctgaagacggtcaacctagatgaatggtgttccatttagtagttgaggaagaacccatgaggtttagaaagcagacaagcatgtggcaagttctggagtcagtggtaaaaattaaagaacccaactattactgtcacctgatgatctaatggagactgtggagatgggctgcatttttttagtcttttccagaatgccaaaatgtaaacacatatctgtgtgtgtgtgtgtgtgtgtgtgtgtgtgcgtgtgtgtgagagagagagagagactgaagtttgtacaattagacattttataaaatgttttctgaaggacagtggctcacaatcttaagtttctaacattgtacaatgttgggagactttgtatactttattttctctttagcgtattaaggaatctgagatgtcctacagtaaagaaatttgcattacatagttaaaatcagggttattcaaactttttgattattgaaaactttcttcattagttactagggttgaatgaaactagtgttccacagaaaactatgggaaatgttgctaggcagtaaggacatggtgatttcagcatgtgcaatatttacagcgattgcacccatggaccaccctggcagtagtgaaataaccaaaaatgctgtcataactagtatggctatgagaaacacattgggataaatcggctgctatcataatcattcctctcccacatcagataaatgaattaactttttgaatagggttatttaatataaagtgcttaagtctaattatgagaagaaataagataattacacttcaatggttaaagagagggagaataatttgcatattatgcctgatgtaaaatgtttattatgggtacatattaagtgctaactaattgttaattgttcttgctacaagtcttaatgcagggaaacaagaaattattacatagtacctaatattatcttctaatattaaagaaacaatttcccctaaattcatcccattagctttttttttttcggtggggcaggggagaaatacagacttcagtaaacttgggctgggaactttctacctacaaagttcaaataaaataaattatcctagttagataatatcaatgaaaaatccaccaacttaaatcctggctgtttgatctcaggaaattatttcagttatcaacttaatgcatcatattatagaaatatatgaaaatgtgtttaattaaacttactgaatgatatgttttttcaggtactttaaaaataaactatgatataaagttacctatttttcatgcaagtatagtataaagaaatttctaacactggagattttctgaaggttttgattcttataaatttattacatcataatgaacaaaactaattttcaacatattatgatttaaatttccttagtaaattgtttcaaatttattttctttaaatccatatttacatatgtatatttaaatatacatatttacttgtataacaattcaaaaccatatattaattttataattttgtttaatgtcaaaggttagatttggctatatctattctaaaagttggtatcacatttcctttttggaattttatttttaaagtagctaaagtcaaatataaacctattatttatattaatgcagacattagaggtagacactaaattcattttagtatattctaaattatttattatctactatgaaataatataaagaaaaataaagcagaatccctgatttcaaagaactcaattgccgaaaaacagttaccatttattagacccaaaatgtactaatatgagtgtgtctcttttccttttgttttgtcacccgtcatttggaatgtcagtgagtagagagatagtgtgaaaggccctcaaggggaaaaatagaggttaaaggtcagcagagaccctactagagaaatcagttctacagaaatgtttttaaatgtgtcgattattgctacatgtacactctgtcattttgtaatgtagccattttatttatgattataataataaaacaacaaaattataataatgtgtagagtacattttactgtgcagtgtattgcattaaaactagattaaaatttatacatatataaaaggctatctagatattataaaatttatggctggatctgtaaaaaattcaaaacctatttttaatctcgctttgagattttataacaagaaaatgttcgtttcaagcaaaattttcaattcacgtccttgaaaaggaaaaaaatgacaacttgaaacacataattgactatttttaaaggatcaacatttcagaaatgttttaaaacataagattttcagtacagcttttcgctggcatttaaatcgaactttgaattgtaaatagctcttgctcttaaggagacatcagccatatccttagaagtggcacggagttgttaggtagttgtacaaaattctagcctaaaagacaaatagggagcaacactactgtggaccgtttctggtcttgggctgtgtggctatgtcaggcttgcccacattgcctgtactaaggagaaagcctcttgtccttacagacccccttagcttacatagtctatttgaaaacaaattgctttgtccacaccatttaaatattggcttcaggccaggcgcggtggctcacgcctgttatcccagcactttgggaggctgaggcgggcagatcacgaggtcaggagatcgagaccatcctggctaacacggtgaaaccctgtctctactaaaaatataaaaaaattagccgggtgtggtggcgcgcacctgtagtcccagctgctggggaggctgaggcaggagaatggcctgaacccgggagtcggagtttgcagtgagccgacatcgtgccactgcactccagcctgggtgacagagcaagactccgtctcaaaataaataaataaataaataaataagtaaatattggcttcttcaactggtgagatgaaacctatacaatagtcatgtgaatagcactaaacagctgacatggtgtaactcctctcagactgaggcttatctggggagtacaaagcatgtcaagaaaatgtgccttcatttccttagatgagtgtccccatcctccactctcctccactgttctcctctctgcttctatgatatcaacttttctttttctttagattccacatgagtgagatcatgtggttgtttgcctttctgtttctggcttatttaactgaacaagaaagtttttgacatgaaattaaacttctgcttgtaaactcaattcaaactatttacactgtcttctcaaaaatgttaacttattttaataaatctactgaatgaccgtatctcattttgttttatgaaaagaaattgtaagggtgctcaatagcctcttcattttcatactgtctagctcctgtgctcctattaaaattactgcaaatttagctttttaagaaccctttgtttcactacctgaagttctataaaaagatccaagttccttcacaaccgtttcttatgctgttattcgtacatatgtgataataccacgtctgaacacgtagataataagtaggggctgggtgcggtggatcatgcctataatcctagcactttgggaggctaaggcgggtggatcacctgaggttaggagttcgagaccggcctggccaacatgatgaaaccctgtttctactaaaaatacaaataataataataataataattagccaggtgtggttgtgggcacctgtaatcccagctactcgggagactgaggcaggagaatagcttgaactcaggaggcggaggttgctgtgagctgagattgtgccattgcattccagcctgaacaacaagaatgaaactccatctcaaataaataaataaatagaagtatgtattgtgttgcttagaaggtgtggtggaaattaacttgctgagtgagatcaaaggattggcactgaattgaaataaagaaatattcatgctgagtctggttcaaatataactgcacctgtaagaattgctttctgtaaactttccatagtataaaccaaatccaaatcactcatggctttacattcctgatcgttaaacttgaagcactttttaatactgcatgactttagccaaaatatcttagccaagattcaatgtttggttgaaccacactcacttggacatcttggtggcttttgtttcttctgaccactcagttatctatggcatgtgtagatacaggtgtatggaagccgatggctagtggaagtggaatgattttaagtcactgttattctaccaccctttaatctgttgttgctctttatttgtaccagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggtatggctgtgtacgttttgtgttacatttataagctggtgagattacggttcattttcatgtgaggcctggaggcaggagcaagatacttactgtggggaacggctacctgaccctccccttgtgaaaaagtgctacctttatattggtcttgcttgtttcaggcattaacccagataaatgccatgcaaattttataattattatgattgtttcaatttctggaagaaagttaatgaaacaaaaaatgtagtaaaatgccaaaggaacagtgacatttcagaaagaatgagggctttcatgttaattgtaagtcttggaatttctcttccttggagtaacaaatccctttgtgcctaatttcctaatttccaaaataaagttcttttacttatttctttatagtgacatcatctcttattaaatggcatatctgcatattacataacagttcattgccaaatacatatttgtgggaaatgagagacttaaaatacataccaaccagagatatagttttgaggtagattttaaaattctgagaagaattttgactgaatttttttgacaaacatgggacacgaataagattataccaaagatattataactttcattttaaatatggaactaatacagtatgaggtgtcaacaacgttgaagtttcacaaacatcaccactacaacagcaaaataatttttgctttttccctgccacaatgacctccttgctatttcttgaataaatcaagcatacccttgccctgacacgttcttggggaggcctgccctaatctatataaaattggagccattcttctcacctctggtattcccagtctccctactttttttccttctttctttctttttctttttctttctttctttccttctttctctctttcctttctttcttttcccttccttccttcctttctcccttccttccttcctccctctctccctcccttccttcctccctttctttctttctcttttttctttcttgcttccttccttccttctttccttttctttctttttcctttctttgccaaagtgttattcacctttaaatataatacataatgtgcttactttaatgtatgatttttattttatttctcccttctagaatgtaggcaccatgagagtgaaatatatttattttgttcattgatatttcacaagtgtctgggagagtttccaacttacagtagacaattaacaaacatttattaaattaaggagggaaggaagtgagtaagcacaacaactttcatttctgggtcttttataatcatatgcttagtataagaacagtgctattcagctatccaaaagttacaatcaaaatgattttggatgaatatcttgaaaattgtgagaaagaagttttatttgctggcaaactattctgggttgtttccacttcatgtaatcctaagtagcagccttaccttgatagcccattaaaactctgataataaaaaggcagaacaaaaatatctgtgatatatttagatttactacatgtacttacatgtctagtgtctggtgcaatggatgctaatgatggcaaatccttactgggcttctagtgaagttcttcagctaatgtttgaatgcatggttggtcatggtggtacccctttgtacaaaatatgcttttcaaataatcttattagggataataattatattaattcctggtttccatctaaaattttaattctatttatagcttcgtaagatttcacaagttaagagggacctcagattaaattagtacacaggcaattaatcagttttgtgtctccgacccttttcacgggctaatagaagctatagaccctcttagcttcagaaaaatgcgcactcacatacgcacatcaaagagcttaatgggaagtccattgacagaccctctgttcagatcaatcttctgattgtagagatgaggaaacagaaatctacagaggaagtgggtagtccaagattgcacagtcatttggaatagactggacaccagtagtacttttccagccactatatcacttccccaagcacttcctcaaaacttaccttcctttgggtctttatacattcagttatggacaactagatttaactagaggattttattgcttcagaatattaagcaacagggaaacatgtaccgtcttttattcacctgcatttaaggcatacaatataaattgcaaatggagcatgaaagtgcttaatcttttacaaaactgggtttgctttccacccatctaaaaatacttctatttattttaatatttaaagcagaaatctaagtgatgtgacaaaattaatcatttggagatatttcccttataggtagtatagtttcttactgatttctaatatgaaaatgaagccatagaacctagaaattgcagcatagttgtggaaataaacattggactgagagtgaaaatggctagtcttcctctctgctcatacaccacctgactggataacctttcgcagatctcctaaaagtctttctcataaaatgaggaagctctactagaaaattgttgaagtctaatttagcaataaagttctgagtttctataataattcaaagaatactctaataaatgtctgcaattgtggtcacatctatgggatgctaaaaaatctggatggtttcaatgaaagtatttaatttgttcattatgaactttgaaataatttatttcattttttaaactttgatcaaaatgaccctggtaaatagaaataagcaaactctttttgcttgaaatgcttattaatgactgcattgagacactcattcatcattcaagaaagaatgtttgctcacactgtgccagaaacttggaggaagagggatgtgacaagtaggggtactggatgtctagcttgtagaagtggattaatggctctgcttttaagatcaggaacactgaaagggagtaatggcaccggttttcacctttcatgccctttgagggtatctggtccatcaccctctagttgatgagggagggaaagttccctctcccttcacaaataggtggaaattaaatgacataattctgaacaaccaataaatcgagagtaaatcaaagcagatacctgttttgttaatttgatcatatgaatgtagctgcccttagtaataatttctaagtataagactagttaaaggacaaatgagttatcttgaattataagattttgttttacagaacaatattaactcttgtgtttagtacattagaataatagatcttttgatccatatttttactcatgtgcacataagaagttatcagtcatacaattcatttcttgaagttcatacctttcattggcagagtagaaacaggttaaaagtgcacaggcagaaattttaagtgcaaagcaacagtgatgttatatagagaaaatttatatttcctacttctattgaagaagaaagatctgcttgttctaagaatattgtacaaagaaagtgacttgaatcagcgttattctgtaatgctactatgcgtgcagtgtggagtagccactagaacacttggtctatcccagctcctcaacagtgtcttgcttgtggctggtgctcaaataaatccttgctgaactaatgagcatctctttcatgccacatggaatgctctaaaagagttggatcctgaagtttttatatttttgtaattttctggagttttagagagcaaaagtcctgaataaactgtgaagccactgcctgacaaataatacagcagtcagcttcgttatcatatcccattgagacacgacttatctacatgatgattaatagttttcacgcaagaaataagcttgaaatgtctgttgccttggatacttaaaacatccaggttcagcgatgttatttattgttgttcaaaatcagaatgaagttcctaagcaatgccattttggaaaaattacatcaatatattatgaacaactttttttaaatcttgatttcaaatggattgacacgtgtatattctgtaataatcctgacttaattcataaaaggatagctagccagttgtgtgctagatgaataaaaaaaaagcaggttttaaaatgtcaggtttgacattgtgaatataatatctaagtatccttttactcatttcctttgacttactatggctgtcatgttgggcttcatgaaaatttatttttaaacacttgagtgttatggaccctctgattaaatgattaatcagatgatgtatgttgccatcagctgaatcatttaatgttgatttcacaaacaagcacaggtcacaggcaacatttcagatttctttgaagaagcacacacaggtcacaggcataatcttaaaataattttataacaaggtagtaataagagatgtcaggactggagaaatattttaatttatagtaagctttccccttaagtgtctaataattgttaatataatacattgcctcaaataattaaaagtttggttcttgtccttgtgcttgacttcagaagataaccagatgactattaggtatatttagacctaaattaaaagctttgagacacaatgaattgcctgatttgtatttgtgtttcgagtggcatatactattactggcactataatcttagattaaagcatactgtgattattaaagaaaaatttaagattgatttgtttctaaaggtatgtaacagtgacattttgcaatgtggtatgtaaaagttggtatttctcactcatatgagagcccactaatggtacataaactgtccccacttagaaacacaattattatggcctttctttgtatctgacaaaatttcactgggttcaagatggatgaatagtgaattctaatgacccttaatcctgtaaggttctaggtgggaaagtactctgtaattatgtataaaattataaggaaaataggcttactgctatgttttcattaaaaatcattaactgagtacttaatatgtgccagacactcagctgggcaccatgagaaatacaaaactgagtaacatatgggtggctcctgccttcaagaaatgggcagttcaggccgggagactgacatatttaccctgggaaaaagggagcagctgtggtctctgagaacaatatggtttgttacaagtatatatccatcatggaaaaaaagagatttatcttagaaatgagagaggctgatgctctcaataaatatcatacattaaattgtgtttttgtcagtagactgaaattacctcacatacacgcacagatagtagccatgatattttagctgcttagatatagagacaaatacttccacccaaatcttaggatcagtggttaatagtctgtaagcattacaatcccacaacatatgcatgactatacatccaattttaatattcaaagaactgattgcgatgatagttttgtttgtcaaagaaatgtattataggatgagtgggatagaactgcatcacgttacaccaacaaataggtttaaatcatatttgtgcacttcccttgttccttcataaatgtttaacatagcttaaaattctgtggactgcaacgtgagagcaatgaccacacttctgtgaacccatttttactgtgcatgtgctaacgtctattgttagtattccttcacttgcaaagatggcatgataattttgctggtttcattaatgagatactgttaaatgtaggatgacttcaaacttagttgtattgtaaaattatttttaattgtatacatttaagttgtacagcatgatgttttgagatacttatctttatttatatatatatataatatacacacgtatataaaagtgattcctacattgaagcaaattaacatacccatcatcatatggttatctttgcttttttactatcagtgcctaaaatctactttcttgaaaaattaccagtatgcactacaatattattaacaataatcttcatgttgtacattagatctttagacttactcatcttacatgacttaggtttgtttttacctctactaccatctgagccatatttccactttgtaatttgataataaacttggaaaaatagcacttatatgtttaggtgacgggcataaataggataagatgtgtttatatattattccatatatcttgtctccaactacaatgataaacaacctgtttgtccctaaaaagtaagaaataacttgacttttctgccccttcaagcataggctgttagcttttaagttttagggagacattgatgatgctatttgctttatcaagaggaaattgtcaaaagaggtcttttggttctcaaactattcaaagtatttaaaaatcaggacaaaatatgtttacgtgatattcaagggtacagaaatgaggtaaatgagatgccaattgtatttgtcatgcaaatatataattacgtgtatgagagttagatgatacatctcatcaatttaattgttcttctacaaggagaaaatgaacaatttgtcaactcgtatatgaagtaatttttataagaaattttattaaaacttttaacaacatttggatttttaagttgcaatttaaatatccccttctaccaggtgattctggaatcactaagcagttacttgtgaaaattccaaagtagcatttaattcttattaatgtcatagtgaatactaatgcaaagaatactgagccagaaattatgcttgttgaataaatagattatttattgaacaagtaagtgaaaaaatggaaataaagaacggatatatattttatcttcctgcttagatgtgggactgtcctacttttctctggtgttcacaacaacaatatgataaatctaattggaattcagttcataggaatgaattcagttacattatggattgtgatgaataatgtacacttttaatttaatgaaatcaaatagattttaactatctatgcttacaatggggtgacataagtctgacaatccttaatatcaagtcatctccaattcacatgtatacacactttttttctatttggctattgggaatcctcacaaaaatcgaaaattgccctttcagtgtacgttacggtatttcatgccacacagattttctgaggttgtacatacagctttgccttgaggttccaatttttgctcagtggattgagtatatattatttgctatatatcagaagaggcatgtgcttcctacttatgtcaggtaactttgggattaatataattgtcctacaaagcatagatagatagaaatacttcatccttaatttctaatattatgacatatctaaagtaggcacctttaaaagttaatctccactaaatactaatgactgcttatagtggcaattcatctttcatggtagtcctcctacaaaggtatactaacatttatgagtttgaaacaaaggcaattcacaagtgttctgctagagatggtctatatctgctgtttgatccagcatgatggccagctggccctcctgtgcatgacggctcgtggtttaactgcaccattttgtttggtcatatacagggaaaacatggcatggtgtggagggcatgggcttgaattcagggaacagagagttggtcttctctctctcactctactggatgatgtcatctcccctctctaagcatgagttttcttatctgtgaaataaaaatgttgaattaaatgagttcaaaatgctttcagtctgtgtttaatagcttgaatcttaagacaatgtattcaattatgcgttgccagatccctggcaactcatgtaacctttctaaaccatagctactcatctgtaactggccagccaactgcccagggttggagtgtgaatgaaataagataatgcagacaaaagatttttaaaaattgtagtgcattatacagttgtaatattttgccaagaacttacattttctctaagaagtgtgtcgatacatgatcacagaaaatcttttccatattcctttgtagtttgatgatattaagtaagtaaattgtataacacaaagagggaaaagcatcactgaacatgccgttttatttagctaaataaaatgtaatcactattagttttcctctgatttccccaaagtcatgtgattccattgagtattatgcacatggtataattagaatggattctctgctcaaataattttgggaaacatttaaattaacaaagtttaaaagtatctctgttaagctgaagcaaatctcaaaggccttaatattgtatgtaagaggaatagttaccatctttcctaatgcctctttgacgccaaacccatggagaatagttctaggtgttcagtaaaacacagatttgggatgccacaggttaattggaactgtcccctgcaatccttttctctttttcttaataatggctgattgcaggtcctagatgaaagacatttagagagattatcaggactcagcatcccatatcagaatccattcttttatagtcattttctgttacatttcttgggacaacaccaaagaaatgaccatcttcattcacataggctttgtaccaaatgctgacaaagatccttggtgacctagatgggggcaggtctaagtagattgcagctgtaaaattggctgatgaatgatctcagccccttttactcacactcaaaggcaggacagtccattaaggggaaggagggcagagtttttccttaggccaattccctatgccagaactttttagaatggaagcatttccagaggagaaacaaccccaagcacagttcaaagccccctcctcccaagttcatttgaaagtgggatggtttatctgcaaagggggaaaagatgagggatagggacgggaatatccctacccttcagagagtctggtttcatcctgcacttttactgcacagccacaaatgccttggggtgaatctacaatatgatacatcatatggtctaaacgtgcctggctgatcctctctaatacttcaggggtctaaaagggataacatgctctcctgttactcaccgactctgtccgccatatttcacccagccagccactgccttcacttccgtccgaggcctaatctgagcccatgggaaacctaagaacccctaccacaactgcctcaactcttgggaatcagggtgtatgggggtgacaggaagtgagcatacattctccaacttgatatgtcagcccccacgtctgtatgaatgtttgctcacactgtgactgccggccttgctcctcaggctgcatcctaccagggagtaagacccaagtccttcctgctttcagacaacaccaagcctcatgagtccccactcagaggaaggaccagagacaaactctaatgttccactaatacttcccttcttattactttccttgaaaatcccttctccctctttctttttatacttcgctaatgaaaggtaatgaaagggtctggcacttggaatttagaattgatacatggtttttaacccgcggacgtattccacaataacccttgcatcttctactaagatgtgggctaggaagggaccagccagttcccagggtcacagtgcctcagctgatgtttcatattttcagcaactttatgttagagatgtccatcaatcagaacaatatggttagagaataaactaataaaagtcatttttgaggacatgttggaagtctatcaaaagcattgaaattatgcatgctctgaccagtcgcatgtctaagaatttaaatatgatcataagtttaaatatgaagatgtttatcacagaattgattataaaacaaaattgaaaaaaatagtgctagaagtttgatcatagggacctcattaaatgcattatggttgatccatgcagtggtttgctgaacagccattaaaatgttgtagaataattattaatggtgtggaaggatgctattgttgcagtatgtgaaaagaacaaattacaaagcagtttgtgcagcataatatttttattttttaaaaacctgtatgtggcttatgtacatataaagacgtggaataaatgcacaaggtactcagtttttctcagtgaagcccattttgcattttgggctgggtaattcttcgctgtggagaactctcattcattgtaggatgtttacaagccctgggccttacctctttaacgccagtaggcacccccagcatggcaacaagcacaaaatggtctctctcatattgcccttgaggaaattttgcaactaagtaactattactgggtcctagattacagtctggattattgcgttcctttcttatttttattttctccaattccctttaataagcatgtactggattcataaaaaaacaacataaatggtaattacaatattccgcactggttaaaacttatgtaaataagcattctgctgctttagccacaattgcaatttatgctccttctctttcttaagttcccagttcccacgtacattcattcgactgattcaaaagtcattttagcttgatagactcttaaaagttagagttatcatttctgctatttattctttcaattatccatttgtccacccatccatctgatccattttgttgatgcatgctgtgtataaaatactacaccagcctggtgcggtggctcacgcctgtaattccaggactttgggaggccaaggcgggtggatcacctgaagtcaggtgtttgagaccagcctggccaacgtggaaaaaccctgtctctactaaaaatacaaaaattagccaggcatggtggcagacgactctaatcccagctacttaggaggctgaaccaggagaatcgctcgaacccaggagatggagtttgcagtgagctgagatcatgccaatacactccagcctgggtgacagagcaagactccgtctcaaaaacaaacaaaaaaaatacaatgccaagcatcataaaaaatatagtgatatataagacctatttgttgtgctctaggcattgacatctagctgtcaaccattaatatgtgtaggagtctatctatcaatattatggactgtgcttgaagacttcttccccaatctttttctcttcccattaagtttgaagtgaggttttctgagtgaagtatcatagtacatacagtctcattatttttcaaaaatctctggttatagtacatttctttcctttatcccctttgttcccaactatcaaaccattttggatatccagtattggtatccagtattattaaaaagcaaaacagagaactattaacaaaaaaatttgtaggagtaattggttgtatggtatccagtactattagatagtaaatcagaaaattattaacaaaaattttagacgaataatggattgtcttgcccaagtgaattgagtgatttagttgttctttcatttttagcaagtacagctgatcatttgaggccttactcattgtttgattttgcaaattcttactattataaatgttttgggctctgagaaagctgttgtcttaatctgtttgtgctgttataacaaaatacatgagactgggtaatttacaaacaacagaaatttatttctcatagctctggaggctgggaactccaagatcaaggcatttgtcttcaggttcagtatctggcgagggccggttctctactcccaagatggtgtcttgtcactgtatcctccagagggccaaatgctgtgttctcacatggtagagagatagaaagggccaactcactccctcaaggcctttcataatgttaccaattccacttgtcagggctctgcccccgtgactttattacctctgcaaggccccaccacttaatactatcacgttggttattacgatttatcacatgaatttcgaccatactagttgccatcctttcattttcatatatccttaaaactttgcctttctcattttaatgtactttatccacagtatgccaacttttcgatacttttgttaacctgtctgacgatatataggaaactgtaaaagtgcagtttttgatacactctttagctgcccgtttacttctactgtcgttagagaaccccatccatagtgcatgtgtttattttgtgtatgaacaaagactttatatatagtttgggtcatttttattcattagtgcttcccttataatctctgaataccattttattagtacatactgctattcttaatagtaactagcatgcctgatcatcccaaatgtctaggttcacattttaaaataagttatatctttgggcttaacagtttattgaaaggtaacaaggattgagtcatagttgtatgtttttggaagtagaattcaactgtaaatagaaattggttgtttagatctcactatatatgaaaaaatgaaggctttaggagaaaatctccccaaagtacccatttttcatgtgataaatatcatgaaatgatttgagaaaaaaatgtatatttgttacagctaacaaatatttgtgttttttattcttcatggagagaatgaaatttcttctcttctttacacatttctttttcttattagaaactaattggtgcctttataaaaattaactgcagagcactaacgtgtatatataagtattatgtagggtgtagggtatgttcagggtatggtgtgtgtgtgtgtgtgtgtgtgtgtgtgtgtgtgtagctgtgtgtgtatataatgaaatatatggtagtgttgtttcagaaatctgcttggtcttcccagagttcattcatcttataaattcatctacattgatctctatttttggaatccatgaaatgttttttggcagtacttcctttaatatagtgtgctggaaatctggaaatttctagccagattagttacaaaaaattagccagtggttttgcactctctatagaatcaaggcccaaggcctactcttgttactcagggccttgttttatctggcctctttcttttcagccatatagctctcaaatactcaacaaaattcttcattctaggtagacaagtatcttcaaaatacttcccaattatctaataactgtcttaccactaagaaggcttttatgtctcctgtctgaattttatccatgcaaaaaagtccagcccaagcctccagaactccaaaaagttatccctaactgctgaaacacagtaatttcactatgtgaaatttcactttggtctcctagcatttgcagatataccatacatatccttgatccttttcctttcataccttttatatctaacccttaagctaataattttacctacactgtaattcaaaatgtatccccagtcttaccatgtctcccttctctactgttaccaccctaggctaggccttcatcatttctcacctggactccttccctaacctctgaactgatctgcctgcttccacttagacacccaacctagtccattcttgagcagtcggaataattcttttaagaaagaaaccagatcacatccccctctgctcccaaccatccagtgacctcttatcatacatagaatgaaatgcaaatctttactgtgttttaaaggccctacattatctggacctcagtaacttcttacttcctatcccttttctccttgtatgccaccctccaactacactctaactacactgtctttttccctgttcttcagacctgccaaccatattttcactgctcaattaatatgtagaaaatgaattgtttgttaaatgtagactgtttccttcttaaagcaaagataaatgacattgtcttcaaaaacaactaactgcccagaattcctgattttaattttaaaaagacaaactgcaagaatgtgttaaacagtaaggaaacaattcactacttcagaattctatatgatttcactgcacgttagtaattttgtatattatagaatatgagggtattctaataaacttaactctatgctgtatacttatcatgatagctcattttcttatatgtttataacagcactacttattgtacatggatacgtgggaaataaattaattttctccttaagaacaaagcaaccatttcactcatgagataaatcttgaagatttaaaaactacttataattaattatacattattcatataatgttaagtattttcttagtaaaccacataatttagaatggcaattggacagatgggcagaaccacatgcatccactattaggcagttggtgagcataagatgccagaaagaagattaggaatatcaaggcagggagcttccgatcgctcttgaaaacattgacccttcactcctcactctccacgatgcatttcctttgaaaagtaatgccttccaaaacaaagttctctgttttatatctaaacttactcaatagtttctcatggttattgatatataaaaaataaagtaaaatgtttaggcagaccaaaagaagaatttccccctccctctgccttttatgccaaggtgacagctatgaaatgtacagtacgtttcctctgcaaggaatgtagcagtgttccattgcaagaagatgagagggagagaaaggttgcacgctgaggaatatagtgtcatttgtcactgcctagactcatcagctgtgtggaactctgagaggcaccaggcttctttatttatttcttcagaaacttcagcaaaaaagatttcattaggagcagagaaaaatgtgaaaaacgaattagcttttgtgatggggagtagtcatctctgaatattgatcaagattaagagggttgtcttcgtaacttcttttatccatagtctatactgatttaactagaaaactaatttcaggtggtatttcgggtgtggcagatctttatagtaaatgaagaatctagtcaaatctactgaaaaactctgcttactttaatgtttgatctggttgaaaccattttagcttaacaatccttcctctgaaacagggaatcaattgatatcctacagcaaaattatgtggaagggccattagcttcacatccaatgcaaattttgcctgtgtttactcttccccaatccaaaatatatcagatcctagatgccagtgaaatcgtttgagctagatggcttgagggtcatagcttttttcatttcctgttctcagacctcttataattgatagaataaaatcagaagagccctagagctgtcccacctattctgcctcacaaaagtagaagtaatggcaaccactatcatagggatcatgctcacctttttcttaccagacaaatttggatattagcttgaaattaataccttccttaaaatgttggaatttggttatatgcgaaattttgctctatttattcattatattttgtatggaattatttttgccctatattttcacttaagtgttctctacccaagattttaattgaacccaaatcagccagacacacagacatggattttgctgccaccaaggttaattcttcttttaaagttaacttttaaaatttggtaaaatatagctttgaaaatttgcattcgtctagtgtttgttatgtatttcccccttttgtttgattatatgtctatatttttcttgtagaaattgatttttaacctgctttttatgttagcttttatgagcttctgtctgaattctgaatatgtctttcttaatgtcttctaaatgtttctttctggattattaaaagatttattaggcttttaataattatatttgttaccttagggaatgtgtttgaaaatattttaaatggaattgccagttaacacagcattgaactttttcttgttagagatacattgttttctaggcattttattgggagagaagttagtatgatataatgtctttggctgatattaactcttctaagatgcattgtttctgagaacaccattgtctgatttcattcagggaaatttcacacaagccagtagagtcaatacttttttcaagacctgttaattgatatatataaaaacttgccattgtttacatgcccatttcagatcctttatgtgacctaagctagaaatgcattttaacagcatttgtttttccaaaaatatttatttatttatttattatagagatagcgtctctctatgttgcccaggctggcctcgaactcctgggctcaagcaattctcctgcctcggcctcccaacagtgctgggatacaggtgtgagccattgtgccaggcccttgtttttattttttttgaacattgtattttgaaaggggtttgaaggtgatccctagatagcaaccagtaatgattcgagcagcaaaacaatctaaaaagtaattttataagaaaatgcagaacataaatgagcccataaaaaattatattaggttctatttacattactaccttctttcacatgtaatatttcactaacatttaatgaatttctgtgcagtgccatataccattatgaattctaggatagaagaatgagtgagaaatgttcttaggccttaggaagaaggaacaagcatctctgtgtaatagttatttcaactcttcttttacacctcattcccatattaaatctcagaaaagctaaagtaatagctatcccagatctattttagactccagacacttacttcaatgtcttgttctccttatcagactggaatcattccaaacctcttaacttctgggcaaccatgataatgcgacagaaaggacactaaatctgtcgcaaatttatcttgatattctatccagtcttacttggtactgaaggtcacaagtaaaataaggtggttgttttttgtttgtttttttttttttgacagaagagaaaagaacactgtgagcacagagtgaatgtctaacattgattcttgagtagcaggaattctctatgcgagaggatctctatgcaaaaagatctcatattctagcacaatttaaggatctctatgcaaagatatcccatattttagcattatcaataagctatggggtaatatattgtatgtggtgtggcttgaattctagaaatttgatttctagaaatggtccctgtagttaaggatatataatgtggccgtctccagttttctatgaggaataggaaaatactatcattattagctgtgtgaccatggacaacttgcttcgttcttcagttgcatcatctgtataaaataagaataagaaaatttacatctgcaaggtgtgatggagatcacatgggataattgtggtcccagagcctggcacaaaagggcttaatatttataatcctccccatttctccgtatactctaaaggaagtttattgcttatcaaattgtgccgtggttagttgtacagcttccctgccaaattgtaaactccaacactaatgtgacgttacattttatatagtgctatgattttcaaattgtttgcataatttcaaatacacagtaaattgctttttattagtataattattgctattgtcaatattattattacaacagcttcacagtaagatgggcagaaaaaaatttaatttccattttacaaatgcacttttgaggctcacagaagtcaaatagaccaaagtcacagggctagtgagggacccagaagaaacaaattgtaattcactgattccaagttcagtggttgccttactgcatcataaaggctattacacaatccaggtgtatcatatgattcttgtctatatattcatacatatcagaaaaagtgttctactcaaaattgctagcaatcaacagatactgatagtcattagtacttaaatctttatcaaatgaaatattaatacccatgaaagagaggacaatgaaaggtttgtatcatttgtatgtcacaagtcaacttttttcaatcactcattattagtttaactgtaaaaaattatttacatttagcgtgaaactttcctgtattctcaacatatttccttcggtagaaaagcaaacctccagttctctgttctttgcttggatacttgccagtttgtaactcagctatcaaacagtaaagctcacaaaacacttattaaaatgactaaaatccaaaacaccaagagcacagcatgctggtgagatgtggagcaacaagaactttcattcattcactaatgctggcaatacaaaatggtacagtaactttggaagataggttgacaatttcttacgaagctaaactatacttaacatatatatttgtccattttcacagtgctaaaaagaagttcccgagactgggaaatttataaaggaaagaggtttatttaattgactcacagctcagcatggctgaggaggcctcagaaagcttataatcatggtggaaggagaaggggaagcaaggcacctacttcacaaggtgacaggaaggagaatgaatgcaggaggaactaccaaacacataaaaccattagctctcgtgagaactcactcgctatcatgagaacagcatgggggaaacagctctcatgatctagttacctccacctggtctctcccttgacatgtggggattatggggattataattcaagatgagatttgggtggggacacaaagcctaaccatatcaccatatgatccaaaatcatgctacatgatattcacccaaaggaaatgtaaactgtgtccacaccaaaacctgcacatgcacgtttatagcagctttattcataattgccaaaacttggaagcaaccaagatgttcctcaataggtgaatgaacaaaaagactggcacatgtactcaatggaatattattcagtgataaaaagaaatgagctatcaagccacaaaaacacatggagaaaacttaggtacgtaagccagtttgaaaggttgcattctatatgattccaatatatgacattctgaaagagacaaaattctggagacagtaaaaagatcagtgattgcctggggctctgagaaagtgcagagggatgaatgggtgaagcacatggcatgtttaggacagtgaaactattctctatgatactgtcatggtggatacatgaccttatacctttgttaaaactcagaattttacaatacagagtgaattctaatataaactatggactttagttgtaataaggtatcaatgttatttcataagttttaataatgtaccacactaatgcaaaattataataataggggaattgggggaagggtaatggagtatatgggaatgcactgtaatctcagtacaattattccacaaacctaaaacttctttcaaaaatacaagctattggtcaggtgtgatggcttataccagtaatctcagcactttgggaagtcaagaccctcagatcacttgaggccaggagttcgagaccagcctggccaacatggtgaaatcctgtctctactaaaaatacaaaaaaaaaaaaagaaagaaagaaaagaaagaaagaacagaagaaataaaagaaagaaaggaaagaaagaaagaagaaaagaaagaaagagaaagagagaaagaaagaaggaaagaaagaaacagaaagagagaaagaaagaaagaaaaagaaagaaagaaagaaagaaagaaaagaaagacagatgcggttgctcatgcttgtaatcacaactactcgggagactgaggcatgagaatcgcctgaactcagaaggtggaggttgcagtagggtgagattacgccactgcactccagcctgggtgacagagcaaggctctgtctcaaaaaaaaaaaaaaaaagctattaaaaatatgtaaagctcagtctagatacagtaccagaatagtaggaactttatttcacctgtcctacaaattatggttgtgtgccacttgggtaaaactcagaatccaaatatgtgaatgtaagatttatggggaaattatttgtatttcaaaataatccttaatgaatgcactccttctaaagtagccattaataaagcagttaatgtttcatttaattatagattaatgtacataagatatgccaggaatgcaattaggaactgggaagggggtgttattctaataacttccacatagcattgtgagacattttctgctttcttcaaatttcatttaattacattttaaacaaatatttttgtgagcctattatatagtccttcgctagcactgaggagacatgctttgtgaccttggtgatttcacattcaaatttccctttcacctacactcttccttgttttttcatgcctgtgtagattgtaaattcttcctcagattaagacattttattcacctttgtaacatccacagtatctagcacaatcagtgccttcaaaaacaattggcctcaagaattgattgactcaatgagtgactgaaagactaaattaataagtacacatctatttgtacttccctgcttacttataaggtatgacaatgaaatactgagacagttatacattacttacggactcaatctcatttctttacaatctctattcttcttttttgagtataatgttattttacaattccactaacttgtcactctttattataaattcatatctccatttcacctgagaataataaaggcaaggaagtattttaaatgatcttgttttttataactagcattcattgagcaaatcaaagtatgaaaataatataggtgtcagtgattattataaagttgtatgcacaaaacattccaatgattggggccaatacagagaaaacatctcaatatttggaattttgcttttctgtaaatactttgatatgtacttacatcatatcaattataactcctgctgaaaacaaacagtgcacacaaatttggtagttggaggagactttataaagggactaattacgaaggtttagaccgggttaggaaaaacacacggaatagtgcaatactttaggatggcaacagcgagcaccgttataaccactaggccaaaatgaactaaatgaacagggagattaccatttatcagaaaaagagggagaaaggaaggagagatgaccaagcaagtcctatgtgaagacggctgcctgacttgagctgtgtgatctttggactgataccacctgcctgcactggcctagcagggcgagaatagtcaatatctggaaaatggatcacctgaccttactttcctccctccctgtttcctctttgtggtgtttccactggccaaactcacagcgtagacaaaaggagtgcattgatgtagcagtggttctaatccagggccaattgtgctcccagggaacattagtggttatcacagctcaggggaggaagggagaggagtggagtgctactatgattcactgagggatttttttaaacatctacaatgcacaggacatccttccacaacaaagtatccagttaaaaaatgtcattactgccaaggttgaaaaaccgtggtgtagtcagtacaattcatcttctccaggcacagtgcaggagtggggtggagtgtctgaaggggaagaaggaagaaaccagcacaccccacaaaagtaaccaatgcaaataccaaataggaaaagacagcacttaaaatacaaaagtctcaggaatatatctgatagtgttttatggaatttattaaaatttagcctggagtgagtaatatttagcaagccaggtttgtctttagagaaatccttgtggggtttatacaaggatttattaacaaagggcacacacaatactcatattacagtcagtctggttatgtaaaacatgggcaagaatgtaataggacaatgtgatgtattcacaaaggattttaggactacacagataatcctctaatgctttcacttacgtactatgaaaggctatagtttgcatagtgatatagccacgtaagatagtaaacttgacattcatgcagctatacatgtttgcacacaccaggatgcatgccctttctacctggttgattttttattcttttattaatctctaatttattccccagaacactctccataaaaactttctcacaacttaaatctttaatctattgtgtggatttctgactcattctccaagcttttcctcttccctccgcaatgccttatagtcttatgactatttatccctttgcctacatttctagccagatctcttgcctgatacacactctcatatttctctttgcacgctacacatttttatttagatatcacactactactttgatttcaacaggtctcagtttaacttaatttttccttcaagcaaggagtcccttcatatcagttatcaccattggcaccagaatttttcttatgacttcccatgacctacaatataaaccatataaatcactgatgcctccatagttccctccctctcaaatttagccataagatgattttaggatccttgttttttccaatctctctttcattctctcccccatctcttccattatgaaggtttggataggacacaactcatgcctagattagtgcaatagatgctgagcctgtgcagcggtagtttagctttctctcctggttaactttaactgccacatatatcacttcacacgtcatttttcattcaaacgtatttaactggctcttcattcataagaagctggaatttgtcgtttgactgatattttaaagattttatattttttctccatcctcgttctaatgttgtatcttgtgtcatttgttcattcataaacttaagacttagctaaccactgagcatccaggaaattcagtatctatcatgtgaattctctaatactggttgatccattgtcaccagagcatagcaggcttctcctgcctttatgtatgtttgtcatatagttcatgcctaaaattctttcttaaatcttaaattcctaagatacacacttttgcccaagatcacagtaatctctgccataatctctgctggaatctgttcactgtgttgctcctgctaaacttcttacagatgactttttttctttttggtttccctggtatctagtataatttcttatataggtactcaataaatgtttcctgttgatctctacacctactctgtacaataccatagtgactagacacatgttgctatcaagcatttcaaaagtagctagcctgagttgagatataggggtaaaatacacaacagatttcaagacatattatgaaaaaaacccataaaatttctcagtaatttttttatagattacatgtagaaactataacattttgaataagttgtatcaaataaaatataaaattcacccggttctttttaatttgttaaatgtggtggctagaaaatttaaaattacataattggctcacagaataattataatggatggtattgctttagatcaagtttgtctaacccgtggcccatgggccacaagcggcccaggatggttttgaatgagatccaacacaaatgtgtgaacttccttaaaacattatgaattttttgtttgttttgtttttgtttttttctcatcagctatcatgagtgttagtgtattttatgcatggctcaagacaattaattcttcttcaaatatggcccagggaagccaaaagactggacaaccctgctttagatagtaaagcatatgagtagttaatgtgtactataagcagtgtgatctgatagactatttaatgttgtttgatggtacattattcaagtcgattattatgtctacctatgcagtttaacgacggtaatgagagagggcagcttgattacaggtcttatcttttgactaacttgctaggccacctgagaaggacccaaattatctgaatgcttaactcaactaatttgtattcacttgaagaatttcaaggatgtttatatgccatcaacttgctttaaattttttctctcagtgaaaatttttcttaaaatgagtatgtggtattcaaatttatccttgttttctatgattatcttttcatagcactgtggtttccaggaacctttttttttttgagatgcattctacatgtaactattgcacagtttgcatgtagtaaggttcattattcttctacttttccaaacacctggcatgtttacttgaggttggtacaccttgtatcccagattttgctgtttttaacttaaatattgaatattttgattaaacattatggaaagtttaaatgggtcaagaaaaatagcttttcttcccatgaagaacaatacggcataggagttaagagcatagatttaaagtcagaaaacctgtgctgcctacttgtgcaaagtcacttacatgctgtacttctgtttcttcatctgtaagttctacccctaggtatttacttaagattaatggaagcatatgttcatacaatgacttgtacagaattattcacgatagcattactcttaatagctctaactggtaacaacacaataatcaatcaacaattgtgctgtattcatacagcagaatactacttagcaacaaaaatggaatggactactgataacctcaacaacatggatgaatctcaaaactatcatgctgtgtgatgccaggcacaaatcagtacatactataattccagaaaagacaaatgtcatccatggtaacaacaagatccatgcttgctggaggtagaggcatcagttcagtcattcaggaagctgattccaagatggtgttagaattacaaccatccacaagagatttattgcaggcaatagctatgaaaggtagaaagagaacaggagaaaaaccaggcaaggaaaaaccacaatgtagttgtgatatcacttcaaagggaggcagaaggaaggagaattgggtaggaatagccacagattacagtgcagttacaagaaagtcttggcttccaacaaaggttacttgttgaggagtcatgcattaggcagacatgtctgggctgtagtttccttgctgctcccagtcattggctggaggccagtctgggttcctgtgctgtggtggatcccattgctgctgcagcaggaggccaatagcactcctggcagctaattggagagaaaagatccaagaggtgtaccttcatggctacccccatggggctggggtggaggtggaggagaaggagaaggaattaactagaaaaaggcacaaaggaaaattggggaaaataatgaagatatatgatttctcaattgtggtggtcgttacatgggtttattaatgcatcaaaactcaagaaatgtacatttaaaatgagtgcatatgattgtaagtgaattatacctcaatatagttaattttttaaaaatcatagatttctttatatttaatgcatgaacataaacctaagacactcctccactccaaaacttaattaccttgtgatcagcagagcagaaggtactttgtgatatataggtagagaagatgaagtcttgtgacatttaacaagggacaggaaaatggaccttgtcctaagttaccaaactgcaaaaatatcacctacaaaggctattcataacatacattttcaagggggttacaatatttgcctactataaaattttggatctgtaaaggggttaaattatttgtgcaggggaataaacatcaaagaaacattaagaggtccagagaagtaaaataggaagggtcttttggctagaggagatatttaactttcagaacatgtggaattaagttgtattgattatgatctgatcttcttccccctaaatttgatcctcttcctgtaatctattgtttccatcatcttcaactcttccctttccctctcccttgtccctcagttctagtcaatcacaaagtcctacagtttcactttctgtataccttatttctggaattcatctctagacttcaaaatatatatatatatatttttttttttgagatggagtctcgctctgttgcccaggctggagtgccgtggtgcaatctcagctcacagcagcctctgccacccaggttcaagcgattctcctagttcagcctcctgagtagctgggattacaggcatctgccaccacgcctggttaatttttgtattttcagtagagatggggtttcgccatgttggccaggctgatctcgaactcctgacctcaggtgatccacccgcgtcagcctcccaaagtgctggaattacaggtgtgagccactgcttccagcccaaaatatcttaagtagataattgcacgactaatctctgcttttctctcccagcagccttccaaattcatgtctcacagctgacagagttgttcctgccttcagattcatgacctggctctgtgttctagctcaggctttctctctcatatcacctcttgcctctctgttgcccccatattttcccctctggttggttggtgctcctttggaaccctctgcatatcttttcaagaatattatgacttattatgcctataaactttgtttaattatttatttctaaaatttgacagggaactttccgaaggcaggtattgtgtctttctcatttaaaagcaaattctcgcctggcatggtggctcatgcctgtaatcccacactttgggaggctaaggtggacagatcacttgagcctaggagttcatgaccagcctgggcaacacagttagaccaaaaaaaaaatatatacgaaaattagcctggcatggtggcacacccccgtagtctcagctagtctggtagctgaggtgagaggatcacttgagcctggatggttgaggttgcagtgagctgtgattgtatcactgcactccagcctgggcaaaaaagtaagatcctgtctcaaaaaaaaaaaaaaaaaaaattagtgaatcctcagtgtttaaaaagtccataaacatactaaacatagaagacctccaaatgaaattaatcaattattatttagtgggttgcttctcttttgttttaatatagttttaacaaagagtaaaagttatgatctttttatatgtaaaataaataatgccgggtttgacataaattttaggaaaactagagacgctacttcctaaaaattttctttctataatcttcctaaatatttttccataaagtacaaaataatagaaaaaaattaagagattgagtatcctttcaggaagtgatatgacaaatagggttcgagaactatttgaattctcaccacttttcataagggcagatctcaagttaaatttttctattcgaatttaaatgactttcactggaataccattacagaaaagcttctgtgtttagatggcaatatggagtttcttttcttggaatattaattgaaggagaagtcttaattttttaagtctatatctccgtatatatttgaacctattttatatgttagtccttctctttagtaaccttcatccacagtgaacaagatttacccttacctttaagcagtagcggctactttatgtgaagtgaacagctgctttttttatctgcatctagacatcaagtagtccagagtcctttctaacaccctagcaatagaagtaagaatattttgaccattccatgacttgatgatacttctagtaataatactgtattattaaaaacaaacaaacctttgtgcagtggtaattgaagcagttccttgggaacatgtattaagtactttttagcagttaagtccactctctgtaggttaaggaatatttaaataaaataatgtggcaaatgagttcaagatgataaatgcgatgagaactaaaacagctttaattttatgtgggaaataaatagaggaaaagtacattacagggctcctggacttatttctttcttcaaagtgtttctcctagcgaatattattactattttttctcttaagtaaaaaatacacaaagtatgaatctacacaggataataatattgaagttaaggatgatgtctcctccttcactctccaaaatactatttacttggcttcatggaaatctctctcactccaattccaccgtgtcaactgaggtcttctgttctttctctccctatagcatattcctgttacataaatcctaaactgtgtcgtgttagtcacacactgtaacctctagataagcgcctgtccagaggttctcaatcagagccttgcaaatatgtattaaatcaatgggtcatcttcagtgtctcagtgggcccttggatatgttttgcagactgctgtgagtatgtagggatgtccagtatcgagggaagtgtggatggctttcattggttcttatagggctgaagaacacatagagcagtaagcacttctactgtagggagagatcgagcttctcccatccccactgctggcaccaccaccaccctacaccccattttgagttctgaaagtgaatccttgagaaagaacacacaaaacaaccatcataatagtgggcacagctgtgggtggtagaataacattcccaagcttcttttcctacacatgattaatattaattcagcaaacatttattcagctcctacttttaaacaggcactattctaggtactaaagacatagaggcaaagcatacaagactctgcctttgtgaaacaattaagaaataagtaaaaagaaaagaaacagaaaaggcaatttggatagtgtcaggtgctataaagaaaacaaaatgccattttaataaataataataatacaatgttttcatactatgtgctagacactatgctagtaggtatttatagacataacctcaattaatcctcaaaatggcatgttgatatcaataccccaagtttacatatgagacttaagatgtctgagtatattcccccaggtaacaattaatatgcacaataaaactttttgctcattcatttattaacctatgttgattgagtacctattttgtgtcaggcatcattttaaggcacctggatatagttatgaacaaacaaataaaaatctctgccctcaaataattaatatctcacagaggttaggcaaaatataatcagaaaataagtataacgtataggatgccagatcatgaaagaagctatgaatggcatcaagaagctggaaaaggcaaggagacagattttctcctagagtctccaaaacagaacacagtcctgccgacaccttaactttaggctagtgagacccctattggacttcagacttacaatcccacaatgtaataaatttgtggtaattcagtaggggaacaatagaaaactaatacgatatcaaaacaaattatatcatagaacaagaaaatgtaattgtgacaaataatacctacaaaaatgttgtaaatgctaggcaaataatgtgtttaaagcacttaggccaatgttcaacgtaaagtaattcatgctataatatcatcatcatcattaccaatatttaggggctctaacaaatgatgtacgtgtaagcagatgtaagaaaatttccttgctgaagaggaggtattaatagagtatataacaatagataacaaattccaaataaaggcaaactaaatgttttattggattaaatttaattttaaaaactacaagaggccgggcgcggtggctcacgcctataatcccagcactttggaaggctgaggtgggtggatcacgaggtcaggagatcgagaccatcctggccaacatggtgaaacgctgtctctactaaaaatacaaaaattagctgggcctggtggcgcgtgcctgtaatctcagctatttgggaggctgaggcaagagaatcacttgaacaaccaaggagtcggaggttgcagtgagccaagattgtgccactgcactccagcctggcaacagagtgagatcccgtctcaacaacaacaacaacaacaacaacaacaacaacaacaacaacaacaaaactgtgagatccatggtgggcttttaagaggaaaatgcaagctaaggtttgtttagactctgagtactgcatgtgtaaaaataaaggcatgatgaaaagatcaagagattagagtgatactttttatctactagtgtcagagtcatgaccaggggattggctatgagaatacataagctgtgccaggagtaatccaaggagattgtttcaatttggaagagtgtccacagaatgattctcatactagacgttgggctattgtaaagaaagttggtaggtactccatcgctaggatcatatcagggagaaattgaacaggatggccctaatgaccctgttgtacccctagcttatggattaggcaagtcacttctactcgtataccctgtttccccatttgtaaataagaggatgtgttactctaaggatctctaagattctttgcagttgttaaattgcatagctctccactgattccatggtggaaatttgctattctattacaaatattctaaatgtatgagatatcagacatactcatttaaaaaacaaaatacaaaaaataagtattctacaaataaacacagataatgtttaaattctatatgtctttgtttctcttcagaagcatccaaaatacaaaccatctaagaggcaagaaaatgtcgtgatgttcctagtgcaagttaaaaagatttgctttcctcaagtcggaaagcccttctcatttttgaggtttttttcttcttttttttttcaagtgaaagcattttggaggagtcaatatccatctttaaaggtagccaggtcacatgtatacatatgtaactaacctgcacaatgtgcacatgtaccctaaaacttaaagtataatttaaaaaaaaagaatttaaataaaaaaagaaaatcagagagaaaaaaaaaaagatgcatgtgcaccctgatactaccatccatagtgatacggtttggctttgtgtccccacccaaatctcatcttgaattgtaacccccatgtgttgagggagggaccttatgggaggtgattggatcatgggggtagtttctccatgctgttctcatgatagtgaatgagttctcataagatctaatggtttaaaatcatggcacttccttttgctctctctttctcctgccatgtgaggtgtgccttgcttccccttccccttctgctatgattgtaagtttcctgaggcctcctcagctatgcagaacggtgagtcaattaaacttctttctttataaaaaaaaaaaaaaaaaaaaaggtagccaggtaaaaattacttgtttccaggacattttcacctgaaagaagcattgtcatataacatagaagcaagaaatccagtagtgggggttatttaaaaatagctggaaaatttcaatcagcatgagtttgaagcaacaatttatcatcaccttttatggtgggtggggttaagaacatttcagcgggcaaagtggtggtgatggggaagagacaccaggggaggtgattcccattgcattgctttgtaaacagaggcacaggttcttcatttttgtcacacaaaatcacagctatgcagaatttattaatttattcttctgagacaagaaaaaagccaccaaaggaaaccaacagcttgctcctctcacactgggggaaccatatgagagacttatctatccctgactttaattttgacctgaggagagctcctcttaaggaaaacaaattaattcaatgactatactacttaatcattgacctttatttaataagagatttttccataggatatgctgagctgtctcacttacatcagttgtgtctcctgaggtgggtgacaggagaccacaaatattgcatagcacacaaatcgttaatagcagctgtataccaaaccattacctaaatatgtagagtacaattcattctcactaatgtcagagagcatgctataaaatggtgaatccggacagctgaagatactgaataataacctctattttgaacaagtttacagtgttccaatcagtaattaaattgatacctgatgaatatatgtgtgtgtatgtattcatagcagagatggttttcctgagataaggattttgttattcggataggctgctgctggaattgtccttctacccttgtttctttgtccttagtcatcactcatacctctttccactcttctgccatcacttttgtcaccaaagtcatggtcctttccccgccgattgctgctgcaggtctagggcaccaagacttaggcagcactcaccatgtgccaagaactggaccacaggtaccatccagcattgctcatggagactctgtccctttctgtaggacaccctccttttagctagcaacccctccaccacctagagcctctggacctctcattttaatattaagaactaggaaaacttaccgctgagaataactagtacaactagaactggtagagaaatctgggtctcttgggaatggatttttaggctttattgattagaggtgtattaataatgcagtgttatagtttcatgacataacgaataaaaaagttcattttggacttgcctttcagctccctaggagctaaaagacgtatttaatgtaacttgtgtggtggaaataagttcttttttcaggcaaaagatgtgcaaacccatctggggaagaaacattaaaaactaaggagacagtgtcctagataactatgttcttttcctgttttagtctaaaataatgattagttttcttatatatcttcatttgtcttggttccttttagcccaatttaataatattattgcagatattgatgaaaacctttaccttcctcttaattcatcaaagtacttgataaaatttatacatagtacattaattgggaggtttttatgagattaattaatataatgaactgatgttgaaattatttaaaacctgaattattattgtattaagtaggacacttaatacagttaatcagttctgtctttattcatttgtgagaatttttggcaagctattgtgaatattcagggaagggaatgtatttttagcaggaatcttatacctcctacatagaaatgaagcatttactgaaacatccatgaaacaaaatgtttctgaatgtgtactatacacttgttataagccccttttcttctgtagctatattttggagaaaaatctttgctttgacaaaaaaaattatgttgacttacacatatattttataactaagcagtgtttggtttgtgataaaggatacaaaaatataaaaatgttcagcacacgtaagtaaggccttgttgacagtgtgagttatgctactggatactcaaaaggaacattcagtgttctcaggtggtctctagactgtctcaagcctaggaagatattttataagcaaaggaataagagaaggaagattcagatttaatccaagtgaagaattcagttttgtgtgccttatcctgttattttgagaggcagccaaaagatgctggtcagcaaggagaattgtaagttgggcagccaactctgatttctcaacctcttagctgttttcttaaactcagaatttttaatgaatttaaatgtccatatcaggtagactttggggatgcttttaccagtgattttcagaatgttactttctggcatttcttttcacgtagcattatattaaaaatgaattcattcatccaccttcccttgtccttactaattttccctcctactcccttcccccttgttcttgccatggggacatgcaaacactggtggttgatgtctgagcaaggctgctgacagggggaggaaggagatgtcaagcagaggtcaatggcagtgtgcccagcagcctaggaagtaggagggaaaagagagagagacagagatggtggatgaaagagaaagccaggatgattatggtggttatgatacttgtcatgctgaacacccaattgagcacccaataagcacataataatttaatcatcctctggcttggatggcagtgttctatcagtgttgacttcctggttgtgacagttttacagtgttagtgtagaagagaatccttgctttagagaggtacttactgaagtacttagggttaatgcaccattgtgctggaaaaagatacgcacacacacgcacacacacacacacacacactctcacacacacgcacaaatacatccatgtgttaggcagagggagcaaatgaggtaaaatgttaacaattaggaattctgggtgaagtggatagagggactctttgactgttcttgaaacttctctatacatttgatctgtttcaaattcttcagaaaatcaaactacaaaaacttaattcatttagtgaacatctactgaacatctgtatattaaatagtgttaaatgaatgtcaattaaaatgctcaaacacagtagaggttgattctcattcacataagtccatggtaggtgtttttggcaggtgggtgagtttctcccttagggagattgaggaacccagactcctcccaagttgcagccccaccgtcttctgaggggatgcatccatacccacttcgaagtagcatacattatttcctttctcattcctttggataccagccacaatttattcaaggtagacagaaaattgtagtatatagccatatgccctgacaaagaagggagaacagattttggtggacaactagcaaactctgatacaatctgttattaagcactgtgtgtggatagatgctaactagaaggagattatcttcccttcagcaaatataaactgaatgccgtttatttggttgaaactaagctagatcatgggagtatagaaattttataagaagacatagtcacttctgtcagtgagctcaagaagaattagtatgcggaatgtaatcatacctacagggggcttgtgccacttaagtaaaatgaaacattattttgagtacaatttagcaataaatgtactacgagatcattaaaaatcatgtttgaatgttattgtgtcaaggatgggaaaaagacttttgggttgtagacttgataattatagttaaaaacagtttttattcttgtttagtcttattttttatgtttaaacatatttatacttgctaacatttatacttgctaagtaaagactgtttttacaaccatgacaagaacaaaacatattagtaatgcaaatgccacatttcctacaatcaactaatcacactaacatatttgcatggaagaatcactgggattgatctggccacgtgtgtagtcatgcccaaaatgtgaagtccatctgttttgcaattttttttaaccactgttatccaaatgctccttggattttttttattagtggatatattttggaggtcagacaccctcttggctagatcatcacctttataacaaatatatatactattctcatggaaatatatttagacattgccctactgggaatttttttcaagtaattaatgtacagcttgtgcaacagcttgatcttggcttcatggaaataattcactcttagcagcatctaatgccacaaagcatttatggatgtcagctcagaacttacttttatttatctctgagttacttttttttttttttttttgagacagagtctcactctgtctttggcttgtccctaacctcttaacagacttaatattaagctccatttcactcagtcgttctgttgtcatataaatgagacattctacaagcatagtttttagtttctgccagagcatcatacaacattgtgagctatgatgaagataaagacctagagaagatatttaatatgaagttcattatctaatatttggtatgtgtggcaaaatagcaatctactgcttggttctgctgtaatctatttacccacccatcccatctttctttcaatttaaaaggataatgattttagtcacgattatacataaacccattaccataggcaataaacaatggggcaaaccattggtcccatagttggagtgtggtctgaagtgtgttttggtggagagagatctatgtctggagatagctaacatggatttggatcccagatctgctcctacctgttgctgtgcctgtgaccaaatcatgtgatctctctggtttcagtttacttgtgaataaagtaaataccttcatcaacacctgtttttgaatacaatgtttttctgtaatttttgcttcttataatgttataatgatcatccttacatctaaatcttggtttacattttcatcaattcttttggaaagattggagaagtaaattttggagatgtatgtcggctattaaaaatgtttaattttttaattaaaaattaaaacgttgaaaaatcctgatgcaaaataaatgcattatgcttagtgaactcttctcatttcgaagtttattcaccttcttgtttttgcaagtttcctgaaaaatgcatataaagtcactaagttagcagaactttataaaattatataactatatataatcttttgatatcagtgaagccagctgatcctatagaaataatgtaggaattataatcactagcacataatttaagagtcctgtggtcttattcatgttatttaccctctctgaatcttacatatagtaagagggttattatacataatatgtgtacatgtatacaggtaagtaagtatatatgcttatgtgtaaaagcagagttattgtgagagtcaaatggaaatgtgaaagtactttgtagttttttattactattattaatttttaataaaatggtaacattcatttaataatcattagttttaacttcagattgtactggatttcctctagtatttcttaagattagtgaataaagtatttctcctaataaatatattgactactgtctttcgatcaaacatattaggtatatttttacagtagcatcaggcagtgaaaatttgaagctctttatagaggactgatttatgatgaaaaggaataacatgaacaaatggaattatatgaagcttccccagaaatatctaagaggggccaattttaagaaatatctgacttctttttcatggacatttcaaaataaacctaactcatatggtacagtttttaagagggaaaagaaaaaaccatctgagaatctctggaattctgccgaaagtatcacttggcattttattctaccttctggatgcagttgattgacagtagtgttatgatgccaggggtatagtgactagaaaaagaaaaccagggaattcagtgttcttgctcatgaagaacagcttggttctttaaaaacaatgagattttgccaccccatctcacaaacctatgatttgtgagaacaatcccttttgtgttgcaagacttttacatttctcttcccacactatattagaagaataaacattgcttcataagtaccgattgatagtctcatttcatatttttaaaatagagttactttaaggttaaatttttcatgtagattaaaatgactaagtaaccattcacatatttcaaataaaatatatttttactacaaaaggaaaataactagattcttaagtgttatagtcaagtgtaattgagtaatatgaattctaaatgaatttctaagatctgctcagctttcactactttaggaaggaacaacttaagaaaaattttaataaagatatctcttcacacacatggcagtgttgtacttagagaacatgacccaaaattttttatgactgcatattgaattcctgatactcttgggaagctccaaaagcaccagtggagtttccagatgtaactgtggctgcagacccgccagtcccggtgttggaagggatcattataggctcttgtgtgcagactcatcttcagacccagaggaattaaataacttgcccaaagtcgcacaactttctcatggtaggttgggcactagaataaatattgctttttcttaagagttttagcctccgtattatgaaatcttctatgttctgctgatgatatctcctttcttcatctgttttctatttttaagcaatggaaatacaaacttgcaactccccatttccaacacaacttagaaaaaacaatatttaaagaaaaaattacaggcatctcatctcctttacctgacagatgcttgatagtaatggcctctagatagggatgacatctaatataaatgtgtcctttcaagtcaagctttctctgttcattagtagaaatattgtatatcaagtgtgcaaaaattttcttcaacagggagctttgtttccctccttttattataacaatctgagctttgtggtcccagggtctcctagtgcctgtctttaggtctgtttattcacatgaagaaagcatgtcatatagtattatctaagactcaggctgcttatgcatgatgacagaagggttcccaggcacaaacattcatccatgcattcatccatccacctattcatccattgatttggctgataattattgactactgttgagttgccctcagatttagtttctgtccttctgccatggggaaatatggggttaagccacaacatactcttctcttctttttctgcaccttcttagtatatttagttccattttgtctagccctgcctctgacttctttgttgtacttcaggttttttatcattgaaagttatttctggatcatagatcattctcttggtcactttgcttgttcacttataaaattaattcagaaaaaatgacccacagtaattaccgtaaatcacagaccataaactataatactgtatattgtattatagtacagaaatatttatactttaaaatgttttaaatatagatattataaaaagatatgtctcatataagtaatataaatacttttttattacctcttctctccctattctccaggccagtgttttaaaaatccatctttatatgtccatcctggaaaaaactcatgatcataaatgagtttctcaatagagtttataagcccacagttgaaacacaattgtcttagcatccatttagttgtcatacttttaagatttaatggcaaatattatgttttgtttcttcaaaagaaatattttaaaattttagtaaaggcagttagagaaggtagagataatggactgtttaatcctacttttcatcccacaagtgaacaaaaaaatgataaaacatttttcccaaaatgtagctttaactatacttaaatttggactaaaatgggagatatcttttctactattgaaaagccgtgtctgtagattaatgctaaaatcgggtgtaaaagcaaaatttgtttggcttgattgccaatggcccattcatttggctacagaaacaatagcacatagcaacagataatgatgtgagatcacctagctcaagtaagagtgtctgatccgtcaaaaatatatacatcaagattcaaaagaaatgtgtgttttctcaagtcatctctgtaaaaatacattaaatagaggaatagaagtttgactttgaaaatacattgcagacccaatccgtctttcctattttctggtgaaaagtatcaaatatgtggaacctggaactgctattctccttcttaaaaatctttcttaatattctattgataactggtgcaagcctaactttttgtcttacccgattcttctcacaccaaagtgataggaccttcaggtagcctttggatagaagataaataataatttaactattgatggaagttagtattagaattagacttggaagtctatggaataaaatgattctacaacaatttgtacttcagacattagtataacaaaacatgtttgcccgtgcatgcggaaacaaccaatttcatgtggatgcttatattcacaaaggagtaaccacctggggtttcccactgttgctccagagaaaactagcagcaggagaacttctctgaaggtatcaagacatctttaaaaaacacttgttaagtgttggttcagctaaagcagggagttttcagttagtaatggcttttaaaaattaaaacaagtttagcatgtaggtcattaaccttgaatcactgtcatgattattattaaccatctgttctcaaatcgaaagatatttttcttttctagatcacatttattctcacattgctcaatttcactatatatcaagacatgaaaactgtaaaaatcacaccttctacattattatttttattgaaaaattcctaatgaaacagtgcgctctgggatagagaaaggaactaactgacattttgcttcttaacttgtttttatgcaagttctaagtggtttctggccatgtacataaaagacaaatatctggaaaaaaaactagcagaagtcagttatttggctctatctactttgagaattatgttatataaatgttaggaaattttttgtaatattcttatttagaaatgaaatataaaaagttttaaaaatatctaaggacagtatacagtcctaaagtaaagctgttaggtaaatgctacacaatcctcttattacagagtcacttacctgagaatataagaagagggcctcttgtttaagagtaaatgtgagctgcaatcaggattctgcactcatttggacacttagttttgtttttccatgactggtgttgcctgttactgagacacctacctgtcatgtgaccacagcttatgttacaatgtgtctagtcagacttagagatgtgtgaaagagcagtacctagacgggaaactatgggtctataaaggttttgccttcttgggcggagttcaaactaggaagccacaaaacttccagttgcattttcacagattaatgaaatatattttacacttttcctgaaagatattttatttgtgcaaaccttgttacaaagtacagccagttgattaatcgatgaagtgatttgtagtggattcttatattttgtgtaagggtatatgtgaggccctatatatgaggctttctatataatgaagtataattcagttcagcatttcaattcagcaatcacttattgggcctctactcagttgccttcagggctttataatttaattgataaagggaggttaattaattaattataacaacagatcgcttaatagtgtaactactaatttaattaatgacaaataacaatacattaaaagaaatgcattaataaaaataatatattggtgttatagacaataattttctgattaactttattattattatttcaatagcttttggggagcaggtggtttttggttatatggagaagttgtttaggtatgatttctgagattttggtacactcataacctgagcagcatacactgcacccaatgtgtagtctttcattcctcaccttcctcccacccttcccctcaagtctccagagtccattatatcattcttatgcctttgcatcctttagtttaggtggcagttataaatgagaacatgtaatgtttggttttccactcctgagttacttcacttagaataatggtctccaactctatctacgtagctacaaatgccattattttgttcctttttatggctgagtagtattccatagcatccacacacacccccctatgctttatatatatatgtaaatatatcacattttctttatccactcattggttgatgggtatttaggctggttccatatttttgcaattgtgaattgtgcagctataaacatgcatgtgcaagtgtctttttcatataatgacttcttttcctctgggtagatacctaggagtgggatcgctggaacaaatgattgttctacttttagttctttaaggaatctccataacttttccatggtggttgtactagtttacattcctaccagcagtgtaaaaaaatgttccctttttaccacttccatgccaacgtttatttttttattttttaattatggcaattcttgcaggagtaaggtggtatcacattgtggttttgatttgcatttccctggtcattaaagatgttgagcattttttcatatgtttgttggctgtttgtctatcttcttttgagaattgtctattcatgtccttagcccactttttgataggattatttgttttttcttactgatttgtttgagttccttgtagattctggatattagtcctttgtcagatggatagtttgcagatatttctcccattctgtgggttgtctgtttactctgatgattatttcttttgctgtgcagaagctttatagttttaggtcccatctatttatcttttttgttgttgttgcatttgcttttggtttcttggtcatgaactctttgcttaagccagtgtctagaagagttttaccaatgttatcttctataatttttaaggttttgggtcttagatttaagtctttgatccatcttgagtggatttttgtataagttgagagatgaggatccagcttcattcttctacatgtggcttgccaattatcccaacaccatttgttgaataggatgtcctttccccaccttatgtttttgtttgctttgttgaagatcagttggctgtaagtatttagctttatttctggattttctattctgctccattgatctacatgtctatttttatagtagtaccatgctgttttcctaactatagtcttgtagtatagtttgaagttgggtaatctagtgcctccagatttgttattttttgcttagtcttgctttggctgtatgggctgttgttttgttccatgtgaattttaagattttttttcttgttctttgaagaatgatggtggcattttgatgggagtcgcattgaatttatagattgtttttggcagtgtgctcattttcacaatattgattctgccaatccatgaataagggatgtgttttcattagtttctgttgtctgtgatttctttcagcaatattttgtagttttcctgtagagatcttccacctctttggttaggtatattcctaagcattttttttttttgcagctgttgtaaaaaggctcaagttcttaatttgattctcagttttgttgctgttggtgtatagcactggtactgatttgtgtacattgattttgtatctggaaactttactgaattaacttatcagatctaggagctttttggatgagtctttaggttttctaggtatacaaacatatcatcggcaaagagcaacagtttgacttcctctttagcagtttggatgctctttatttctttctcttgtctgattgctctggctaggatttccagtactatgttgaatagaagtggtgaaagcaggcattcttgtcttattccagttctcgggggaaatgctttcaaattttcccccgttcaatataatgttggctgtgggtttgtcataagtggcttttattaccttaaggtgtgtatcttatatgccagttttgctgagggttttaatcataaagcaatactgaattttgtcaaatgctttttctgcatctattgagtttatcatatgatttttgtttttactcctgcttatatggtgtatcacatttattgacttgcatatgttaaagcaaccctgcatccccggtatgaaacccacctgatcatggtggattatctttttgatatgctgctggattcatttagctagtattttattgaggatttttacatctctgttcatcagggatattggtctgtagttttctttttttgttatgtccttttctggttttgatattagggtaatactggcttcatagaatgatttagggaggattccctctgtctctatcttttggaacagtttcaatagaatttgtaccaatttttctttgaatttctgatagcattcacctgtgaatccatctggtcctagactttttttgtttcctgacattttttctattattgtttcactctcactatgcattattggtctgttaataatttctatttcttcctgttttaatctaggaggtttgtatatatgcaggaatttgtccatctcttcttggttttctagtttgtgtacgtaaatgtgttcacagtagtcttgaataatcttttttatttctgtggtatcagttgtagtatctcccatttcatttctaattgagcttgtttagatcttttttcttgttttcttggttaatcttgccaatggtctattgattttgtttatcttttcaaagaagcaggtttttgtttcatttatcttttgtattgtattttgtgtttcaattttatttatttatttatttatttttatttttattttttgagatggagtctcactcttgttacccaggctggaatgcaacagtatgatcttggctcactgcaacatctgccttccaggttcaagtgattctcttgcctcagctgcccgagtagctgggactacaggtgcctgccaccacacctggctaatttttgtatttttagtagagacggggtttcaccatgttggccaggcaggtctcaaactcctgacttatggtgatccgcctgccttggcctcccaaagtgctgcgattacaggtgtgagccaccacactaagactcaattttatttatttctattctgatctttgttatttcttttcttctgctgggtttgggtttgctttgtcttgtttttccagttcctagaggtgtaagctcagattgtctatttgtgctctttcagactttttgatgtagatatttaatgctatgaactttgctcttaacatggcttttgctgtatcccagaggttgtgataggttttgtcattattattgttgaattcaaatatttttaaaattttcatctttcttgatttcattgttgacccaaagatcattcaggagcagattattcgatttccatgtatttgtatagttttgagggtttcttttggagttaatttttaattttattccactgtggtctgagagaatacttgatataattttgattttcttaaatttattgagacttgttcatatggtctgtcttggagaatattccatgtgttgatgaaaaggatgtagttgttgggtaggattttttgtaaatatctgttaagtccatttgttctagggtatagtttaagtccatgtttctttgttgactttctgtcttgatgacctgtctagtgctgtcagtggagtactgaagtcccccactattattgtgttgctgtctatctcatgtcttaggtctagtagtgattgctttataaatttgggagcccaagtgttagatgcatatacacttaagattgtaaatttttcctgttgaactaattattttatcattatataatgtctctctttgtcttttttaattgttgttgctttaaaatcttttttgtctgatataagaattgctattctttctcactttgagtttccatttgcatggaatatctttttccacccctttaccttaagtttatgtgagtccttacgtgttaggtgagtctcttgaagacagcagatacttggttgatggatttttatccattctgccattctgtatcttttaagtggagcatttaggccatttacattcaacattagtattgaggtatgaggtactgttctattcatcatgatagttgttgcctcaataccttcttgttgttgctgttgttaattgtgttattattttatgggtcctgttaaatttatgctttaaggaggttctattttgatgtattcaagttactgtttcaagatttagagctccttttagcatttctcagtgctggcttggtagtggcaaattcagcatttgtttgtctgaaaaagactttatctctctttcatttatgaagcttagtttcactggatacaaaattcttggctgataattattttgtttaagaggctaaatatagggcccaatctcttctggctagcagggtttatgctgagaaatctgctattaatctgctatgttttcttttataggatacctgatgcttttgcctcacagctcttaagattctttccttcatcttgactttagacaacctgatggctgtgtgcccaggtggtaatctttttgcattgaatttcccaggtgttctttgtgcttcttatatttggatatctagatctctagcaagactaggaagtttttcttgattattccctcaaataagtccttaatgaccccactatataacatgaaatatctgttattggtactgaggtgctggccacaaacaattctgtgtgtcctgaaaactcttcagaatattcgtcatctttagcacttgttatcttagtgtttgggcttggcttagagtgatacatctcataacagggcaacagaaagaaccaggaaccaagatttatataacataagtcagtaaaactagaggcaccagaggtttacatttacattaggttacattttctaacaggtagcaaagcacatgaatgaagttcagtggaaggccttcctcaggaatccagtaaaaaccaaacatacacacacacacacggacatccgtgaggcaggaagggatgtccactatagtacagacaagcatcctggaaggccatcaaggagtaggtgggtttcagttgcctcaggaatgtggcatggacccaaactaagtgagtacagatacttgtcattgaggagaagattcaaaatagcatcctaggtgtaaaaactgaggcacctggggcaggggaactaggtctctggaatgttggcttaaaagcacccctctcaggaaaggcctcatatgccatgcagggggttatatatgtgttgtgggacacagatggcaaggagataattctatgcaccaggctccactactaacaggtaaacagaccaacattaacagagacttaggtaaaaaggtaggtgcccagtggtcagttctcaggcacttccaagatgcacctaacagaaatgtaacttggtgtctattgtgtcctaggtctaacaactgaagagaagtgaattagtacctcttgtggacagagaaacaggggcagagacccattacaaagctgtctcagataggcatttgaagctgtttaagtatgtagaggcttaagtcaggctggttctgaaatgtgagagagggttaagcttcatgggaaatcagcagggtagtttgctattttttattataaccaatctcacaatagtttgggacatcaaatatcaaattgttgggaatatttatccatattagtctttttgccactaatatttaaaaatagtttacaatatacaacaaaaagttgtaaaatttccatctccacttaatcgatcttatgtaacccatacaatacatcaaatgtcctttccccactttatgtttttatttgctttgtcaaagatcacttggctgttagcatttgggtttatttctaggttctctattctgttttattggtctgtgtgcctatttttataccagtgccatgctgttttggtgactatggccttatagtatagtttgaaagcaggtaatgtgatgcctccagatttttctttttgcttaatcttgctttggctatgtgggctcttttttggttccatatgaattttaggattgttttttctagttctgtgaagaatgatggtggtattttgatgggaattgcatttaattgtagatttctcttggcagtattacccaggcttttcttattttggcaccctgtgctgctgtctccttttccttctttctgcttctcttaaccaactgttacctacacttcaatactttctgagggcaattcatcctccagtaagtctccctgaatcttctcttccttccctggcttattatatatccttcctcttggttcccatagcacctatgcacacttctgtcattgcacttgccaatttgttttataatgatctgctcatctgtctcctcacttagactatgagctcactgagagcaatggctgttgcattcaccttatatcctcaacaccattctgaaggcaagagaaagaatacccagaggtggagctgggaagctggttgtccaagtagtgaatgactctagtttgaattgaactctatagccagtgggcaatgtggatgtgttgacagttttttaacaggggactagtgaaaacacattttgggtttagaaaaaattgcaagtctgatgacatacataggagaagagattagagataggaatttcacttcagaaatttaaccacaagagcaagtgacagatcacggaagtctgaaccagactataaatgtgagaatagagaaaaaagttaacaatttgggtgtgaaagggcgagggagagaggtgtgaagaatgactaagtgtggatctgtttttaaggattgaatggaaatttgagcattttagctaatcaggcctaatattgagcaaagcaaaactcttgcaaattgttatttcaagtgtgggctgagaaaatgaaaaaatataaattctcacgttataacctcttccgtgtgtctgatttgatagaatccagccccattgcctccaaattccattgcatcttagaccagcaaacacaagtgaattctacttaaccccagaattctgtatgaaaatcttactgcctttttttttctaatcatgtgtcaaagtgtgggaagaacttttatttatgttttaataaattgtcagtataaccatttttacttgaaaatattataatttttcaagtaaacaaattgtttctctaagttgaaaattttatgatggaataaaagtatttttcctcaaaacacatagaaattttacaacaatattttagagttaactaaatgtttctttagtagtttagtcacttaaaaagtgatatgattatgaaaatacttaaactttgtcttttaactatttctaataatgctattggtataatttcatatttttatactgatcttttctccaaactttagtaaaacatacttctgtaaacccctgcccacaaaactgaagtccacatttacttctgaatgactgataagtttgtaaaagtatgcatgaatttcgttattaaattaaagtttttattatattttatgcacaatggtataaattattaaattaattttcaagcttatagaacattgataaagattgtcattagaaaaccctgagttgattgttatacattacataacctttcattggtggattagtgaatatgttatagggtgaccatgaatccaaagaatcaaagctggctacagcaaacagagggtcaaaaggatatggaactatgcatgatccagcaaaacactcaatatctgttttcctggaatgttaaaagacaaagaagaaaacttggggaacactagatgcatatagttctggttctttaagaataaaaatatgggccgggcccggtggctcatgcctgtaatcccagcactttgtgggaggccaaggcgggtggatcacaaggttaggagttcaagaccagccaggccaacatagtgaaaccctgtctctactaaaaatacaaaaaaaaattacaaaaaaaatacaaaaaaaaaaatagccaggtgtggtgacaggcacctgtattcccagctacttgggaggctgaggcaggagaatcacttgaacccgggaggcagaggttgcagtgagccaagatagtgccactgtgctccagcctgggtgacatagtgagactctgtctcaaaaaaaaaaaaaagaataaaaacaagaatggtcagagtcctagtaccttgtccagtgtagtgctgccttgagattgcattgcaatctgtctgagagatagtaaaagaaagtgataccttccttagccctgtttctctttagactatgctttcccctctccaagttaatatctctcagtctaaagcctgggaaaaggtgccaattttgtttttctttcttcctcacacctcctagaagttacactgggacactattacttttttccaggctttggccatgtgtattgttttggagagtcaacttccttttttctttcattctgcaaatagttttgagctgtcactctgtactaggtgctataaaacttacaggtgcattttacatgcctatttcctataggccacgatttaacaaaatgttcataaatgagaattaggagtgcatgtattgaatcaccacacattaactgaacagctttcattggccagagactatattgacagtggagattcaaagataaactagagaaatctcatgcttaaataactttctataataaattatataagagaagtaggttcagggatcttgggagctcagaagcaggatgagttaaacaaaagttggattttgcctttagcttggtttcattatcctgaaggaagagcctgaaatatagtgtagggtgcaagtagtatatgtgggtggcaatctcgggaaacaggagcatgtgatgaataaggagaaaaagccaatataaaggtactgcattgagggcaatgagggctctaattctctgcaccttctcaagcattgtgcagattggttttctggattatcagcctgaaggacaaaacgaagaaacagccattagctcctgtctcccattgtctgagagctgccactaggatattaacttcctgaaattctgcagaaatctcctcttactttggcactggagatgcccatacgcagaaagcaaaaaggcacagcatatttaaggaagctcataagaaacagtgcatccagaagtggcgagaattggaggaatggacatgagactctaagaaccagcgcctttgatgttccttttgatctgttatgtagctcttcttgtacacaggtgagcaaaggcatgctggacaaatggattcacatgtgctaaagcatggggcaaaaaccacatattaattcaggaaaagacaagatgcgtggccctctctgtctctgtctaagggtgaattaaagaggggatatatgtacagagtggcagggcaggacttgagataagaaggctaggtgggtgctctcatgctagtagcattatagtacaggtgatgagaagctcctgaagaatcatcttaacatttgtattttagagcaacagtattgagttctgacttagagacagcaaaactaaagacagaaagactattttgattattaatgatgtagatataagaatatcgtcaatgtgaactaaagcatgaagctacttatgatatatcattaaaaggatttaactgattggagacaaacgagagggatggggaaaagaattcatttgtttttagttgctctttttttcctacttattcctttgttccgagtgtgaataaactttgtaaacttttatactaaaacattctgctcattcatacttatttctttgatgaaacaaggaaacccttgtatagttataaacgtgtgaatcaatttaaatattaggaaatttttttaaataaagctagttttctgaaggggaaaaacttggttcaattttttgctggcaatctgctttgtgatttttgaacatgatatctacatctagactcatgttttgctagctggaattttttttcaaattaacgctaccattattatatgctttactatttagcttttgcagccttggaaatctatgattaatacaaataattctctatggcaattttaaaaatacatgtaaaagccttcaatctacattgctactgtgtcgtagcacaaaaaaagaaaatgtgatcaaattttaataaaatctacaatttattcccttctaaatacagtcctagctcaggagaaaggaagctatttgtatttttcagaatcaaatttccctaaatgaatatagagaaagaattataactgaaatattgttgaaacagtggtcatctcaaatctgaaggtcattccaaaaaagtttctgagttttcattgcctcaatctaaaagttggcctttttggtaatagatgaaagtaaaataattgaaagggtctgttgcagttttggaatatcttgaaaatatagtagagtgaagccttcttcccttaaataaaagacaagttgctgattgttttctttctagccagataagaataatgccttctttctcttgttagtcttaacacctcacttgttactatgtgtcagaaaggcgagacaccataaatggagatactactgatggaggtcatctgacatggggctggtaggcagtgggaagactggtatggacacaggtggcttaggggttggggaatgatatggaactaaggaaatgataattagcagaacccagtgtgcatgtgtgtgcattcgtgtgtccgtgtatgtgtgtactgtagcacaatgcaagaaagaaaaaacaaggcagacttttcataatttcagggataaataaatcctttatcacttcatgtagaatattggctacttggaggtatatctaaacgtaaatatataactatataactacatgctaattaaaaacatacaaagaagaagtgcctaaagaattacaacagaaagtggcatagtgattattagagttaatataatataaataaggccaggcatggtggctcatgcctataatcccagcacttttggaggtcaagttgcagggatcacttgaggacaggggatagagacaagcctagccaacatggtgaaacccatctctactaaaaatacagaaattagctgggtgtggtgatgggcgctggtaatcccagctactcaagaaactgaagcaggagaattgcttgaacccggaagctggggctgcagtgagccaagatcgcgcactgcactccagactgggtgacagagaaagacccggtctcaaaaaattaaaaaatagtataaataatatttcaaaacacaagtctgttaagataaaaggtacagaggaatggtgagatgacttttttatttgtgtgataagggactgttttctgtgattgtgagaaagaccaggagttaagaaaaagtggccatcaataaatcagccacttatggggaagaaccataaaccactctcagatgaaatacaaatgcagtcattatttaatattattggaatatttgtattagtttttggtatgtgctgctagtgctggtacattttagtagtcaattaatattttgttaatcttaatttctaactaaattccagagtgaaatggaaataataatgaaaaaattttatttacaaaacagattttgtttttttctgttaagaatgatacacagttgtccttcagtagccataggggattggtttcaggacctcccttgggtactaaaatctgcagatgcctaagcccctgttataaaatggcttagtatttgtatataacctatgcacatcctctcatatactttcaatcaggggtccccaaccccagggccatgaccagtactggtccatagcctgttaggctgttcgataccaggctgcacagcaagagctgagctcctcctcctgtcagctcagtggtggcattagattgccataggagcacgaaccctattgtgaactgcacatgtgagggatctaggttgtgcgctccttatgagaatctaatgataaatgtaatgtgcttgaatcatcccaaaaccattccccttcccctcaccatccctgtccgtggaaacatttcttccagaaaaccagtccctggtgccagaaaggttggggactgctgctttaaataatctctagattactgataatgcccaatacaatgtaaattctatgtaaatagtttttatactatattgtttagagaataatgaaaagaaaaagtctacatgttcagtttaagtgttgataagtgtgtagagaaaagggaacccttgtacattgttggtggaaatatagattggtgcagtcattatggacaatagtacggaggttcctaaagaaattaaaattagaattacctaagacccagcaatccctcctctggatgtacccaaaggaaataaaatcatcacctcataaagatatctgcactgctatattcattgcagcattatttacagtagccaagatatggaaaccacctaggtatgtgttggtgcatgaatggataaaagaaactgtggtatatgtatatacaatggaatattattcagccttaaaaaaggagaagaccctgtcatttgccacaacatgcatggacctggaggatattaagctgtgggaaataagtccaacacacatccacacacaaaattgcataatctcacttatatgtggaatctaaaaagaaaaagttcaaatataaagttagaataaaacagtggttaccggccggatgtggtagctcacgcctgtaatcctagccctttgggaagccgaggtgggtgaatcacctgaggtcaggagttcaagaccagcctgaccaacatggtgaaatcctgtttctactaaaagtacaaaaattagccgggcatagtggcaggtgcctgtaatcccagctactcaggcagttgagaaaggagaatcacttgaactcaggaggcataggttgcagtgagccgagatggcgccacttcactccagcctgggcaaaagagcaaaactctgtctcaaaataaaaaaacaaaaaacacagtccacacactggttaccatgagtgaggtggcagggaggagattgggagatgtagatctaaggatacaaagtagcagatatgtaggaggaactaaaaagctgacatgcaggatgacaactatagttagtaatagtgtattgtattcaggatttttgctaattgagtagattatagctgctcttgccacaggggaaaaagtgggtaactacgtgagatagacaatggatgtgttaatttttgtcactataataaccttttcaccatatacattcatcttataacagcatgttgtttactgtaaatatatacaataaaatttatttttaaatatctgagtatgatttgatgatttgtgaaaatagagtgaattataataattttaaatgtaagttaatgttattagaaaagaaacagaaagaacataccacacagaaagtctgtctgaaggatctttgttttctccaccaatacaagtgttcattgattcagaggtggattatgagatatgaccataaaacaaaaatttcaagggaaatatattttattcaatgaaaaattctcaacacaactgttatatgccagtaaacactatatcttttaaataacaggtcatatctattatatttaaaattcaaggagagactacattagagatgctattagatcaacttctaatttcaaagatttctaagatatggaacagttactccttatacaaattaaaaaagcaaatgctgaagaaattcagctacatggatacaccatgaggtggaaagatgctccataactcttagttaaactgcactaattacacataaaaggaaaatgtttcatttcactgtaatttggaaaccaaagaaagaaaagactgaatttttacatactgttaaagagattgcgtatctgttctaagtttaagacagaggcaaaatgtattttattcatttgtcctgcaccgtttagaaataaaattcaacttccttttaattttttttaagaataaaaaactcagtctaaggaaagtcttaaagttttcattttaagtgatccactgttctagaagtttaatattttgtttaaaatgtttatgttctgtattccaccaagtctagttttaaaacaaaacaaacaacaacaaaatacttctctaacttggagtttaaggtgaaagaaaccaattacgtggtttggaaatgtcacacttttcatctcttttttaaaaaaatttttaattcaggacagaaattgtatggatttagtgtaagtcttgggatctcacaagtgtcagtatttcactctcctccatatcttgatagcaataacttgaaataggatctcagtagctcaagcaatactgggctctgagagttggttaaaaattatttggctgagcgcctgttgctgagggaagaactaatctcgagcatatttttggagccaaataccaaattgtttgtgcttagcaacacagcaccaggcttgcccttcagaatgattctagaccaaatgccagaaatgctctggttctgactacagagttctattcacaaatgacaggaggcaagaggtcctcctcactttcagaagaaaggtcctttgctttcttagtcaatggtaggaaaaccattgtggttttcattgcattacataatttttaaggtgattacttcaataagaagtgctctgtgtatatgtgtgtttatagacgcattttttaaacactggagaatttctgaaagtagtacaaaccttgtaatgtcaagtagatgtgggaaaaagggagtttacaacattctctcctgacattgctctcctttggcatctgcatttttaaaatgttaaaaatgtttaaaaacgtgtgcttaacacttaatttggtgatagttgctgttaccaaggcaactctgtaactccacccagataaaaataaatcttgaagatgagtttctgtgtctctgagcaaatatttttgtgaatagtagaagcagagaaagttaaagatacctgagcttttgatctttactagttttatagatatgtttatagttatacatttttattcatacattttagataaataactttgtaaagcaattgattcttcttgtaaaaatcaagtatattcttaatagactgataaactttctttttttgagacagagtcttgctctattgcccaggctggaatacagtgccatgatcttggctcactgcaacctacctctgcctcctgggttcaagcaattctcctgcctcagcctcttgagtagctgagattacaggtgcatggtaccacaccccactaatttttgtattcttagtagagatggggttttgccattttggccaggctctgagaaactttttaaggtctcttttgcagccagctatttgtctaccttatttcattcttaatctcactagccaatattttttctgtttaagtgctttcagcaaatattaaatgcttgtgccttcagtcttatcctgtggaaacactggtaatgacaaaaacacatatttcaacctaatatacaatagaaacagaatgccagttattcatggaggagaagaatagacttctgtatttaaaataacattttgctctgtgttttaaaatcattcttccttcatcaattgtaagcatcttgactataatttatacacctaaagataaataattcagtagcaatgataactgaaaacaggacacatacaatgaactagctaaattaccatacattctcatccatttcaaaaatagctctgtacttttttcagattttgttagaagaatattcaatacaaatttttattcaatgaacacttcagatgtcaagattgttacccacatggacaacagtaacctaggtaaagattctgcagccaggcgtggtggctcacacctgtaatcccagcactttgggaggctgaggcgggcagatcatgaggtcaggagatcgagactatcctggctaacatggtgaaaccccatctctactaaaaatacaaaaaattagccaggtgtggtgtcatgtgcttgtagtcccagctgctcgggaggctaaggcaggagaatcgcttgaacccgggaggtggaggttgcggtgagccgagattgcaccactgcactccagcctgggtgacagagcgagactctgtctcaaaaaaaaaaaaaaaaaattttatacctgggctctgtgctcaccagcagaaggggtaacatggcttcttaggacaaccttacttgaccatttacttctttgacactaggggtattcttagatcagcaggtccttccctccacttatgcacatgaggctcacagagagtctgggaggcagggaatttatgattggaaacagtatactttttatctaagaaattattaatgtcactgcattcaagtgattaacaccatcaatatcttcaagactaaggggattacatgatgtgtaaaattagaaaactgtcatctactagtggctaggcactttaattatattaagcatgcaacaagagaactcttcaaatgaatccatctctcctctgtattatttccaacccttggatccccatctgtttctgcagacaacagctatgctgctgaatgtcttaatggtttgctgccccaactagcttcaagatactgcaggtcaagcatagcatcttactcttccctgcatctccagcacctctcagaatgttggtcacatagaagatgtttgctgaggagttgaataagaatatgtacaagggacacaattagcattgtttaaaaaagatgtaacaagatagggtaaaggaaagctttggaggataaatctttagaacaatcaataatatcttctcctctgttggttagttgcccttcaatctcagccactgaatcaaatacaacataattactattctgatatgttcttgaatcgaatatccaataataagatattcggatgcatagccatgtctaatatcaaagcccatgcttttcgctattattgtactccatacattagcttccaaatttatttgcaatccaaatattaaaagcaagtcataagcttagtatcgccaatgtgatactaagtatccacttactaaactttattttcaaaatgtggttttatctcagtttaatgaacacggcatgttttaatttacactttcatattatatagtaagggcgtggttacagatatgttaatttcctgtgctgcttcacaatgatggaacataatagcaaatgaaactgttaatttgcagatacccataggcctttggtgtctgaatagaaataaacacacctacaactgagagaggaagcatgtgaagcattccagtgaacagaggccatttattcagtcacagacacaggagaaaaacaacaattaaaaaaaaatctctgatgaaaagttcataaaaagttcactcagtttaagcatatgtcctataactacttaaaatagagttcttcttaaatatcattctttgctgtttttagatttcttctgcctgtatcaaattaatagaacacagcatacttttaatttgctctggtttcttagtggggcatttattaaacacattaaaacaatagtctcagggttttactgctgatgttaaagttctgctttcctacttaccaactgtgtcatcttaaggcacatactttgcctctctctcaaatctcccaaatggagaatgataagaatacgtacctcaattaaagaagctataacaagtagaatgtttggaaaagtgccgggtacaccataagcccactatgagtattggattgtattacctctgaaagctgcagaatggaattctcaaagttatatgtccctaaaatcctcttaagtgacagaaatggagaaattagcagtctgtctaagagagcttttctagagtctgggcatatgtttttaggacaagacagttcagcttcagcttaaaatgagagagcacgtctgtgtccttactcctgggtgccaggtttcttgtccccatcttaagacaaataattttggtggagaagaggcagtctctttgatttcgctctaaaaaccttttctggaggaggtagacactctccacccccgttttgagactcatgcagctgaggatgactggctgagtacaagcaattgttccttctaagcagtttcaattcttataacttgtggagatattcttaagtccaggggattttgtgtatggtggatttttattacaaagtcctgtacttcataggaacaaaataattcaaagtcaggaaccagatcaaagccacaactcagatatggcaccttgagaagttcatttgtatttcacttgcataaaaaccctcaccactgctatctgattttcacaaatcattcaacagctatccatgaagcacccactgtgtgtctggtctctgtgtcagtccctggcttcatgtgtctttccttctgtaccctgactccccaactcatgaacacatgaagtaaaaaaatgaaaatctttttctgacctctcttcaaaatcacttttttcaaaacaaacacctctcacctgctcatcctccagccagtaaatcacaggggcctagaaatgtcacttacaaatattttctgattctgtccctcccttcaagcttgccaacattatcacagtttagggcctgctcatctttcccccaatctccaattagatctctccacaatgcaattctgcacattccctgttacaacccttcaattatttcccagcccatccaaaataaaatctaagcctcttactaacacattcaggaactctgtggcctacggttttctacagactaattttccagcagttgacttccagtgcaagtgaaaacctagtgtcatgcctgcatgatagataaatttgaagctgaagagcccaaatgtatagaccatgccatgaaaggtttatagtcatgacacagtggccctatagtacagtgcttgaagctggctctctactgtcagacagaccacttgccagccatgagacctggggcaaaatgccttaatttttatgtgcctcaagttctcatgtgagatgagaataaaaattacccctatttcataagatttgataaagtgtttagcataatacctcataacaattgcaattcagtggtggttattattataaagaaaagatgattaactttatcttaatgtttaacttgttctgatagttattgatctatagctttgatatggaggtttgagaatgacctggaaagaattggccacaatgattgaagatagtgatacaagaataaaagatgactgcaaaatgtaaacctgcaataacagaaagaatgaagtcactggtctcatgggaactgatatgggagaaaaaaacagatcaaaaggctattcatgttttgggcctctttgtcaaaatggaaatgagaaactggggaataaaaattaaagcaattctagcatctggttttaacataattcttatccctaaaaagaatctataagaaactcccaaaatgacaggcagccgtgggtagcattgcatttcaagtaatcttttaattgttaaaatttaagtttccaacatgaacataaaattttcaacctaaaagaaatgagttccaaatctgagacaagtgaaaaaggataaagcctactagggggtaaattccatctctttagagatctagtacccaatttagcaatgtccaatcaagcctttaactactacatttgaacacctcatcatttcaaaatgttacttaatgatgccaattaactgtacaatgtctctgcatagcacatagccctaaaatgatttgtgcaatgttactgtcagtaaaactgaactacagggaatgctcatattctatgtcattatatacagaaatgcaatatcaataaagtgatatctgttggtattagaaaaaagtgaaaattttcatatctttctattttcttttttcctcaatgggatgctcttgttaaagatagctctgcatagtaaggtttgtataaacattatttagctaaagttaaaaggggtaacatactggttctagcacagatattaaaacaaattagtttgtaggtagggcagcaatcaattatattactaaccatagctttggtccttttatcctttcccatttgattttacacagtgggatgttaaaggttgaatgtctttggtatctataaacttaattgaaagctgttatttgtttgtttaagtctgttgatttttataatcataattttactcctatagatttcttgtaggagtactatatgaatttatgttgcactgaattttgttatgttatacaaattaataggcttttatttatggaaagctactattgatctgtcatttcttaaaaaattactaaaaagtgttaaaactttaaatgttggagagtttatattttaaaagttacatgctagaaaaacatgatgtctgagtatattagaagttatagataattcatctgtcaactataaaactctccaacactgcctttctttaatgaataatatgaaatttagcagtgaaaatgtgacaatgtacaatcctaaataaatcaacaaatttagagatgtacctctaaaaccattgtaaattcaacagtgtaattttccattggactttcacttattcattcattaaacaaatgtttgtgagtgcctgcaatgtatgagacattgtactgaagctaggcagtgtgagttatcatatgggattatcctttaaatacttctgagggcaaaaaaaaaaaaaaaaagaagagaaaaggtgtgaggaaagataaagggttaattcattaaaaaataacacttgaggactgttttctttgcaaggcataaagttatcaccctttcaaacagtagatatttcacatttaggatgcgagactccagttccaacaaagctcattgcacagctgctaccctgattaaactgctacatgaactctgagcaatgtagcatggtagccgcatgcttctgcttgcatgatggttaattccttccattctcattagtgattttctgagctttgaaattctgatggtacctaggatataaagcatatttatctaactgaaaaacagataattagatgtaacataaaatatgaatggctttgtcactttattgtagcagagaatgaatgtgggataaattaaagctgatgctagaacatatgcctattttttagctggaaaatttcaagatttatgtactttgggcttgagaaagaaatggagtttattttttatgcactgacatctctttttttttttttttggaagagctctcttaggaatgaatggtatgtaaatacagtaggaatgtaattatagattttcctgacccagttcctaaataatagatatcatttcagaagtgccccaatacctgaccttttgctccaagccatatcaaagcacacatctagtctacttttcactctcattcctagccactatgacaatactattcagataaaacttctagtcctctacttatgtgactcataccaacttgaccttacgatagtgactgggggtgcatatctaggttcatgctgtttgtccattattatggttttgtgagaaaaggcaaaatttctaggtaaagtgttatgaggacgaataatccaccaggcaaccaactgaccctttcatttgccatcttgtcacttcaaacagctctccagaacctgcagccagcacagaccaaagtcaggtttgtctcctcttctgttgatgaacaaaggttgattccatatcgtggctattgtgaatagtggcagtaaacatggcagtattgtatgaaaatatcacagatagcccttaaatatgtgcaactatgatgatctatcaaaattaaaaattaaaatttatttttaaaagttcagttagaaagcttgtagttcctggcaaactactacctttctcggcaaaagaatttgatatctcttaaatattttctgcctaatgctgatagattgtatttacatattccattaatgcaataaataaaattacaccaaaacatcagcattatttatttccaggggcatctctcaaaataaattcctccaaaattcacaaaaccaaaaccaatgtgaaattgtactcagggatgcaaatgtagcccagtgaagcatttgcccacttgtttggtattattgaagcacaattagaaaaatgtgcaatgtatgcccaaaaattctataataagggccaggcgcggtggctcacacctgtaatctcagcattttgggaggccaaggtgggcaaatcatgaggtcaggagatcgagaccatcctagctaacaccatgaaacccagtctttactaaaaatacaaaaaattggcccagacgtggtggcgggatcctgtagtcccagctactcgggaggctgaggcaggagaatggcatgaacccaggaggcagagtttgcactgagcctactctccagcctgaacgacagagcgagaccccatctcaaaaaaaaaaaccataataagaactttttaatatactatattataatgtaaaaagactagatgtcaaacaaattaggtgatgggaaggaattgagggagaattttagactaagcaattgagcagcacctgtttttcaccacaaatctgttacatgtattgctcaattgtgctgaatccatattgggtcctggtggctatgtaatagtctctttcttggataaatgtttgtcctctcttatggtttactaatggtgtacagaacagcattgaatagtggttatttcctatgacttcctagatatctctctcataatcctgaatgttttaaagatcattcttagatagagtacagctagacacgaaccatagtggaaatcaggtagacaaaatttaaaaggagtcttaattgaaggtcattttattgtcctcagtattaatcttacttaaaacaaacctgtcactgagcagaactcaaaacaccagagccctttgccaaatgtgattttttacaacaggagcgctggcagttgagaggagtattctgtcacacttgagagaattcgagtccctgaagatttatatgaatgcttagctattatcgaaccatctcttcacagatgacttagtaaatgtctgcctttgcatcagataatggcttacaagttaatctcctcttgctccctgttacacacatatacaccttcttcctaaacagctcataaggtgaaagaaagactcagatttctgactatgtaattgataatatcacacggactgcctgctcatcatctgctagtcacattggcagagttgacagttttggagacactgaagacagtgcatatattaggaaataagcagtttcctgatataaattttcttgtagtttataaattacatagcatttattattccctcatattttataacatttaataatagaactgacacatatattcattttaaactcaattgtgtataataactatcatagcaacccttcagtgcctaaatatcaaatcttccattcctcccatgaacatcttgaatatataggtactgtggttagctccaacaagcttttggttagaattcattgcactgatacatagacattgttttaaaggcaatttcaaatcaaagctgtcagctgtgaatcaagcacaccttaaaaagtgacacatttgtcactagattccagcctctcaaattactgacacgcatcctttttatgtaaagatgacattgttctttcctgatatattgcattcctcatgaatttcttatagtcatagaatttttataaaccatttcagaatcgctgaaataaacatcaatatttttaactttttcattctgtcaaaaatattgtatgcagagatattgctgtaagtgtgtatacctgtgcttaagagactagggctgaagagaagtaatcaaccgaaccactggtgtaaatgtgcgtcacatttttagtgactagaaattgaaataattccaacaaatttatgtgctttgggcttgagaattcagactgccttaggctaagataaaaatcttttcctggtactatataccttcttttattgaatgactacctggctctttctattatatatgcagattttgtacctctggtcatctttgtaaatggtgcctaaaagatatttgaagaataagtgaccagcaataagaacaaatgtctatacaaaagcaccctttagttggatgtaattcactactttgagttgttaataacctctaaggatgacagtagctattagttgaataaaccattatgtctattattagaacactagatagtttataagtccaaacaatgcataaaatacctatctcatgttaccattgtttaggttaccagataattgttctgtccaattattccacttaattttttgcttgcccattagctaaatggcaagataaaatttgtcaaacgggggggaatgtattgaaaatgctagacaactacacttaaaatgaaaacaggccaggcgcggtggctcaggcctgtaatcccagcactttgggaggccaaggcgggtggatcacctgaggtcgggagttcaagaccagcttgaccaacatggagaaactccatctctactaaaaatacaaaattagccgggcatggtggcacatacctgtaatcccaactactggggaggctgaggcagaagaatcgtttgaacccaggaggcggtggttgcagtgagccgagattgtgccactgtattctagcctaggcaacatgagcgaaactccatctcaaaaaaaaaaaaaaaaagaaagaaaagaaaacaaatgcataatttgcaaatattatttttatattgtatgttatctagggcttctaaatgcattcttcttataagcctaggtttgcaataacattcatttagaattgagtaattttaaatataatattttataaaataaaatataataatttctcttaattctttgaaaatattaaattaaaagggggttgcaaactctgcattccacatttccatcccaacatttaattttagcaattttgtagtctgcctaaaatgcaatccatcatttactgtttagaaaatagggaatgtacacaaaggcctttcagctttccctgaactccataaaaatctttttgcttctttactgcccccctttgtcaggagttctgaggaactgttttttatcttaagtctcacaaagcatttaggagaatatttaaacttaaattcttttaaaacttatgttcaggacaaagtaacattgtatgcattggtgtcatatgtatttaaattttgaaatttttaatactggcaaaatgaggtttcaattttaatataaattatttaacaatcttaaatcattaaatatattacttaatatatttaatatatctaaacagtcacaattttcccatactaataatcataaaaaatcttacccaatggtcatatagatatacttaatggagttttgggggggtatttttgtatattaaaaaattcatatatttgccttacttagaagaactgattaaatgaaagtataatattaacaaacatattgttattttatatttgcatttgtgataattatatttgaaacgttcaagattttccaatgaatttcttttgcatttgcgtatttgtgcctttttattataaaaataggtggctttttagttccactgcataagtttcaacataggtctacaaatagtgcatctttttgaagttaatcattataatcacaaattgaagttgcctgagctccaattggagtctaaatggatgactgaatcttattattcgaaacccactgttgctacacaatatggccacacaagagagtacacaagacccgtctgattcagcctcagtgccataaatattttaatggtttcgttggaatctggaaatggagctcaccacaggagatgcttcttcctttgactctcattattatttcctttacaaattaattaataaaaacttagatgctaaattagcacttgatgaaaacttatatagccttgacattttgattctgtgagtgaataaaaatacttggagaaataaaaatcctaatcatgttcaggaatacccacaaggtaacaagtacatttttaaactttaaaaacatttattattcatgataaaacatgttgtgtgatttaaatataaatttttattatttgctttaacttatttccggattaaaaagtaaatgtttacctagctgttctaaatggtaatcctcatgattaaaacagcaatttgtcatatttcagttacaaatgatcttttattattagttatagaacataagtttcttcattgactgaggcgatgtttcaagtagataaatctgttaaaaaaattgtggtcatattctgttaaattctcataccaggcaatttgtttgatattcaggaaaaacctagccactgaccaaaaactctacctgccttctcagttgtatcctcttggacttaaaggggactgggaaagttataagatggttcatgatagtccatcaacatcccaagaacaaaaacagatgttgtactgacagcatcatatgatcatatgcatgtaagagcacattcatattgccaaatcagttggaatttttcacggttgaaagttaaatgaaatgcttagatgtatgagtcatcggagttaaagacaattacagccagatttatggctgtgctaaaataaagctagttagaaaacagaccaaattccatgacgataccaagtctgactaatgattcaccttaaatttcggagcaacatttatcctcacttgtttgtttatttgacaatgtgcccttatccattaagtaactaggaggaagggaaaagcactacgtgggtgagtgacaagacactgacactgatttgtgactttggataattcctggatgctgttatctgttttggcatagagatggatctgtaactgctaataattgccgactgtgaccatcccagaggccatttacttaacccaggtatttcagacctgacagcccgaggataaacacgatttccctccatcactaacttcatctgcagggcctaagcctccttcacagtctctccagtgatttattggcatctccaagggtatctcacatgtgctgaagaacaaatctgctcactttcatctgcttggttttcccttttgaaatctgctgctttaaaattactaagggaggaatcatgcctgctgctacccttgccagtgaccttgcagtttgtgccctgattgttccaattaccacaatcaaaacagaagcgtttgcagttactgcagtgctctctctgtggatgtcaggtctgactcagagagccaggctggggaacagccatttccactcttgtacctctgcaaaaggacttccatgttccgtaaacagactcccacctctcattttccccccaagcaaagcatcataaattagagagcatgtaacgggaaagaaaatccattagccatttgggttcagtcagacaagccagctcatggaaagtttatacaggaaggtcacatttcaattgagatcaggagggtgaaagggtccagctgtgtgatgagagagagaatgttcgggaatgtggaacagaggtatccaaggcagaacaaactcgtatatgaaggctttaagggtgtgcaaatctagcatattttatgacataaaagagtcctgattagctagaatatgatgaatgtgagaagaggtgaaggctggagataggaaaaattattccagatcttataagctatagtaagaaatttgcatattatatatagacttgtgggaagccattggattttgtaagaaggagattaacattatcttatttatgttatttgtgatttataaccccaaatgtgccagatacaaacaaaccaaaaataataataataataataagaagaagaacaacaacagcaatggaactgtggtgatggttttggtcacaaaatgcatatatatctatttttcacaatgcaaaaatatttcattatttcaaattttaacataaatgtgggtatgcatgagcttacaaatcttgaagtttattggggaatattggtgagcatggtttttattgcatggtcacaacttactaatgggaaacatctgaatacctattgagttaatgcatgcacatttttattttcctggaatactgagaaaaaggttgctacataatgtcttgatagcttctaagtcatggctcaaaagtgaatgtggaatctgctaatcggaatggactcagattcagccaagttctcaaaaacatttgctttcatagatgtcttcaagaaacaaggagtcttgaatttaaattgtgaagtgtctatcttagaatagagagatttaaaatctgactgtattttgtttaaaaaagcctatataactgtattatataaaattatttatactacagttaaaaaaagaatcccatcctatttgtgcctaaataagtgcctgcttgtagcatgaaaactatttgttgagggtccttagatcctcagagcatgctgtgaaagtaggtacaattgttctttctatataagcctcttaagataacagataattgccagaaatacagcacacagtacaaaattaccttgttttacttttgccacaaaaaacaatttcttttggctttgagcaataaagtccaatgatttttttcctttcaaaatatcttcctccctctccataagttttatatttattcacgaaggaatattccaatatcggatgtttttgtctgtgtctcttcctggaacaaatgttaattaatctctttgggtttgtatgtcaagtggaggggtggggattggggacaggtgatagttgtctagggagttaacttcatctctataggagagtggatagacgctgtatacgaaaagctcttgaaaagggaaatacagcagccacttcctcagggcttccatggtggtcagactccttgattgctttagattaactctggcttttgtccttcggaggccaccagattgggtggatagacattgtccttgctgttcttttgacctacctacttgtactttaggggaaaaaaatgcctgtaataggttaaatgctttctcaaagatcaccaaagtatataacacatggcaaatagacagagaaatgagacagtataatcagtataatttataaaagtaccttacagcaggatcccatgggatatgggttttttttaaaaaaaatctacctaatcttttcattgaactcctattcaggattcattatattgaatatggctcagagacctggaaaattgtttccacctttttaatttattcaccatcatttatggaagttttcaaggacgtttacttacctacctcagttaacagattgtactacttgggaagtctataaatatgagcttaaagcattttctgagttttaaaataatttagattgtgtagaatgttaaaactaaaagaggaaaaaattattcagttcctcagttgaacctagcaatttatcttttcacagtgtgctcaagtatagtttttgaaaagtaaagaagatggtttttatacaaacataaacacatttcaaagattttattcaactaattaattagtagtggagccaataagctggtaagactggtttaaaggaatatctgaggaataaagatttatagaaacagtcaaagaaattctaaagagaattgactaatagatataaatctagtaaatatttgattaataatagcagtaacctatggaattatgttttctactgagcataaatgagcatgaatctctttgggtttgtatgtcaagtggaagggtggggattggggacaagtgatagttgtcaagggagttaacttcatctctataggagagtggatagatgctgtataagaaaagctcttgaaaagggaaataaagcagccactgcacatctgcacatataacctgtagatctgggggctctaataaaaaagttaatggcaatgtcaaaatctggtgttttatcttagataacttcatagtcattgattgagccccttaaaaataacatttaaaggacatgtagtcattctgtttctttattgccaagttttcagcaatttttctcatgagaatgagtgctaagaaacttttggtggagcgtggtggctcaagcctgcagtcttgcactttgggacgccaaggctggccaattacttgagatcagtagtttgagaccaccctggccaacatggtgaaaccttgtctctactaaaaatacaaaaaaaaaaaaaagtgggatgtggtggcatgcgcctgtaatcctggctactctggaggctgaggcacgagagtcacttgaacccgggaggcagaggttgcagtgagccgagatcctgccactgcactccagcctgggctacagagggagactccatctcaaacaaacaaacaaacaaaaaagaaacttttaaaatataacaatagagacattacataggcccacaaaaccacctccaaaaaagcattctatcacctgcaagaaagcatatatatatatctgcttttgtgtatatatatatatatatatatatctgcttttgtgtatatatatatacacacacacacacacatatgtgtgatatcagcatgtgtatttacacatatattttgtgcatgtatatttttaactaaaaatgtgctaggagttagatatgaactgattttggaggaggtgatatgctgtagagagagagaatgggagaatagcagtattataatctctctccattgtattcagtttttttctttgtctgaatttttaatagaagtcagccagaagatgttagtttctgggaaatgtgttgagatttacagtcaaatccagagagaactagaggcttatgagtaaataagtaaaggttatgcagagaaagtattctttttcctgtgtaaacttgaatattggccaggcgcggtggacacctgtaatccagcactttgggaggccaaggcgggtggatcgactgaggtcaggagttcatgaccagcctgtccaacatggtgaaacccattctctaccaaaaatacaaaaattagtgggtgtggtggcaggatcctgtaatcccagctactacggaggctgaggcaggagaattgctttaacctaggaggcggaggttgcagtgagctgagacagcgccattgcactatagctacggcgataagagtgagacttcatctaaaaaaaaaaaagaaaagaaaaccttgaatatttcttgtacttgtgttcaaatcatacagttatgaaagtttacccctagctgttacacttaaaatgtacttctgaaatatacagagagatgatacagactattaatgagttccactaaacttttaatggtttagaaaatacaaatattttcttatttttctggaattccagccattaatgtaaaacattggtttcaacataaataacacactggcatgcacatatgcctaagcatgggcccccacacatacagacattctgaaagaccactttttaaaaatattcagtaccgtatattgtgcattccttctttatccacatacttaagctgctgcaagcatcccattgataacaccagtaataaaagatgggaccatcagtaatgagatttgaaagccccttttgcaagaaagtaaggactagaaggtggaaatcactctgtcttagagtcatatggattggggctttgctagaagtgtgtgctctcagggaaagctgcctttttattttctccagagaaaagcctttttgtcagtaaaagaagatgtatcatccaatgcatatgtaaaattctaaacagcagataaaacaacattcactattaatctctgcaaaagaagatatattgaaaaaatcctcaagtgtccctctttgggtttctttgttatatattaaagcagttatctttagatgcatgagaatcacctgaagaccttatttttaaaattcagattcctgtcagttcactcccaaagattccgattcagtagttaagagacaaagcctaggaatgtgaatttacaatcaacacctcaggtgatagccatgcatgttcttaatgctctactactatctatgcataaaaggaagataaagttttaaaaacttgaaatgtggtataacagtttagtattgaataatatacatttttacttattgtaacaaattatgatatctacttggggcaacagtatcttttattttggatctgaatcctaattttggctaggtatcactgagggattcttagtctaaaacaattaaatggagttagtggttttttttagtaactcttgattttctgtttttttccattggcatcttacaaaatttattcattcatttttccctttttcacttggcattatttgttagacagtggacaaaagaactatagaaagtagagaagcatgtgatgttgtcctgctcttagattctcgcaactcaggagaggacattcgcttacaccaatcatctcaaaacatggcagtttatgctgaactcagtccaatgggagagcatttgactgagcacatagggagagaagttagctctgttgaaggataatcaacgaagaattcttaggaaaggtacagtcattcattgaatatttgctcggcacttactaggtgcatatgtgcactaagatctaaggatgggctgatgaagaacccaggtcccttttcttctagtggacatgcagactggcctaaaaaaaaaaaggtaactggaaaatggataaggaaactgagtcactcggtttatttattatcactcggtttatttgcttttgtttgtattttcattttgacacagcacagtgtcatcttaacgcatcctccaaagtgaaggatggggtggataacactttagttggcatttctgtagccaggagccaggatctttctcccataattgcattaacctgggaaggcaccctctaggtagatttgtatagcaccctggttaatcaattatcagtttacttcttgtctcactaagctttaacaccttacatttatgaagcagtgtaaatataactttagcatcttgatcacagcaagcacctgatttgtatttttttattagctcaagtgaaatcagatcagagaagtacattacaggtcataaaatatgtgcaaatttcataatgacctccttttaaaatgtgcaaaaataagattgttaaggcacattccagagccttggggggtgtgtgtgtgtgtgtgtgtgtgtgtgtgtgcgtgtgtgtgtgtgcttgtcttttgagaatatctgtatatcagaaaatttggctgagaagcaatcttcttcttagtggttctttttctcttttgaaaataaagtactaaaaatacttaaagatgcagaacagcaacctgttcccagtgagactctcgtttaattaatgtggtgatctatatagagaaaagggacaattgcaaaagtccctcaataattatctaaccacagtctttaggtaattacagcagaaagattttcaagacacaaaacaccctggaaaatttgacctcttattttgattcaggcctttcatttcttaaatattttctttaatgttgatgtttatgcttgacaaggtcagcctaatgccagatgaatccctggaactcaaaacattgctgaattcacagttgaaggattttaatataatataccagcttttaaaaatcctacagtgagaataacaggactgaataaaaaaattaagaaatgctcaggtagaaataaatagagaaatttagaaaaaaaataaaacgtattcaaaataagtattaagcattggcaaagaaaaaatagtagcagacaattacatgttccatttgtaaagatgattattaattagtggtcttgcaaaacattggagaaaatttgctgaaccatcacattcataaatattaaaaccacccattagtgaaaatctttttactaaacttcacaactgatagtcaaataatgttcagtttttctccattgcaataaaaaataaaggcttttgccttcagatcagtctctgggccttattaattcagtcagccagaagccacatggaaatattttgttttgttaaaagccagcttgccctcatgatcttttaaaatcttttaaaaatcttccatcagccctctccctgacttgaattatggcagtgctttctaaactggtaaactcaatctccttggtgtgcctcaagatagagtacataaaccctccttagaaattgagctctcaattctaaattgcactctccatgagagcaagcaagaatgctttgctttgtattaagtggtcacaatattaaatataaccatagacagcactgtattttctaaacaccttattttcttttaatgactgacataaattagatcataagtatacaaatgcatatctgttgtatttttcagcaccatgtgtttttttttcttttttctgagttattttcctgctttcggcagccttttctctcaggtgccttgtgatccacagtggtgtgtgttcacactaaccaaagcaatagtcttacctgccagaaatagctgtgacatttaaagagaggtccaggggaaggcacagtgcttaacatccaagtctgaagagctaatagtgaaattggggcatcagctacagagagatttaggggaagtaacaggcaggttaaatattttatggaaatgatttctgttctgtatatgattgcaattaacacatgtcaatctgtttcattaatttgttaactcatctattatgctatgccatgaagaaaataaaattggagttctttatttttttgagatggagtctcactctcttgcccaggctggagtgcagtggcaggatctcagctcactgcaatctccaccacccaggttcaagcgattcttctgcctcagccacctgagtaactgggactacaggtgcgtgcaaccatgcctggctaatttttgtatttttagtagagatggggtttcaccatgtgggccaggctggtcccaaactcctgacctcaagtgatccgcctgtcttggcctcccaaggtgctgggattacaggcgtgagccaccgcgccccgccacaaaactgaagttctaagcttcagtttagatgctcactaaatgcttgttttgcaatacctgactgtaactggcaggaatatgttttgaaagtcctcattttccaggtatgcagatgaaatataggggcattatctactatgtcaaattataatgatttatcagtggcacatgaaagtcgcctcacatttcttaatcagtgatataccattatgtcatgccaccttttaatgtaatatgtttacatctttctttagatgtaagcattcatttagttcatcacggtggctttcacacttactccaagaacgctatgagttcctttgatgtgctcaagtctcctgccccagggagaaagggagtggtgagcaggaatcgctttaatctatttacacagatattttcttttccatttattttaaaggaattttttttaacttaatgagtatgcagtgacggtggtgatgatgatgatactaaggtttaaatgattagatagtcaaatctgggctggaattgtaatactgttttgacttttaatcttagagaagctccagtctgcttattttctgggcataaacacatgagaacaataacacagttctgttatctgaatgttgttatattttgtttgaaacattcagtgactttcaaatattgtatttgcctaagaaaattcaacagagtcagacattctcttccaggttaaatttggtgagtctgctaggaaaataaattttgtgcactggtcattctgatctagtggacgttctaataaaagcacctttgtgctgcctacgtcttcactttaaagataagatacctgggtactcgacaccaaattatagtttgagatctcaaaaatgggatagggaaaccacagctcaaaaacaaaaatactagcactggaaaagatagaactagtgaagatgaatcattctctagactttaaattcagagatatcaaaattaagaaaaagtaggaggaataaaaaaagagggtaagcaaaacaatataagtttgtatagcaagagggtataaagcaaatacaatatttttcagaaaaattaaataaaaatagatttacataacattgtttttaatctcaaagatcaaatttcaattttcatctcattttaaaacccatatgcacagtctcctttatatacatcagttgggtgtcaaagtgacttttttcttgtttccaaatacagttatttttaaaatttaattgtatgatttaggaatttgaaagcaagccagtttgcacacacatatgttattatatgtgtgctttagacttggtttttagttaatgtaacatgacagggccacctgagttatttgtttacaaactagctggaaagccaccctggaggagaaacctggcaacaaaatggtctgcagctttgttattgttatctataggattggatgccattattgctgtaaaatagttcacaagaactcagtctatgggaaagactcaaaaattctttgcctgttaaagaaaaatcaggatattggactggttagtttaactaaaaagtgatgatactcagattctgcttggattcactgcttctcagcagttgttttgtttctttctaattgatattttatttttcagagaacccattataaaactcttcttcttcccttaaaatcacaaccacacaacagcaattaaaacatgctttgacgtaagactgatatggttttaaacccagcttgactatcgaattttttactttaggcaaaacacctctgacatttatgtcttatcgtcagtaaaaaggggtgattaacagttttacaagattattcaataaataaatataaattcctccttttccttcctttcctttcttcatcttcagcatctgcatgccataagctcattttagttctctggactcatgttaacatgtcccacctttcccaaattaaacatcatctctgttattggctccattcttttcctctcatttgagacaattctttatcaaccaacaccctctctgctctgtattgtgaaactctgctcctactacattaacagtctcttggtttctttaaaaagaagacaaaacaattaaagaacagaagcaaaaaatctactcaaatccccaattgttaccctcaaaattaattgtcccacccctagctttctcattgcacaactctttgtcaaaatgttttctaccatcacagccttcaatgatctttctggttcctttatctcctgaagtctgacttctacctccatctttttctggactattcaacacactttgagaaaaaacatacttttgttaaacaggtatgcatccctgaagcataaaatacatagtactgaaagtgcacatgtgtggttcttcccattttttttacagcacttgaaactgacaagtagtagtaccaattacttagtaaaagacctttttcatttcatttctgaaatattgttattttcctttttcatcttccatctctgactacacctccaattttacctctttgctgccttccttcctaagaaagttcttcatgcaatgccatcttgtttttcttcacttgcctctttttctcactttaattttatgaactctgatgacttacctctgtagtgtaactactcaaaatatgtatttctgaagtctcaactccaatctcatattttcaacttatatttatggaggcatctcagactcaacctacctaaaaaatggcttatctgccctaaaatctactttgttctttttttctctactgctaataattatcttcctagttggtcaagctcaaaacctaatcatttttactccttgtccctgtgtcagctgtccacattcaagcagcgtatcatttctgcacatttttcaagcaagtcagtaactgccttttgtttgggactgtcttttcatatagtgaacagccttggaagatagaaatcatttctccttctaaaacaaaaggcaggtgtgcttgcagccttggatagaggtagtgcctctttctaaagcaaagggacatctttactggccattataaaatatccatgtttcctgagctctgcgttcctcttttctaatgcaacccactgagcatgtaggtgtcacctgagcttttctgtgggaattgcggcttgaggaatcagtgcaagaaaatcatgatactcttgctaatgctattaatgtgagtagtaaagttaattgtctctgacccagcactattgtgtctttgcccagcactcaaaagactggcaggcttgcaagtaggacaaaatgttagatttttcacagttcttctgcttataagtacttgttaaaaccaattaaaacacaacttgtagtttgcacctataattttgtagcatttgcttcttatctatgtcactaggatgtgcttagtgacagacccatctatcatctattactcaagtttttggctgtattcctaggcaacagagagaaggggaacaaacaagaggacctgtgcacagtttgagaaaggcaaaacaccgagcttaattgcagacttgaatgtagctagcaaacgaagtaaggcaaaaggttccttttttttttttttagatggagtctcactctgtcgccagtctggagtgcagtggtgctgtctcggctcactgcaacctccgcctcctgggttccagcgattcttctgcctcagcctcccgagtagctgggactacaggcatgtgccaccatgcccagctaacttttgtatttttagtagagacggagtttcaccacgttggccaggatggtctcaatctcttgaccttgtgatccgcccattcggcctcccaaagtgctgagattataggtgtgagcctccgttcccggccaaaagtttccattttttaaatagttgggtttttagtttcgattctttccaaaaaaaggttttcttaaaaaaataaaattagcaataagatgaaatataacaacaatataatcttattaagacaatatatgatatacatttatcaaaatacttatattttcaaaagtgcttaaaataatctagcacatagtagatgctcagtaaatatttgatattatgactgtgcatgggtcattataggctactttatgtatatcatttcatttagtacaacatcactctgaaaaatgttttattgttaccgtttttcagttgaaacatttacgttgctcaagatctcactggtaccatctactattaggtcagtctgccaccaaatctcatgctcttaaatgccctttttctcctgagcttccaacaaatagtgtactgtatataattgttgaagggaggggactgtgagacaaaatatttagagtgaatgtgtagccacaatttcagttcctcaacaaagtgataaaattaggaatcatcctcaatatatattcttccaacacacacacacacatacacacacacacacacacaaataccacaagcccacttgaatgcaccccacctacacattgcaaccatagagacaattgcagcattaaatacagaatattctgtgtgttgtttgtttgttctccctttgctacaaaaatcagaatttctactcaataaacagcaaagggagatacaaatgaaccaaattaaagaaggaaaaaatgttgaaaaaattatatacagaactatgtattgatttattgagagttcagtaatgtaatccagaaataatggatgccttaaaagtaattaaaagaatgcaaataaacatttagtgccaattaaagaaaaagaaatacaacattagacaaaataaaagatattcatttgatgcaatgaggaaataatcttttattcctctttaaattctctgtggaataaggcatggttataaataaataaacatctgccccatggacttaatggatcgttatattttattgcgataatcataatgaaattgttgggagggattagtatctctagtgtaatgctaagaaagataaagcctgtgcccaggcaaaagctttcttggttggtcaaaaggtttgaagacatttcaaactattctaaaacaaacaaacaagcaaacaaacaaaaaacatacaatgtctttgccacatatttaggaaacaaaatgaacaatttatttctgacaacctcatagtctttgttctgtcagaacaataatggaaaggtctaaaccagaaaatgctatgcattgaatttataataaactattttttcctgtaacaaaaaattgataaacttgatatttgcagatttaatgattatgtgtttaaaaaaaatctggtttttgcccttgcaaaaaatcatatatatacacatagatatgtatgtgtgtgtgtgcatagtatatatatatgtatatacatatatatacacacatttatatatataaacatttcctttaacctcctattttattccaataaaaatattggtattagagatagttctgatatttcatcatgaatagttaacattgcatttggaaaggattaatttttttgaaacgtaattttaccttaataagtagcccagcgtaatattttagtaattacacagatttttttttcaagacatttgacaactaatattgcataatagttaagagtgtgggctttggagccagacttcctatctctgttcattcactgataaaatggagacagtagtaacttcctcaaagagttgttttttaagatcaaataatgcatataaaactcttgaaatggtaccaaatacagagtaagcaccaaataaacattaactgttattgttattccatgtccgaataacacagaaaagtaagaattttaatatttcatttgaatgaccttttaaggatacacctagcccattatctttcttgataatcttgtaagatgattccttttttatctccgatctgttgaggcatggatagaggttttcagagaaaacattttctaggtaactgaaagaaagtagcaacaacaaactgtgacaaaacttaacaatgagagaatttacaagatagaataattgcaactccttttgaaatcaaccactatggtcctctggctgggatagctaagcaaagatattccagcctgaaggttgagatctacttgaagagttttctatccagattgtgagggcccctcaaacttcacttagtatctgtttctattagtatggaaacttctggaaccttgtggtatcacattcacttgactactttattcctgctctagctatcttaaagcctttcttaatcttttatcttttagagaagatacttctaggttttaaatccaccgatcttgaagctattgccttcactctctgcttcagagcccatccttttgtatatgagtagtttgttttgcctaaagtactttctcccagtcagattttaagtccagtttctcatctgtttttgagagcaaactcctgggccttggctcactaacatcttgacagcatatttcttctttcctatgggcttttcagcattccctgggtttttctaaaatatgaaagcagactctttatctcttactttgtcaaagcctaccctccccactgatttctcacccagttgctagttttaagacctgcctctggccgggcgcagtggctcacgcctgtaatcccagcactttgggaggccaaggtaggtggatcacgaggtcaggagatcgagaccatcctggctaacacagtgaaaccctgtctctactaaaattacaaaaaaattagccaggcgtggtggtgagcgcctgtagtcccagctactcgggaggctgaagcaggagaatggcgtgatcccgtgaggcagagcttgcagtgagctgagatcgcgccactgcactccagcctgggcgacagagcgagactctgtctcaaaaaaaaaaaaaaaaaaaaaaaaaaaaaaagacctgcctccaaatatcattgtatttgcaaacatgaaatgacttattgattctgagctcagcacaagagcaaacctttctcagcttgacccatcttcacatcgttaatgtcttattcagtcactacccaaggggctgaccttcaagattctaatccatgaaagcttaaaatagtaaacaaatttgaatatagtttaacatacataataaattttatttctagaagaggaggatcagcccttagacatgaaaagtaaaaatagtttattcccagatttccctttgtgcattagtatattcaaccgagtctatccaagtaacaggacaaaaaaagctggcagttgttgctgcgctgtgaagtcttattaggtgagtcagctaattatatggcactaccataaatacagcaggcactgccctgcttgttaggcttgccaaggaaaataaggatttaaagcagcatactacctctttgctatataatgacattttcttcttaaaaatgattttgcaccaattcctgatttatccaccaattattttttaatttatggttgaatgtatttaaacctgaattcagagataaaactagtaaatagctccccaaaataaccccaaatatatttaatatattagctttactctctcctccactgccaaacctttaaaaactgaaataaattgtttttatttcatcttttctctttttctctctctctaaggtgattgccaagactaaagaaacagctagaagggcaaaagacaagaaaatcagtaagatagtaacagattatccaaagtagagcacggctcaggtgcagtggctcatgcctgtaatcccagcactttcggaggctgacgcaggaggatcacttgagtccaggagtttgagaccagcctgggcaacataatgaaacttcatctctataaaaaaaaaaaatttaaatagccgagcatggtggtgtaagcctatagtcccagctatttgggaggctgaggctggaggatcacttgggcccaggagttggagactacagtgagctatgattgtatcactgcattacagcctgggcaatagggcaagaccctgcctctaaacaaaagataaacaaagtagagcataaatggcttctaaatatatgttatttatgtgtaagactgggttctctaaaggtatcatttaattaaaatagatttgcattctcaatctgtaggtatggattatgtataatgtatttaagatatgacttacagcgttcaccaatgtgactattcccaagtgatccagatggctgatgacatagtaatttgtacatttgctgagacctgatctgagtaggtatgtaacataactgagggagagcaagtccatttgccgaaagaaagcctagcatatgacccaggagccacatcttcactcagccttgttgctaggtttggcttagcatatataatagcatagcatgtataatttatgacaaaaaattatactttgcactttttaattagaacattcaaaatgatctcaggaagtggcaccagagatcatcagtggtctactgtacttcgtgtgtatgtgtctgtgagtatgtatgtgtttgtgtgtgttcccacattctaaggcatgtcttttacaggttagtagaaaatgttgatagaaaattatagatttcaacatctaaaacacagtaggtcactacattgttaaaacttggaattttttatcttgttgtaaagtcaggccaaccaaacctaaaatactgctacattgaaatagtgcaaaatattcaaaatactatagttatagatttggtagtaggactgtaccagacctgtcactctatacaagacttatgccttgccctttcacttacctgttcccttttacatctatcttactagatgtaatgctataaattatatttctaatatattataatttatcatgtattataatgtatcaaatattacaaattatgttgcaactccccttacctttcgtctgcatattgcctcagaaagaacagatggatccaacagacttcaaccacaggcccttagtgacaaatagctcttaatgctgggcttgccactttgatgcatttctaaagttatagaatgttaaatgcaccaagtcctttggtcattttatttctaccttagatctaagccataactatactttcccaaaaattaaagtttgaattttaacttaaccatatataattggaaaaggaggttgggttcgttaagtgtaattttatcatgctttattatcctttgggcattggatacagcagaacatgccaatttctatggcttctcatgtgacagaatatacttactaggatgcaattaaatactcctcagagtatgtaaacaataaatgtaatcattacattatttttatattgttctttcttatgcataatagtaagactgaaaatatagtgttatttctgaaatatgcatattgttttgcttttgatgattaaataacattgtccaaagttttaggttttttgaaatcttatattttttaacaaaatatctagcctttccaaaacaagacctcaataattcgtttaagacccagagttgttcctctccacatagatctcttaaaaaggcagaggatttatgacctcaagagaaatcagagtatccaaagtttgctttaattcaatgttttaaaaataaaattccttagattttatcaaaaattgagattagtttgattttgaatcagatgccctttgctccccaccccaaaatggcattatgagcagactaggaattgataatagaaaattgaacatatgaaatatatctttaccttgctttttaacaaggtattcatgtctatcgccttcatttttaagtgcatcaataaaatacatggtaattctcttagtgaaatatactatctacactatgtacacactcccctgtctgaggtagagaagtagagaatattcacatttttgaaacgtctatgctatttttatttaaatacgagttctgggcttgatttcattttggaacacgggtgtgtgcttaagttgaacctttttttcctcttaagtcaaagttcttttttagtttcttcttttatctttttggctactatctctctccttcatcctcctggtgtgagttgttgagtgaaggtattaattccattatttgaggctaagtgacattgttcaataatgcagcaaaacaatggttctacccaaaatatcttcaagtgtaaaagcagtgggcaaaagagaaagtgcgcttctgctgctttgaatgtttaaggctgtgaaagttgatcacacaaattgggtcattcttgttatacccaactaaaacaatcaagaagcctgggaggaaaagcattcaagaaacatcacattgctccaaaagtgtaattttctacaagtccgcatgctgaggctgcctgttgtaacctgggaccaattttttctgtaactgctgaaaaaacttgctgcagctctaggactaattttgcccaccactgtcactcaccaattgaagcttactagctccccagaacctttctagtgccaatgaactttctcaaagagcagcgtgtatcatttctctttttcagaacacctccaacctcctctttgttctttgggtataccaaagaccaaccagccttgaatttcaatttttcttcccacataaaagttttaatttagaaatgtatctctacatttctaactttgacaaagcatagataccagataattgatgaaaccttgctattttaacgatcaccatggattacttcccagtgtcttcagataaccctcaacatttgccaacatttgatggacttcaaaatgagcatatcttttttaaaaaaaattattcacactgacagcaagtacattggtatactctatattaaattataccacagggtttacaaacaattggtgatgtcgggcagtggtttccaaggaacatacttaacaagacactcacaaggccctacaaacctgcatttttaacaagggccctagatgattctagaagagtgtggtttggaaagcaatttttgcctttattatgtgtcattttaaatatatttaaaattaaagttataagtcatagaattgaataaagataatttccttacagaaagtattactaggtatctaaatacaatatggttcaaaacaggaaatttaaaaagattatgtaaattctgtagttgtattcctaaagacagtagctgaaattttttcctacttctccttgtatcacttcccttttccttcactttcacttccctggaattgtacttcccaataagctattagcagtgaaggaagcttcgtctcatgatctgttttatagagcacttcagctgggacgagtacgaaatgataatcagttatatcagctattcaaccctacaggtttatttaaaaagaacttgaataagctttttagggagaaagaggtcagtctcagccatttctgtttcctaatatagcttttaagtctttccttattagcaatgagggtcattccattgtaattttttgataaccatttttctttctgtgtgtcaaatgcagatataagatactgaactgagtctatttcactgttcgtaaaacaatcccatttgaaaaaaaaaagtctacagctattccagggatagggcctagtagagagagaataaaaggtattttcttactatgtctctatatcctaccctgtaggttctcttattaagcatacaggcatataccaaaatccagacgtttttctcatttattttattgccctaacatattctgggttaatataatatcataatgaaaatttgagaaaaaattgattttttcaaaagtgtttaacatttgttatattggtagttttttttcttgtttgtggtaaaaataaatagaaggtgcacttcacaccttcaagtatgattatattttgaaaacaagtcatgaatactcataaaatgcaaattttaatgttctttttttgttacagccaaactatattaggcacagttgtaaattggagttgaaatttaatatttctttatagataacaatgtttttagaaataggtttatgaaacagtaaatatacaggtatagggataaaattgtgtctgatggtcatatgaagtgtttgttgttatattctccttggaatagctgccaaatattttagtatgcttaaaatctacgaatgtgatagagtcaacaaatttagatcacatattcagaaaaacatagttagagaactaactattgaaatgagcatacagcagtcttcctttatctacagggatacattctgaaacccccactaggacacctgaaattgcggatagtagcaaaccctacatatactgttttttccaatgcttatgtacctatgaaaaagtttaatttataaactaggcacagtaagagattaacaacaataactaataacaaaagagaacaattataataatatactgtaataaaagttatgtgggtatggtctcgctttctctttccctctctctctgtctctaaatatcttagtattttggggttgcaattggtggtgggcaactgaaaccatggaaaacaaaaccacggataaaaggagactactgtatatactttttaaaactgatgaaatattaaactcatgtttcttctatatcccacccatttcccccacccaaacctagatagatatcttatttgatctgtaaacatttaattaatttgtaaaagttaagaactttttgaagtaaaactgcaatatatcatcacacctaaagaaataaacaataattcttaaatatcaagtcagtgttcaaatttccccaactacctcatatgtgttttccatttgcttatgtagggttcccaatgagaatgaaataaagttcttaggttgcaattggctaatgctctctcacttctactttaagcggcaggttcccactaacttctttttagttgcaatttacttattgaaattagacgtattctttgtcttgtgtagtttctcacagtgcaaaatttgctgattgtagccactgttgtaagcaatgaacatgtttttcaccaccttatatttgctgtaagttgtcagtgatagttaaatgttaatcaaattcaaattcggatcacgtagggcttttctttttttgttttctttttctatttatatatttatttatttattttgagacggagtctcactccgtcaccaggctggagtgcaatggtgtgatctgggctcactgcaatctccacctcccgggttcaagtgattcccctggctcagtctcccgagtagctgggactataggagaaccaccacgcccggctaactttttgtattttagtagagatggggtttcaccatgttggccaggatgctatagatctcctgacctcaccgatcatgtaggacttcaattgtcgaacaaacgaacctttaatagcagttacaccattaggatgacctgatccaacatcgaggtcgtaaaccctattgtcgatttggactctagaataggattgtgctgtcatccctagtgtagcttgttcccacttgatgaagttattggatcagtgaacaatagcccacttaaactagtacagtcttagtttaagatggtgatgtgtatgtacttccatcagagggcacataatacagtaaatcctcacttaacttcatcaatagtttctggaaactgtgacttgaagcaaaacaacatataacaaaaccagttttaccattggctaattgatataagcaagaattaagtcctatggcaaatttctggacacaaaaacaccatcaaactcctaaataaagataaatcacttctgacattaaacattgaaattaatgtgagctatatatacgtttaagaaagattaatacaaacaagtcaaataacttacctaattatttcggtggaggccgcaggtggttggagcctatcctggcagctcagggagcaatatgggaacccaccccggacaggacgctgttccattactgcagggtgctcttgtacacacccactcacccaggctggaaccatgcagacacacacactcacctaacctacacatctgtgtacatccttcaaagttcagccaaataacatataaacaaatccagtaatatccatcagtcttagttccgtcataacaactcctttttgatcatcaaacaacaaacagggtaggtctgccatatttacttgtctggtccatatcaaaattttctaacaaattatattagaaaatcaaatctctgtcagtttcaaaatcatggaaaaaaatttgccttatttcccttatacttggatatcctaacagtaatctaaatattaatgagaaagttaatgatgtcgtttccttctccctgttgtaaagaaggttttgctgtcccgtttgatcactaagactaattgacactcagaaaaagcataggaaacttctcagcatcacaaaagctctgtcatctagagaagctaggacttgagctcaagtcctgtgacatggaaggccttgtgcctagccatcctgcagcagaggcgtatctaccaagaagtgaaacactacgaaaacagtatgtttactccacattttaaagtgaggtagtttggggtggttcatattttatttaatttatatattatttggattttttttagtttataaaaagggcattggcaagggcagaatgatctgtaagcttctctgcccacctaccataagcatgatctttagtgtgaccttttcttactgttagccattttcttatacttctgcgtccctgtcagtcacttccatgtgaagacatggggaagcttttttacatcagacatgttgttgaaaatcagccgcgttggctgagggattatttgatctctttctccaagtccctttaggctcacattgcctctctgttctttgaattttcacttacctttatcttcttataattactttgctgaaataaatgcaaagcaacaaaaggtatttagtgaagaataccaacaaagccatgaccatttcaggctgagttttgtagtattctttgtctaggaagagatacctagaaaaattttctgaccatgtatttgattattttccttcaatatgtatagtctcagtcttcaaatttcagaaaagaatttgtttcttcattgtcatttaaaattaatgtgttaaatatgtatgcttttacattataagtggttataaaagttaaacacttagaaaaaaagtcaaaataacatacatactatccaacaaaataactttcatattttattgtgttttcttccaaactttttacctttgcgtctgaattctgtgtaggttgtatctataatatagacaacactttatagcctgctaaatattataccataaataggtagttgttacataattctcaggtaatagtaatacaggtctttatcataatctactgagtagttgaatgataattttttttaagacaaggtctccctctgtcacccaggctagaatgcagtggcatgcacatggctcactgtagcctctacctcccaggctcaagtgatcctcctgcctcagcctcccaagtggctgggactgtaggcatgtgccaccatgcccagctatttatttgtatttttagtagagatggggtttcattgtaacagcccaggctggtcttgaactcctggactcaaatgatccacctgcctcagcctcccaaagtgctgaaatcacaggagtgaaccactgcacccagcaataattttttaactcttcattattcattgaacatttagttaacaattctaaaaattttgtttcctgctgtcattgatcttgtgaaaaatatctttggactatagctgtggattatttcctaaatagtaaattacttgagcaaaaagtttacatactttgagggttgataacccatgttgccgcaatgtttccccggaggcattgtggagtttagaatgccagtagtaatattaaggtgtgccattttcaagatccgtggccaacatccctatatgtaagatttttccaaaacatggttctgatttttaaaagtgaaaaatgctacttcatcatgttctttttgtgcttcttactttaaatattagaatgaagaaggagccccacaggaaggaattctggaagatatgcctgtggatcctgacaatgaggcttatgaaatgccttctgaggtaggagtccaagctgaatctttctaacaagacagtaccaaaaacctgtcattgtcacatttctctttcattagtgcttagtgagaatcatttgctctctacatgctcattacgtggacaacttgcaagttaagaatagtttttacatttttaaagggtccttaaaaaaaaagaggaggaggaagatgaagaagaggaagaaaggatgtaaaagaaatcatatgtagtccacatagcttaatatacttactacttgaccctttacaggaaaagtttactaacccctgcattagagaatatatttttagaaactttacattctaaaataaatttctaaatggaaagttagggaaatcaatggaatgccaaaggaaggttattattttttgccatacatgtccaatgggatgacgcatagtaaaataaaagttacccacacaagttatagaataaaaagataaatgcatgatttgcgacaattgatatattccagtataatgttttaaacaacacaatatgattgttaattttattttgattgaaaatgaaagtatctttaatagaaaatgtatcaaaagggaaattagaaaatactgttagatgaataaaactggcccaagaagaaacagtaaatctgaatagatttgtaacacagcgaatagattaaattagtaataaaaaaaaaaacctacctgcaaagaaaatcccaggccgagatggcatcactggtaaattctaccaaacatttaaagaggaattaatactaattagttaacaccaattaatatctcttacaaaacagaagaggagacatttcccaactaattttgtgagaccaatattaccctgataatcaaaaccaaatgaagatatcacaagaaaagaaactatataatggctccattaaaaattgagttcaagtatgttgtagtttggttatgtattattcctcacggcattattaaaaggcatgtcgaggatgggcacagcagttcacacctgtaatcccgcactttgtgagccaaagtggccaggttacttgaggccaggagttggagaccagtctggccaacatggtgaaaccccatctctactaaaaatacaaaaattagccgggcatggtggtacacgcctatggttccagctacttgggaggctgaggcatgagagtcacttgaacccaggaggcagaggttgcagtgagctgagatggcacccctgcactccaatcttggtaacagagcaagactgtctcacacagacacacgaaaggcatattgataataattcaacttatagaaattgagattaaattgtttgtttgcctaataagaatttccaatattttggggtcttttatgcaagacacagtactaaacacaatggaaaactatagagtaattgacattaccaggacataaggagtttacagtctggtaggtttgatgaaaaaaaatagaaattcattcattcatttcttcattatgattcctttaacaaacataattgattgtcttcgatgtaccaggcatcacaggagcaaaaatatataagacatactaaaaagtaaaacattttaaagatctgtttcaatcaatcaggagaagttttattgaggaggtaatgttgatctgggtgggaaaaggtaagagatatagtaggtcaaaacaaacagaggacattctggcacaagggaatatcagaagcaaaggcatgtatgtctgagcatgcaaatggatatgtctgagaacagtgaataattatgactcaagcttaggaacaaggaaaatggtgatagattgaatttgcagctatgggtcaaagacaagttatagagtattaggataatcttgtcatttcagcttgtattctattcagaaaacaacttgagttattgaagttatgcttatttgtttgtttttaagcagaatcctgatattattagagttgctctttaggaggaataatctgatccctttaattaaatccattaatatttgtgttgtggatgctatccagatactgtatggagagcttgaggtttgaaatacaagtaataattgaagccatagatgaagacgaaattttcaactgggagagtgaaagtagggaaaatgtatcttgccttcaaacatcttaatttccttctgagaattagagcatcttagtctggaaaaggctttatagacagcttgattttgttctcacattttacaggtgaagaaactgagaaccagacagtccaacttatttgtcctaccaaactaggtatatgatcattaaatggtgcatccggatcagaacctagatattttaactctgactactactgtaattcacttttatatcagacaagaaagacacaactattaaaaataagataatatttgctgcagaatatttgcaaaaacattgattgtaaattttagtgtaagtggggagccatttcctatctcattggctgtcagtgctgatgcgtaattgaaacttatactaacagtgtgtgctgtctttttgatttttctaatattaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaatatgtatttggtttctgcctccagttcttacaaagagctcctaaaacccttgtaatttcctgagtagtaggggtgctagggtcatcttttgttctaatatttggtctttgactctgctttctgacagagctccttagtccctgggtgagagtagcatcttctcttctaatgaagtgactcttgctgggttcctggatgggggctggtcaccagaaaggtcaagccatgataagaagcttgaagcttttggccccattcacatcttctggggacgggagagaagaggagctggagattgagttaataagcaacaatgcttccatgatgaagactccataaaaatccctaaaagacaggattcagagtgctttgaaataggtgaacatgcagaggtgctgggaattgtggtgtgtccagagaaggcatgcaagctccccacgcctcccccatacctttccctgtgcatctcttccatctggctgttcctgagttgtatccttttataacaaactggtaatctagtaagcaaactgttttcctgaagtctgtgaatcacactagcaaattatcaaacctgaggagagggccgtggagaccttggatttgtagacaagtcaaacagaagctatgagtaacatgaggactcattgcttgtgattgtcatcttcagtgggaaggggaaaaatcttgtaaaactgagtccttaacctgtgggtcaatgctaactccaggtagatagtgtccgatttgaattacgggacacccagttggtagccacaaagaatgggagaattgcttggtgtagaaaacacaccccacacacacatgtggtgtcagaaatgaaccggaaatattgtgttccggaaatattgagtgttgtgagtgagtgtatagaaagaaaaacagcgtttccttttcactactagattaaaacaaacacactcatgcattcacacatctcaaagacaactattaattctcaaagacagtgctgtctaaatccatactgaggaagaaaacacattttcttttcaaatctgtaaacctgacagactgcctctgtccacacactaatggaactctgtgtttcatctgaaatgtgttcatcccactttgttctttctgtcttgggcagggcaagagtgcaacagggctgacattttcatatgagctctgtccctgttattggctatactttagacaaattattatgtgtcaaatatagatgtaagtgatttatcaatattaagtcatttaattctcaaaacaaccttaataggttccattatgattctaattttacacataagccaaaggaggcacccacaggctagataactttcccacggccacacagctagtaagcggcagagccaagaggcccaacattacagcaccacagtctgtgctctcagccccttggccacatagtgtcagagtgaggacacacagctatttaagaaaacttccagaagtctaggaaatggggtgatagccccacttttctaggtataataattagatatttgtttttcttcaggtacctaaagaaaatttactagagtttgagcctttagtaagttttgctagtacatctgtttttcttcaggtgcctgaagacaaacatatacacacacacacacacacaaacacacacaaaatgtgtatctatatatatgtgtacacatatctctcatctctatatatatgtctctgtatatctatatatctataaacatatctatatctatagatacatatagagagatttctttttttttttttttgagatggagtcttgctcttgccacctaggctggagtgcaatggcacaatctcagttcactgcaacctccgcctcccaggttcaagcgattctcctgcctcagcctctcgagtaggtgggattacaggaacacaccaccttagcccgactaatttttgtatttttagtagagacagggttcaccacgttggccaggctggtctcaaactcctgacc

SEQ ID NO: 2 - RNA, 3211 nt, Homo sapiens
gccauucgacgacagguuagcggguuugccucccacucccccagccucgcgucgccggcucacagcggccuccucuggggacagucccccccgggugccgccuccgcccuuccugugcgcuccuuuuccuucuucuuuccuauuaaauauuauuugggaauuguuuaaauuuuuuuuuuaaaaaaagagagaggcggggaggagucggaguuguggagaagcagagggacucagugugguguaaaggaauucauuagccauggauguauucaugaaaggacuuucaaaggccaaggagggaguuguggcugcugcugagaaaaccaaacaggguguggcagaagcagcaggaaagacaaaagaggguguucucuauguaggcuccaaaaccaaggagggaguggugcaugguguggcaacaguggcugagaagaccaaagagcaagugacaaauguuggaggagcaguggugacgggugugacagcaguagcccagaagacaguggagggagcagggagcauugcagcagccacuggcuuugucaaaaaggaccaguugggcaagaaugaagaaggagccccacaggaaggaauucuggaagauaugccuguggauccugacaaugaggcuuaugaaaugccuucugaggaaggguaucaagacuacgaaccugaagccuaagaaauaucuuugcucccaguuucuugagaucugcugacagauguuccauccuguacaagugcucaguuccaaugugcccagucaugacauuucucaaaguuuuuacaguguaucucgaagucuuccaucagcagugauugaaguaucuguaccugcccccacucagcauuucggugcuucccuuucacugaagugaauacaugguagcagggucuuugugugcuguggauuuuguggcuucaaucuacgauguuaaaacaaauuaaaaacaccuaagugacuaccacuuauuucuaaauccucacuauuuuuuuguugcuguuguucagaaguuguuagugauuugcuaucauauauuauaagauuuuuaggugucuuuuaaugauacugucuaagaauaaugacguauugugaaauuuguuaauauauauaauacuuaaaaauaugugagcaugaaacuaugcaccuauaaauacuaaauaugaaauuuuaccauuuugcgauguguuuuauucacuuguguuuguauauaaauggugagaauuaaaauaaaacguuaucucauugcaaaaauauuuuauuuuuaucccaucucacuuuaauaauaaaaaucaugcuuauaagcaacaugaauuaagaacugacacaaaggacaaaaauauaaaguuauuaauagccauuugaagaaggaggaauuuuagaagagguagagaaaauggaacauuaacccuacacucggaauucccugaagcaacacugccagaaguguguuuugguaugcacugguuccuuaaguggcugugauuaauuauugaaagugggguguugaagaccccaacuacuauuguagaguggucuauuucucccuucaauccugucaauguuugcuuuacguauuuuggggaacuguuguuugauguguauguguuuauaauuguuauacauuuuuaauugagccuuuuauuaacauauauuguuauuuuugucucgaaauaauuuuuuaguuaaaaucuauuuugucugauauuggugugaaugcuguaccuuucugacaauaaauaauauucgaccaugaauaaaaaaaaaaaaaaaguggguucccgggaacuaagcaguguagaagaugauuuugacuacacccuccuuagagagccauaagacacauuagcacauauuagcacauucaaggcucugagagaaugugguuaacuuuguuuaacucagcauuccucacuuuuuuuuuuuaaucaucagaaauucucucucucucucucucuuuuucucucgcucucuuuuuuuuuuuuuuuuuacaggaaaugccuuuaaacaucguuggaacuaccagagucaccuuaaaggagaucaauucucuagacugauaaaaauuucauggccuccuuuaaauguugccaaauauaugaauucuaggauuuuuccuuaggaaagguuuuucucuuucagggaagaucuauuaacuccccaugggugcugaaaauaaacuugauggugaaaaacucuguauaaauuaauuuaaaaauuauuugguuucucuuuuuaauuauucuggggcauagucauuucuaaaagucacuaguagaaaguauaauuucaagacagaauauucuagacaugcuagcaguuuauauguauucaugaguaaugugauauauauugggcgcuggugaggaaggaaggaggaaugagugacuauaaggaugguuaccauagaaacuuccuuuuuuaccuaauugaagagagacuacuacagagugcuaagcugcaugugucaucuuacacuagagagaaaugguaaguuucuuguuuuauuuaaguuauguuuaagcaaggaaaggauuuguuauugaacaguauauuucaggaagguuagaaaguggcgguuaggauauauuuuaaaucuaccuaaagcagcauauuuuaaaaauuuaaaaguauugguauuaaauuaagaaauagaggacagaacuagacugauagcagugaccuagaacaauuugagauuaggaaaguugugaccaugaauuuaaggauuuauguggauacaaauucuccuuuaaaguguuucuucccuuaauauuuaucugacgguaauuuuugagcagugaauuacuuuauauaucuuaauaguuuauuugggaccaaacacuuaaacaaaaaguucuuuaagucauauaagccuuuucaggaagcuugucucauauucacucccgagacauucaccugccaaguggccugaggaucaauccaguccuagguuuauuuugcagacuuacauucucccaaguuauucagccucauaugacuccacggucggcuuuaccaaaacaguucagagugcacuuuggcacacaauugggaacagaacaaucuaaugugugguuugguauuccaaguggggucuuuuucagaaucucugcacuagugugagaugcaaacauguuuccucaucuuucuggcuuauccaguauguagcuauuugugacauaauaaauauauacauauaugaaaaua

SEQ ID NO: 3 - RNA, 3022 nt, Homo sapiens
auucuggugugauccaggaacagcugucuuccagcucugaaagagugugguguaaaggaauucauuagccauggauguauucaugaaaggacuuucaaaggccaaggagggaguuguggcugcugcugagaaaaccaaacaggguguggcagaagcagcaggaaagacaaaagaggguguucucuauguaggcuccaaaaccaaggagggaguggugcaugguguggcaacaguggcugagaagaccaaagagcaagugacaaauguuggaggagcaguggugacgggugugacagcaguagcccagaagacaguggagggagcagggagcauugcagcagccacuggcuuugucaaaaaggaccaguugggcaagaaugaagaaggagccccacaggaaggaauucuggaagauaugccuguggauccugacaaugaggcuuaugaaaugccuucugaggaaggguaucaagacuacgaaccugaagccuaagaaauaucuuugcucccaguuucuugagaucugcugacagauguuccauccuguacaagugcucaguuccaaugugcccagucaugacauuucucaaaguuuuuacaguguaucucgaagucuuccaucagcagugauugaaguaucuguaccugcccccacucagcauuucggugcuucccuuucacugaagugaauacaugguagcagggucuuugugugcuguggauuuuguggcuucaaucuacgauguuaaaacaaauuaaaaacaccuaagugacuaccacuuauuucuaaauccucacuauuuuuuuguugcuguuguucagaaguuguuagugauuugcuaucauauauuauaagauuuuuaggugucuuuuaaugauacugucuaagaauaaugacguauugugaaauuuguuaauauauauaauacuuaaaaauaugugagcaugaaacuaugcaccuauaaauacuaaauaugaaauuuuaccauuuugcgauguguuuuauucacuuguguuuguauauaaauggugagaauuaaaauaaaacguuaucucauugcaaaaauauuuuauuuuuaucccaucucacuuuaauaauaaaaaucaugcuuauaagcaacaugaauuaagaacugacacaaaggacaaaaauauaaaguuauuaauagccauuugaagaaggaggaauuuuagaagagguagagaaaauggaacauuaacccuacacucggaauucccugaagcaacacugccagaaguguguuuugguaugcacugguuccuuaaguggcugugauuaauuauugaaagugggguguugaagaccccaacuacuauuguagaguggucuauuucucccuucaauccugucaauguuugcuuuacguauuuuggggaacuguuguuugauguguauguguuuauaauuguuauacauuuuuaauugagccuuuuauuaacauauauuguuauuuuugucucgaaauaauuuuuuaguuaaaaucuauuuugucugauauuggugugaaugcuguaccuuucugacaauaaauaauauucgaccaugaauaaaaaaaaaaaaaaaguggguucccgggaacuaagcaguguagaagaugauuuugacuacacccuccuuagagagccauaagacacauuagcacauauuagcacauucaaggcucugagagaaugugguuaacuuuguuuaacucagcauuccucacuuuuuuuuuuuaaucaucagaaauucucucucucucucucucuuuuucucucgcucucuuuuuuuuuuuuuuuuuacaggaaaugccuuuaaacaucguuggaacuaccagagucaccuuaaaggagaucaauucucuagacugauaaaaauuucauggccuccuuuaaauguugccaaauauaugaauucuaggauuuuuccuuaggaaagguuuuucucuuucagggaagaucuauuaacuccccaugggugcugaaaauaaacuugauggugaaaaacucuguauaaauuaauuuaaaaauuauuugguuucucuuuuuaauuauucuggggcauagucauuucuaaaagucacuaguagaaaguauaauuucaagacagaauauucuagacaugcuagcaguuuauauguauucaugaguaaugugauauauauugggcgcuggugaggaaggaaggaggaaugagugacuauaaggaugguuaccauagaaacuuccuuuuuuaccuaauugaagagagacuacuacagagugcuaagcugcaugugucaucuuacacuagagagaaaugguaaguuucuuguuuuauuuaaguuauguuuaagcaaggaaaggauuuguuauugaacaguauauuucaggaagguuagaaaguggcgguuaggauauauuuuaaaucuaccuaaagcagcauauuuuaaaaauuuaaaaguauugguauuaaauuaagaaauagaggacagaacuagacugauagcagugaccuagaacaauuugagauuaggaaaguugugaccaugaauuuaaggauuuauguggauacaaauucuccuuuaaaguguuucuucccuuaauauuuaucugacgguaauuuuugagcagugaauuacuuuauauaucuuaauaguuuauuugggaccaaacacuuaaacaaaaaguucuuuaagucauauaagccuuuucaggaagcuugucucauauucacucccgagacauucaccugccaaguggccugaggaucaauccaguccuagguuuauuuugcagacuuacauucucccaaguuauucagccucauaugacuccacggucggcuuuaccaaaacaguucagagugcacuuuggcacacaauugggaacagaacaaucuaaugugugguuugguauuccaaguggggucuuuuucagaaucucugcacuagugugagaugcaaacauguuuccucaucuuucuggcuuauccaguauguagcuauuugugacauaauaaauauauacauauaugaaaaua

SEQ ID NO: 4 - RNA, 3215 nt, Homo sapiens
aggagaaggagaaggaggaggacuaggaggaggaggacggcgacgaccagaaggggcccaagagagggggcgagcgaccgagcgccgcgacgcggaagugaggugcgugcgggcugcagcgcagaccccggcccggccccuccgagagcguccugggcgcucccucacgccuugccuucaagccuucugccuuuccacccucgugagcggagaacugggaguggccauucgacgacagugugguguaaaggaauucauuagccauggauguauucaugaaaggacuuucaaaggccaaggagggaguuguggcugcugcugagaaaaccaaacaggguguggcagaagcagcaggaaagacaaaagaggguguucucuauguaggcuccaaaaccaaggagggaguggugcaugguguggcaacaguggcugagaagaccaaagagcaagugacaaauguuggaggagcaguggugacgggugugacagcaguagcccagaagacaguggagggagcagggagcauugcagcagccacuggcuuugucaaaaaggaccaguugggcaagaaugaagaaggagccccacaggaaggaauucuggaagauaugccuguggauccugacaaugaggcuuaugaaaugccuucugaggaaggguaucaagacuacgaaccugaagccuaagaaauaucuuugcucccaguuucuugagaucugcugacagauguuccauccuguacaagugcucaguuccaaugugcccagucaugacauuucucaaaguuuuuacaguguaucucgaagucuuccaucagcagugauugaaguaucuguaccugcccccacucagcauuucggugcuucccuuucacugaagugaauacaugguagcagggucuuugugugcuguggauuuuguggcuucaaucuacgauguuaaaacaaauuaaaaacaccuaagugacuaccacuuauuucuaaauccucacuauuuuuuuguugcuguuguucagaaguuguuagugauuugcuaucauauauuauaagauuuuuaggugucuuuuaaugauacugucuaagaauaaugacguauugugaaauuuguuaauauauauaauacuuaaaaauaugugagcaugaaacuaugcaccuauaaauacuaaauaugaaauuuuaccauuuugcgauguguuuuauucacuuguguuuguauauaaauggugagaauuaaaauaaaacguuaucucauugcaaaaauauuuuauuuuuaucccaucucacuuuaauaauaaaaaucaugcuuauaagcaacaugaauuaagaacugacacaaaggacaaaaauauaaaguuauuaauagccauuugaagaaggaggaauuuuagaagagguagagaaaauggaacauuaacccuacacucggaauucccugaagcaacacugccagaaguguguuuugguaugcacugguuccuuaaguggcugugauuaauuauugaaagugggguguugaagaccccaacuacuauuguagaguggucuauuucucccuucaauccugucaauguuugcuuuacguauuuuggggaacuguuguuugauguguauguguuuauaauuguuauacauuuuuaauugagccuuuuauuaacauauauuguuauuuuugucucgaaauaauuuuuuaguuaaaaucuauuuugucugauauuggugugaaugcuguaccuuucugacaauaaauaauauucgaccaugaauaaaaaaaaaaaaaaaguggguucccgggaacuaagcaguguagaagaugauuuugacuacacccuccuuagagagccauaagacacauuagcacauauuagcacauucaaggcucugagagaaugugguuaacuuuguuuaacucagcauuccucacuuuuuuuuuuuaaucaucagaaauucucucucucucucucucuuuuucucucgcucucuuuuuuuuuuuuuuuuuacaggaaaugccuuuaaacaucguuggaacuaccagagucaccuuaaaggagaucaauucucuagacugauaaaaauuucauggccuccuuuaaauguugccaaauauaugaauucuaggauuuuuccuuaggaaagguuuuucucuuucagggaagaucuauuaacuccccaugggugcugaaaauaaacuugauggugaaaaacucuguauaaauuaauuuaaaaauuauuugguuucucuuuuuaauuauucuggggcauagucauuucuaaaagucacuaguagaaaguauaauuucaagacagaauauucuagacaugcuagcaguuuauauguauucaugaguaaugugauauauauugggcgcuggugaggaaggaaggaggaaugagugacuauaaggaugguuaccauagaaacuuccuuuuuuaccuaauugaagagagacuacuacagagugcuaagcugcaugugucaucuuacacuagagagaaaugguaaguuucuuguuuuauuuaaguuauguuuaagcaaggaaaggauuuguuauugaacaguauauuucaggaagguuagaaaguggcgguuaggauauauuuuaaaucuaccuaaagcagcauauuuuaaaaauuuaaaaguauugguauuaaauuaagaaauagaggacagaacuagacugauagcagugaccuagaacaauuugagauuaggaaaguugugaccaugaauuuaaggauuuauguggauacaaauucuccuuuaaaguguuucuucccuuaauauuuaucugacgguaauuuuugagcagugaauuacuuuauauaucuuaauaguuuauuugggaccaaacacuuaaacaaaaaguucuuuaagucauauaagccuuuucaggaagcuugucucauauucacucccgagacauucaccugccaaguggccugaggaucaauccaguccuagguuuauuuugcagacuuacauucucccaaguuauucagccucauaugacuccacggucggcuuuaccaaaacaguucagagugcacuuuggcacacaauugggaacagaacaaucuaaugugugguuugguauuccaaguggggucuuuuucagaaucucugcacuagugugagaugcaaacauguuuccucaucuuucuggcuuauccaguauguagcuauuugugacauaauaaauauauacauauaugaaaaua

SEQ ID NO: 5 - RNA, 2895 nt, Homo sapiens
agugugguguaaaggaauucauuagccauggauguauucaugaaaggacuuucaaaggccaaggagggaguuguggcugcugcugagaaaaccaaacaggguguggcagaagcagcaggaaagacaaaagaggguguucucuauguaggcuccaaaaccaaggagggaguggugcaugguguggcaacaguggcugagaagaccaaagagcaagugacaaauguuggaggagcaguggugacgggugugacagcaguagcccagaagacaguggagggagcagggagcauugcagcagccacuggcuuugucaaaaaggaccaguugggcaaggaaggguaucaagacuacgaaccugaagccuaagaaauaucuuugcucccaguuucuugagaucugcugacagauguuccauccuguacaagugcucaguuccaaugugcccagucaugacauuucucaaaguuuuuacaguguaucucgaagucuuccaucagcagugauugaaguaucuguaccugcccccacucagcauuucggugcuucccuuucacugaagugaauacaugguagcagggucuuugugugcuguggauuuuguggcuucaaucuacgauguuaaaacaaauuaaaaacaccuaagugacuaccacuuauuucuaaauccucacuauuuuuuuguugcuguuguucagaaguuguuagugauuugcuaucauauauuauaagauuuuuaggugucuuuuaaugauacugucuaagaauaaugacguauugugaaauuuguuaauauauauaauacuuaaaaauaugugagcaugaaacuaugcaccuauaaauacuaaauaugaaauuuuaccauuuugcgauguguuuuauucacuuguguuuguauauaaauggugagaauuaaaauaaaacguuaucucauugcaaaaauauuuuauuuuuaucccaucucacuuuaauaauaaaaaucaugcuuauaagcaacaugaauuaagaacugacacaaaggacaaaaauauaaaguuauuaauagccauuugaagaaggaggaauuuuagaagagguagagaaaauggaacauuaacccuacacucggaauucccugaagcaacacugccagaaguguguuuugguaugcacugguuccuuaaguggcugugauuaauuauugaaagugggguguugaagaccccaacuacuauuguagaguggucuauuucucccuucaauccugucaauguuugcuuuacguauuuuggggaacuguuguuugauguguauguguuuauaauuguuauacauuuuuaauugagccuuuuauuaacauauauuguuauuuuugucucgaaauaauuuuuuaguuaaaaucuauuuugucugauauuggugugaaugcuguaccuuucugacaauaaauaauauucgaccaugaauaaaaaaaaaaaaaaaguggguucccgggaacuaagcaguguagaagaugauuuugacuacacccuccuuagagagccauaagacacauuagcacauauuagcacauucaaggcucugagagaaugugguuaacuuuguuuaacucagcauuccucacuuuuuuuuuuuaaucaucagaaauucucucucucucucucucuuuuucucucgcucucuuuuuuuuuuuuuuuuuacaggaaaugccuuuaaacaucguuggaacuaccagagucaccuuaaaggagaucaauucucuagacugauaaaaauuucauggccuccuuuaaauguugccaaauauaugaauucuaggauuuuuccuuaggaaagguuuuucucuuucagggaagaucuauuaacuccccaugggugcugaaaauaaacuugauggugaaaaacucuguauaaauuaauuuaaaaauuauuugguuucucuuuuuaauuauucuggggcauagucauuucuaaaagucacuaguagaaaguauaauuucaagacagaauauucuagacaugcuagcaguuuauauguauucaugaguaaugugauauauauugggcgcuggugaggaaggaaggaggaaugagugacuauaaggaugguuaccauagaaacuuccuuuuuuaccuaauugaagagagacuacuacagagugcuaagcugcaugugucaucuuacacuagagagaaaugguaaguuucuuguuuuauuuaaguuauguuuaagcaaggaaaggauuuguuauugaacaguauauuucaggaagguuagaaaguggcgguuaggauauauuuuaaaucuaccuaaagcagcauauuuuaaaaauuuaaaaguauugguauuaaauuaagaaauagaggacagaacuagacugauagcagugaccuagaacaauuugagauuaggaaaguugugaccaugaauuuaaggauuuauguggauacaaauucuccuuuaaaguguuucuucccuuaauauuuaucugacgguaauuuuugagcagugaauuacuuuauauaucuuaauaguuuauuugggaccaaacacuuaaacaaaaaguucuuuaagucauauaagccuuuucaggaagcuugucucauauucacucccgagacauucaccugccaaguggccugaggaucaauccaguccuagguuuauuuugcagacuuacauucucccaaguuauucagccucauaugacuccacggucggcuuuaccaaaacaguucagagugcacuuuggcacacaauugggaacagaacaaucuaaugugugguuugguauuccaaguggggucuuuuucagaaucucugcacuagugugagaugcaaacauguuuccucaucuuucuggcuuauccaguauguagcuauuugugacauaauaaauauauacauauaugaaaaua

SEQ ID NO: 6 - DNA, 21 bp, Artificial Sequence
  Specific primer for transcript SNCAtv2
agtcggagttgtggagaagca

SEQ ID NO: 7 - DNA, 20 bp, Artificial Sequence
  Specific primer for transcript SNCAtv3
atccaggaacagctgtcttc

SEQ ID NO: 8 - DNA, 19 bp, Artificial Sequence
  Generic primer for SNCA transcripts
accactgctcctccaacat

SEQ ID NO: 9 - DNA, 1980 bp, Homo sapiens
ggaggcagaggttgcggtgagccaagatggcgccattgcactccagcctgggtggcaagagtgaactctgtctcaaaaaaaaataaataaataaaataaaacattgtattataataagggtaaaatctatgtggaatgataattaaaaagttatccttatgcattaataaatacagtatgacctttgtatttgcgggttacacatccatggatttaaccaaccacgagtcaaaaacatttgataaataaatacatctatactaaacatatacagacctttttttcttatcattattccttaaacaatataatataataaacatttacacagcactttcattatattaggtatgaattatctagggatgtgcatggcttatgtgaaaatgttatgctattttacattagggacttgagtatgagaggattttggtatccctgagaggtcctagaaccaactccccacaaatactaaggatgactgtagtcatatataattttcatcagaaataattatctgttatataactatatatgtaaatgagaccattaaatgaaatttgtgttattttaaagtgggtcaagaaaagagcataatatctacatatcctcttgaaaactaaagaaaatatatgtaatgtaattatacataatacaatatataaactcattttccattttatatgttatacgttatataaattatatatacattatttgcattttcacataatctgtaaagattttattaaaatctcttttcaggcaaagcgagctaataacaaataataaagaataaataaagaaaataatgctgtactgtgtagttagagaaaatggcttttgtattcgaaattatttttcagttaatgaaacagataaaaattttgattaatacaacttattccatattttacaggaaatattgatgaagcagaatgggagtggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacgtgtttaattaaatttcacaatataaagttctacagttaattatgtgcatattaaaacaatggcctggttcaatttctttctttccttaataaatttaagtttttccccccaaaattatcagtgctctgcttttagtcacgtgtattttcattaccactcgtaaaaaggtatcttttttaaatgaattaaatattgaaacactgtacaccatagtttacaatattatgtttcctaattaaaataagaattgaatgtcaatatgagatattaaaataagcacagaaaatcattggtctctttgttttttatataaaggcaagaaagaatcttaaaatgagaggctcagccactttaaaacaccttataatagaaacagaatgctgctaccaagaaacaaaatataaaataatgtaattatttatgtaatgccttcaaattatattcttcatttgactcttgctctccctcacctcctctatcctcaaatcaccacaaacatgtacatatgtgaattacctttccgtaattcaaaaatttttaaaaagtgggtgcatagaagtctaaagttaggttgtgtaccatctgggacagggcaacccttgcctggacccagaaatattaacatattaataggaaaattgttcaattccagaagagaaatggttgtactttctgtagacaaactgtcttatttttgcaattaaaaaaattacagagctgcatttattagcatgcttggcaagggag

SEQ ID NO: 10 - DNA, 5040 bp, Homo sapiens
tccatttttatgttactatttccaggaaaaatttaaaatagccttcatatacaataaaagcaagttagataaattatattgtacttatacaaataattaatacataggcatataaatgaagttaaaaaaatattcgatagcattattttggaaaaacttaagataggttatatgaaagtaggctaccaaagactatgtatgatgtatttccaaattagcttgaagcaaaaggtacagttgtgcatgagaataagcaattaaaaaaaagaaaaagcatatatatatatatggtagcacaaaaatgtccttagtatccaagctccaggactttgcctcttagtgtgtagtttgtgaactccctctatcagcttcacagaggacagtagtataacgctgtcctcaagtctactgaaaccaaagcccagggatgtggcctagaaatctggatgtttaaatagcactttgatgaatttgataatgtgttactttgcaatttttctgagagtgagactccaaagatagtcaagggtttgggaagagtaaatttaagccaattcagtaaattaggtgataaacaaagaagcttgctgtttttctctctttttttatgttttaaaacaatgagtttctccttttttgctcaagccctgaacaatttagttcagaatttgttttcttactttgattctgctggctttgccaattccatgaaaatccacttggcaccacatttccttagaagctcttgctttttcttgattttctttctgttcttagagtctaatattctagtgtctcaccaaattgttgttgtctctttccaaaactcctttaaccaaactatctgtatcttcatgtaaaaggcaccacagacctcccatcactcactctcccttaaccccaaaatatctatgtgttatcccttacagatgtcacttcctgcaaggccttccatattagttgctacaacatcactaaagaattttattacctgtggctctggacctacttctaatatctctggagatatccaccttcaaacacctgccacatccagctatgagggtctagtacctgggctccagagacatgttcaggttagttctgattataagaacatttctcttttttgccagaattgttggaaagttatctctattcaacagtctgtgtaggatttcagaagattgaatatacatataaatatatatacatacacacatgtatatatacacacacacatatatgtatatgtactttgtccatgtcgtatttggttattccaattctgggaaaataaattaagaaaataattaaaagatgcactcaaatatcgatatacaaaatgcctattgtaataatacataaaaaaagtcagaagctaacctcggtatctgtatgatttggagcaagttacttaaactctttgtggctatattttctcaaatgtaaaataaggttaataatagtgcttattttaaaggtagtcatgagttttaaatgaattcatgtattcaaagtggatagaatagtgctttcaaataatatttattacctaaaattattattatcatcatataagtaacaagagatacaatgaaccataatatgcccatatgataatatatcacacagtaatcaaaatattttaataggaaattaatatcataagataaaaagcaagaagaatgatgataagccatttgactaccactatatttatctatgtaactataaccacatacacatattttaaaaaattgattggaatatacaaaatggtcttttcattctctgtgatatgtaaaatgtagaaatatacttttctgggctggatgcagtagctcacacctataatcccagcactttgggaggccgaggttggtggagcacctgaggtcaggaattcgagaccagcctggccaacatagtgaaatcctgtgtctactaaaaatacaaaaattagccagatttggtggtgggtgcctgtaatcccagctactcaggaggctgaggcaggaaaattgcttgaacccaggaggcagaggttgcagtgagccgagatcctgccactgcactccagcctgggcgacagagtgagactccatctcaggaaaaaaaaaaaaaaatctatctatctatctatctatctatctatctatctatctatctatctatctatacttatatatacttttctttgttatccatattcatttcagagaatgcagtgctattgtaaaaaaaatgaataaaattaatataagtttagagcacttttttggcatcagtatgcctaaggaaaactaatgtataatacaggattaatgtttttcacaccatctcaaatcttactgaatgctgctatctagaaaacagtggtaactgagttacaatgggaccttaaaagcaaaaaaccactgtgactggctaactgaaagtgatcatagagccaaaaccataatgattgtcctgcccaagcttcagcatatatggttgcaaaatgtgcgcagtgttaatgccgtgtcaagtgtcttgtggtatttcatcagtgtcgcttacaaggcatactcaacatttaacagcaggatcattaaccatccatgctgtgatgtaatggaatagtgcttccctactaaccctgaaacaagctcactgtgtgtgacctacatccttccgctggatgagtgtgagaaaatgatgacaccacagaagcaaagcagctgctgtaagttgagccacaaggaagaaactctcaagacaggaagaataaatgctttagaggtgcactaaaataatgaatgaactaatgtggcatagctgtgaaatcatgcgaggaaaagaactacagggacagtaacagaaggtggttctttgaagtaaagacattgagaagaagaaaaattgccaacttgtgtaatcagttgatgttgctagttagccagtttaaacatgcgttaaagataaagatctgcttgaaaccaattattttattgtttggcttcctgtttaaagatgaaaatgaaatatgtgtatatatgtgacatattttacctagcagtgagtagatgcaggaagcatgtaaaatgctttgctgaattcactaacgtcacaaaatattccttaataattgccgcgtttgataggctgaccttatgctagcacaggaaacaatgcagtggacttgggttggagagaagaattttgacatttggatttttaaaaatcatgtatattgaacccatgaactctcatgtggaacatcagatttactatattttaaacagaaatgccatcactataaaaaccattgcaaatttgtcaatatacttccatttaaaaagccttttcctgcttttattgatttattgattaatttttttttgagacaggatctcactctgtcacccacgatgaagtgcagtggcacaatcgtggcttgcagcctcgacctcctaggctcaggtgatactcttacctcagctacctcccaagtaactgggaatgtaggcttgcaccaccatgtctgacccattttcttgcagaaattgagttttgctatgttgtccagacttgtcttgaactcccagactgaagcaatccgcccacttaggcctcctaaaatgttgggattacagtcatgaaccactgtgctccacctcctttttctccttttcagatagattatgtattctgtaccttttgtgtaactaacttgttattacataaactatattctaaatacattataaattattgtgatcatgatatttatcattgaattaacactgcctaagtagcaattttcacagaaaagataattttaaaagaccaaaatggttattctcttggaagcagttgacataaaataacagaataatacagattaaaattctgttttccaacagctactagtggtaagaaatatagtaagcttctgatttgtagaaaccttagctttctcatctgatgaggtggttaataatattaactctcatatttgctgaaataatataaaatatttaatataaattgtcaattataatgactggcataaggtagaattacaacaaatgctaattatatttgcttgtttgaaaatttaacactctattaaaaggcaaaaaagtgctcacctgcaaatattaatacatgccttctgtttagtccatagaaagcccacaagtaagagattcaaaagtgaaagctatcacttacagaagattaagaatatattgcattgagaagatagggattaaggagtttatcaaaggttttttaaaaaattattttcctgaacatcatttcaagaaagataagcgccaaagacataaatatattctccagagcatgtgcactgcaagttgacaaatgtgtctcttttatagagtttggtgaaggttattcattttatttttcctgtcttgaccagaaaattgtggcttctcatcatgtatgactgcctgcagggtcttctgaataggaaattaccaatgctgaaatttgtatgacttaaataagtgttgaagtgtgggggttgaaaggagatacatatcagagacatccattcagttaatagaactaagtttttgatttgcatagcttatatctagagcattgtgtttcttattttctatattactatcttctctaactatataattagcacagattgaagctataatggatttcaataatagcactactttagaatgatgtatacgtctttaaatcttaagacaagcataggaaatatgtatattcttaaaaaaaggaaaatttttttaaagtttagagttaaaagaaacatgcattttctgttacatttcagattattttagttaaagaatcttctatgctttttttcttcattcttagaagactacctgcaattgtaaagcatgaatcctgtaatacttctgtaaatgaatcactattgcatttataccattagcctctgaacagatttcaagttgctgctgccaactctcgcgagctttgtcagtaacagttgattgttacattcagtaacactgaatg